(19) United States
(12) Patent Application Publication
(10) Pub. No.: US 2015/0065436 A1
(43) Pub. Date: Mar. 5, 2015

(54) INHIBITING INTERACTION BETWEEN HIF-1ALPHA AND p300/CBP WITH HYDROGEN BOND SURROGATE-BASED HELICES

(71) Applicants: Paramjit S. ARORA, Cold Spring Harbor, NY (US); Brooke Bullock LAO, Leesville, SC (US); Laura K. HENCHEY, New York, NY (US); Nathaniel J. TRAASETH, New York, NY (US); Paul THOMSON, New York, NY (US); Swati KUSHAL, Los Angeles, CA (US); Bogdan Z. OLENYUK, Sierra Madre, CA (US)

(72) Inventors: Paramjit S. ARORA, Cold Spring Harbor, NY (US); Brooke Bullock LAO, Leesville, SC (US); Laura K. HENCHEY, New York, NY (US); Nathaniel J. TRAASETH, New York, NY (US); Paul THOMSON, New York, NY (US); Swati KUSHAL, Los Angeles, CA (US); Bogdan Z. OLENYUK, Sierra Madre, CA (US)

(73) Assignees: UNIVERSITY OF SOUTHERN CALIFORNIA, Los Angeles, CA (US); NEW YORK UNIVERSITY, New York, NY (US)

(21) Appl. No.: 14/476,394

(22) Filed: Sep. 3, 2014

Related U.S. Application Data
(60) Provisional application No. 61/873,322, filed on Sep. 3, 2013.

(51) Int. Cl. C07K 7/06; G01N 33/68

(52) U.S. Cl. CPC C07K 7/06; G01N 33/6872
USPC 514/21.6; 530/328; 435/375; 435/367; 435/369; 436/501

(57) ABSTRACT

The present invention relates to peptidomimetics that mimic helix αB of the C-terminal transactivation domain of HIF-1α. Methods of using the peptidomimetics to, e.g., inhibit the HIF-1α-p300/CBP interaction, are also disclosed.

This application claims the benefit of U.S. Provisional Patent Application Ser. No. 61/873,322, filed Sep. 3, 2013, which is hereby incorporated by reference in its entirety.

This invention was made with U.S. Government support under Grant No. CHE-1161644 awarded by the U.S. National Science Foundation and Grant No. R01GM073943 awarded by the National Institutes of Health. The U.S. Government has certain rights in this invention.

### FIELD OF THE INVENTION

This invention is directed generally to methods of inhibiting the interaction between HIF-1 and p300/CBP using artificially constrained peptides and peptidomimetics that substantially mimic helix αB of the C-terminal transactivation domain of HIF-1α.

### BACKGROUND OF THE INVENTION

### The Role of HIF-1α-Coactivator Interactions in Regulation of VEGF Transcription

The interaction between the cysteine-histidine rich 1 domain (“CH1”) of the coactivator protein p300 (or the homologous CREB binding protein, CBP) and the C-terminal transactivation domain (“C-TAD,” aa 786-826 of NCBI accession number NP 001521) of the hypoxia-inducible factor 1α (“HIF-1α”) (Freedman et al., “Structural Basis for Recruitment of CBP/p300 by Hypoxia-Inducible Factor-1α,” Proc. Nat'l Acad. Sci. USA 99:5367-72 (2002); Dames et al., “Structural Basis for Hif-1α/CBP Recognition in the Cellular Hypoxic Response,” Proc. Nat'l Acad. Sci. USA 99:5271-6 (2002)) mediates transactivation of hypoxia-Inducible genes (Hirota & Semenza, “Regulation of Angiogenesis by Hypoxia-Inducible Factor 1,” Crit. Rev. Oncol. Hematol. 59:15-26 (2006); Semenza, “Targeting HIF-1 for Cancer Therapy,” Nat. Rev. Cancer 3:721-32 (2003)). Hypoxia-inducible genes are important contributors in angiogenesis and cancer metastasis, as shown in FIGS. 1A-C (Orourke et al., “Identification of Hypoxically Inducible mRNAs in HeLa Cells Using Differential-Display PCR,” Eu. J. Biochem. 241:403-10 (1996); Ivan et al., “HIFα Targeted for VHL-Mediated Destruction by Proline Hydroxylation: Implications for O₂ Sensing,” Science 292:464-8 (2001)). Under normoxia, the α-subunit of HIF-1 is successively hydroxylated at proline residues 402 and 564 by proline hydroxylases (Ivan et al., “HIFα Targeted for VHL-Mediated Destruction by Proline Hydroxylation: Implications for O₂ Sensing,” Science 292:464-8 (2001)), ubiquitinated, and then degraded by the ubiquitin-proteosome system, as shown in FIG. 2. This process, mediated by the von Hippel-Lindau tumor suppressor protein (Kaelin, “Molecular Basis of the VHL Hereditary Cancer Syndrome,” Nat. Rev. Cancer 2:673-82 (2002)), is responsible for controlling levels of HIF-1α and, as a result, the transcriptional response to hypoxia (Maxwell et al., “The Tumour Suppressor Protein VHL Targets Hypoxia-Inducible Factors for Oxygen-Dependent Proteolysis,” Nature 399:271-5 (1999)). Under hypoxic conditions, HIF-1α is no longer targeted for destruction and accumulates. Heterodimerization with its constitutively expressed binding partner, aryl hydrocarbon receptor nuclear translocator (“ARNT”) (Wood et al., “The Role of the Aryl Hydrocarbon Receptor Nuclear Translocator (ARNT) in Hypoxic Induction of Gene Expression,” J. Biol. Chem. 271:15117-23 (1996)) results in binding to a cognate hypoxia response element (“HRE”) (Forsythe et al., “Activation of Vascular Endothelial Growth Factor Gene Transcription by Hypoxia-Inducible Factor 1,” Mol. Cell. Biol. 16:4604-13 (1996)). A third site of regulatory hydroxylation on asparagine 803 is also inhibited under hypoxic conditions (Lando et al., “FIH-1 Is an Asparaginyl Hydroxylase Enzyme That Regulates the Transcriptional Activity of Hypoxia-Inducible Factor,” Genes & Develop. 16:1466-71 (2002)), allowing recruitment of the p300/CBP coactivators, which trigger overexpression of hypoxia inducible genes, as shown in FIG. 2. Among these are genes encoding angiogenic peptides such as vascular endothelial growth factor (“VEGF”) and VEGF receptors VEGFR-1 (Flt-1) and VEGFR-2 (KDR/Flk-1), as well as proteins involved in altered energy metabolism, such as the glucose transporters GLUT1 and GLUT3, and hexokinases 1 and 2 (Forsythe et al., “Activation of Vascular Endothelial Growth Factor Gene Transcription by Hypoxia-Inducible Factor 1,” Mol. Cell. Biol. 16:4604-13 (1996); Okino et al., “Hypoxia-Inducible Mammalian Gene Expression Analyzed in Vivo at a TATA-Driven Promoter and at an Initiator-Driven Promoter,” J. Biol. Chem. 273:23837-43 (1998)).

### Epidithiodiketopiperazine Fungal Metabolites as Regulators of Hypoxia-Inducible Transcription

Because interaction of HIF-1α C-TAD with transcriptional coactivator p300/CBP is a point of significant amplification in transcriptional response, its disruption with designed protein ligands can be an effective means of suppressing aerobic glycolysis and angiogenesis (i.e., the formation of new blood vessels) in cancers (Hirota & Semenza, “Regulation of Angiogenesis by Hypoxia-Inducible Factor 1,” Crit. Rev. Oncol. Hematol. 59:15-26 (2006); Rarnanathan et al., “Perturbational Profiling of a Cell-Line Model of Tumorigenesis by Using Metabolic Measurements,” Proc. Nat'l Acad. Sci. USA 102:5992-7 (2005); Underiner et al., “Development of Vascular Endothelial Growth Factor Receptor (VEGFR) Kinase Inhibitors as Anti-Angiogenic Agents in Cancer Therapy,” Curr. Med. Chem. 11:731-45 (2004)). Although the contact surface of the HIF-1α C-TAD with p300/CBP is extensive (3393 Å²), the inhibition of this protein-protein interaction may not require direct interference. Instead, the induction of a structural change to one of the binding partners (p300/CBP) may be sufficient to disrupt the complex (Kung et al., “Small Molecule Blockade of Transcriptional Coactivation of the Hypoxia-Inducible Factor Pathway,” Cancer Cell 6:33-43 (2004)).

The present invention is directed to overcoming these and other deficiencies in the art.

### SUMMARY OF THE INVENTION

A first aspect of the present invention relates to a peptidomimetic, wherein the peptidomimetic:

(i) mimics a helix having the formula X₁—X₂—X₂—X₃—X₂—X₂—X₁—X₄—X₅, wherein each X₁ is any negatively charged residue, each X₂ is any hydrophobic residue, X₃ is any positively-charged residue, X₄ is any polar residue, and X₅ is absent or any hydrophobic residue; and
(ii) is selected from the group consisting of:

(a) a compound of Formula I:

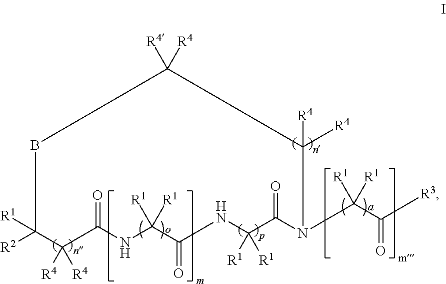

wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

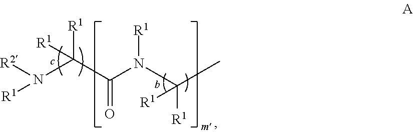

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

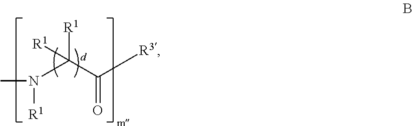

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
a is one or two;
m, n′, and n″ are each independently zero, one, two, three, or four;
m′″ is zero or one;
each o is independently one or two; and
p is one or two;

(b) a compound of Formula II:

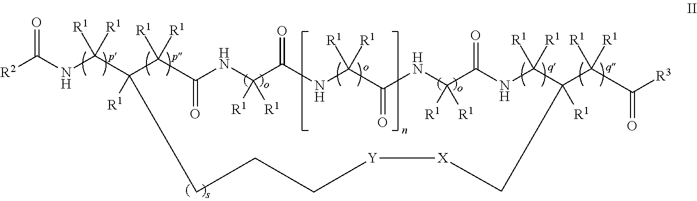

wherein:
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

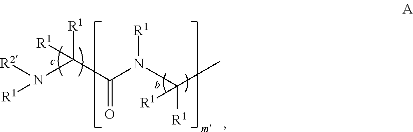

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

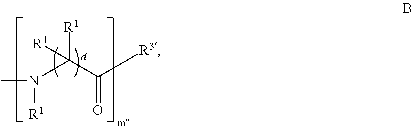

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
n is one or four;
each o is independently one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond; and

(c) a compound of Formula III:

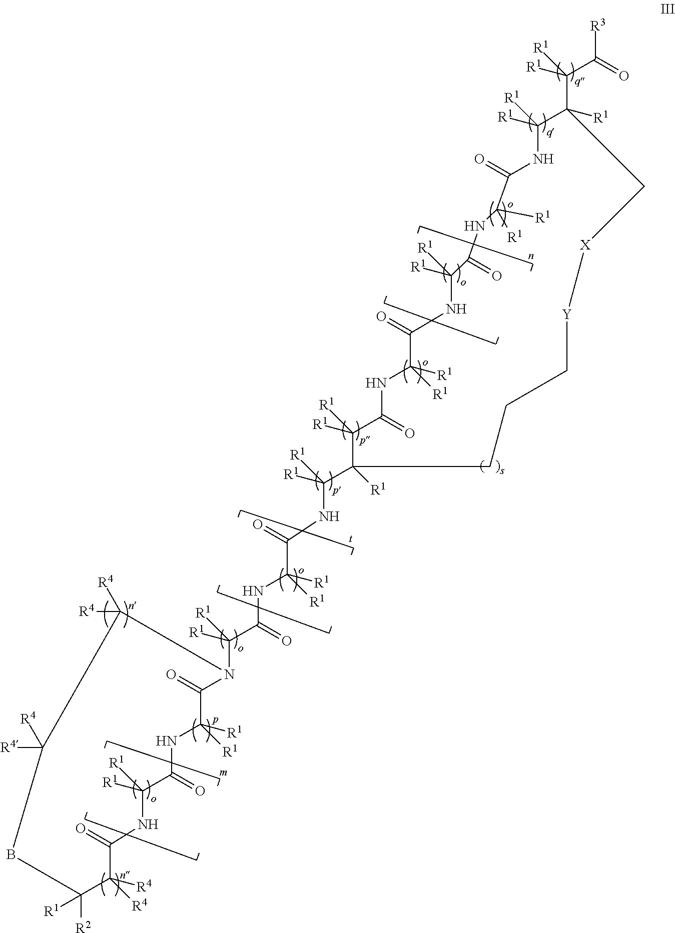

wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

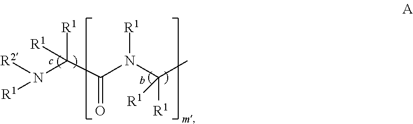

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

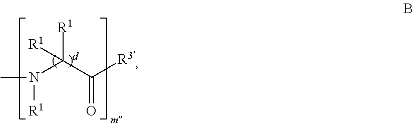

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
m, n′, and n″ are each independently zero, one, two, three, or four;
n is one or four;
each o is independently one or two;
p is one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond.

A second aspect of the present invention relates to a method of modulating transcription of a gene in a cell, where transcription of the gene is mediated by interaction of hypoxia-inducible factor 1α (“HIF-1α”) with coactivator protein p300 (or the homologous CREB binding protein, CBP). This method involves contacting the cell with a peptidomimetic described herein under conditions effective to modulate transcription of the gene.

A third aspect of the present invention relates to a method of treating or preventing in a subject a disorder mediated by interaction of HIF-1α with CBP and/or p300. This method involves administering a peptidomimetic described herein to the subject under conditions effective to treat or prevent the disorder.

A fourth aspect of the present invention relates to a method of reducing or preventing angiogenesis in a tissue. This method involves contacting the tissue with a peptidomimetic described herein under conditions effective to reduce or prevent angiogenesis in the tissue.

A fifth aspect of the present invention relates to a method of decreasing survival and/or proliferation of a cell under hypoxic conditions. This method involves contacting the cell with a peptidomimetic described herein under conditions effective to decrease survival and/or proliferation of the cell.

A sixth aspect of the present invention relates to a method of identifying a potential ligand of CBP and/or p300. This method involves providing a peptidomimetic described herein, contacting the peptidomimetic with a test agent, and detecting whether the test agent selectively binds to the peptidomimetic. A test agent that selectively binds to the peptidomimetic is identified as a potential ligand of CBP and/or p300.

Selective blockade of gene expression by designed small molecules is a fundamental challenge at the interface of chemistry, biology, and medicine. Transcription factors have been among the most elusive targets in genetics and drug discovery, but the fields of chemical biology and genetics have evolved to a point where this task can be addressed. The design, synthesis, and in vivo efficacy evaluation of a protein domain mimetic targeting the interaction of the p300/CBP coactivator with the transcription factor HIF-1α is described herein. As indicated herein, disrupting this interaction results in a rapid down-regulation of hypoxia-inducible genes critical for cancer progression. The observed effects were compound-specific and dose-dependent. Gene expression profiling with oligonucleotide microarrays revealed effective inhibition of hypoxia-inducible genes with relatively minimal perturbation of non-targeted signaling pathways. Remarkable efficacy of the compound HBS 1 in suppressing tumor growth was observed in the fully established murine xenograft models of renal cell carcinoma of the clear cell type (RCC). These results suggest that rationally designed synthetic mimics of protein subdomains that target the transcription factor-coactivator interfaces represent a novel approach for in vivo modulation of oncogenic signaling and arresting tumor growth.

### BRIEF DESCRIPTION OF THE DRAWINGS

FIG. 1A is a schematic diagram illustrating the structure of the complex of the C-terminal transactivation domain (“C-TAD”) of the hypoxia-inducible factor 1α (“HIF-1α”) with cysteine-histidine rich 1 domain (“CH1”) of the coactivator protein p300 (or the homologous CREB binding protein, CBP) (Lepourcelet et al., “Small-Molecule Antagonists of the Oncogenic Tcf/β-Catenin Protein Complex,” Cancer Cell 5:91-102 (2004); Vassilev et al., “In Vivo Activation of the p53 Pathway by Small-Molecule Antagonists of MDM2,” Science 303:844-48 (2004), which are hereby incorporated by reference in their entirety). FIG. 1B is the domain map of HIF-1α showing the basic helix-loop-helix region (“bHLH”), PAS, the N-terminal transactivation domain (“N-TAD”), and the C-TAD. The human HIF-1α C-TAD sequence (SEQ ID NO: 1) is shown in FIG. 1C, along with the location of the αA and αB helices.

FIG. 2 is a schematic diagram illustrating the HIF-1α pathway. ARNT: aryl hydrocarbon receptor nuclear translocator; VHL: von Hippel-Lindau tumor suppressor; HRE: hypoxia response element; VEGF: vascular endothelial growth factor.

FIGS. 3A-C are schematic diagrams relating to the regulation of transcription by HIF-1α and CBP/p300. As shown in FIG. 3A, transcription of hypoxia-inducible genes is controlled by the interaction of HRE-bound HIF-1α/ARNT heterodimer with transcriptional coactivator CBP/p300. Protein domain mimetics should competitively inhibit the interaction and associated gene expression (see FIG. 3B). As shown in FIG. 3C, the C-TAD₇₉₃₋₈₂₆ domain of HIF-1α(SEQ ID NO: 2) utilizes helical motifs to target the cysteine-histidine rich 1 (CH1) region of CBP/p300. HIF-1α is shown in gold and CBP/p300 in gray (PDB code 1L8C).

FIGS. 4A-C are analytical HPLC traces of HBS 1 (FIG. 4A), HBS 2 (FIG. 4B), and peptide 3 (FIG. 4C).

FIGS. 5A-C show that HBS 1 targets p300-CH1 with high affinity and inhibits its binding to HIF-1α C-TAD₇₈₆₋₈₂₆. FIG. 5A is a graph of the affinity of HBS 1, HBS 2, peptide 3, and HIF-1αC-TAD₇₈₆₋₈₂₆ for the CH1 domain as determined by tryptophan fluorescence spectroscopy. FIG. 5B is a molecular model that depicts the results of a ¹H-¹⁵N HSQC NMR titration experiment. The p300-CH1 residues undergoing chemical shift perturbations upon addition of HBS 1 are color-mapped, matching the magnitude of the chemical shift changes. HIF-1α helix B is shown in gold. The model was refined from the NMR structure of the HIF-1α/p300 complex (PDB code 1L8C). FIG. 5C is a graph of the results of fluorescence anisotropy experiments, showing the ability of HBS 1 to inhibit CH1^{Flu}/HIF C-TAD₇₈₆₋₈₂₆ complex formation.

FIGS. 6A-B show the structures of stabilized helices and linear peptide. HBS 1 (FIG. 6A, left panel) mimics the αB domain of HIF-1α and features four residues that contribute significantly to binding (L818, L822, L823 and L824). HBS 2 (FIG. 6A, right panel) was designed to be a specificity control; this compound is identical to HBS 1 with the exception of L822, which was mutated to an alanine group. Peptide 3 (FIG. 6B) (SEQ ID NO: 3) is an unconstrained negative control with the amino acid sequence that repeats that of HBS 1.

FIG. 7 is the circular dichroism spectra of HBS 1, HBS 2, and peptide 3. CD studies were performed with 50-100 μM peptide solutions in 10 mM KF (pH 7.4).

FIGS. 8A-D are ¹H-¹⁵N HSQC spectra of the p300-CH1 domain with different concentrations of Zn²⁺. FIG. 8A is the spectra of misfolded p300-CH1:Zn²⁺ (1:<3). FIG. 8B is the spectra of folded p300-CH1:Zn²⁺ (1:3). FIG. 8C is the spectra of unfolded p300-CH1:Zn²⁺ with excess Zn²⁺ (1:6). FIG. 8D is the spectra of refolded p300-CH1:Zn²⁺ with EDTA to remove the excess of Zn²⁺ (1:3).

FIG. 9 is a schematic diagram of the HIF-1α/p300-CH1 interaction. Tryptophan-403 resides in the hydrophobic groove targeted by the HIF-1α αB helix. (PDB code 1L8C.)

FIG. 10 is a graph showing the concentration-dependent changes in the fluorescence spectra of the CH1 domain (1 μM) upon titration of HBS 1.

FIG. 11 shows the chemical structure of fluorescein-labeled C-TAD (Flu-HIF-1α C-TAD₇₈₆₋₈₂₆). (Mass [M+H] calc'd=4977.1. found=4976.8.)

FIG. 12 is a graph of the binding of Flu-HIF C-TAD to p300-CH1 as monitored by a fluorescence polarization assay.

FIG. 13 is the overlaid ¹H-¹⁵N HSQC titration spectra of p300-CH1 (blue), CH1:HBS 1 (1:5, red), and CH1:HBS 1 (1:10, green).

FIG. 14 is a mean chemical shift difference (ΔδNTH) plot depicting changes in residues of p300-CH1 upon binding with HBS 1.

FIG. 15 is a graph of the results from the luciferase-based promoter activity assay with MDA-MB-231-HRE-Luc cell line treated with HBS 1, HBS 2 (specificity control), or peptide 3. Hypoxia was mimicked with GasPak EZ pouch (300 μM). Error bars represent ±s.e.m. of experiments performed in quadruplicate. * P<0.05, t-test. The results demonstrate that HBS 1 reduces HIF-1α inducible promoter activity in vitro.

FIG. 16 is a western blot analysis of HIF-1α levels in the nuclear and cytoplasmic extracts of HeLa cells. Cells were incubated for a total of 24 hours with HBS 1. After 6 hours, hypoxia was mimicked with DFO (300 μM) for an additional 18 hours. The results demonstrate that HBS 1 does not affect the intracellular levels of HIF-1α.

FIGS. 17A-D show that HBS 1 down-regulates hypoxia-induced transcription in cell culture. As shown in FIGS. 17A-C, HBS 1 reduced expression levels of VEGFA (FIG. 17A), SLC2A1 (GLUT1) (FIG. 17B), and LOX (FIG. 17C) in a dose-dependent manner in HeLa cells under hypoxia conditions as measured by real-time qRT-PCR. Hypoxia was mimicked with DFO (300 μM). HBS 2 and peptide 3 show reduced inhibitory activities at the same concentrations. Error bars are ±s.e.m. of four independent experiments. ** P<0.01, * P<0.05, t-test. FIG. 17D is a graph comparing the efficacies of HBS 1 in down-regulating expression levels of VEGFA in HeLa cells under two different hypoxia-mimetic conditions (DFO and hypoxia bag) as measured by real-time qRT-PCR. For each experiment under hypoxia-mimetic conditions, mRNA levels were normalized to VEGFA mRNA levels found in the vehicle-treated normoxic cells.

FIG. 18 is a graph of VEGF protein levels under hypoxia or normoxia, with or without treatment with varying concentrations of HBS 1. Hypoxia was mimicked with 300 μM DFO. Error bars represent ±s.e.m of experiments performed in triplicate. * P<0.05, t-test. The results demonstrate that HBS 1 reduces levels of secreted VEGF protein in HeLa cells in a dose-dependent manner.

FIG. 19 is a graph of the results from MTT assays with HeLa cells treated with HBS 1, HBS 2, or peptide 1 in a concentration range of 1 μM and 100 μM for 24 hours. The results demonstrate that HBS 1 shows low cytotoxicity in HeLa cells.

FIGS. 20A-C show the results from gene expression profiling obtained with Affymetrix Human Gene ST 1.0 arrays. FIG. 20A shows the hierarchical agglomerative clustering of 368 transcripts induced or repressed 2-fold or more (one-way ANOVA, P<0.05) by 300 μM DFO under the three specified conditions: no treatment (“-”), treatment with 50 μM HBS 1 (“1”), and treatment with 50 μM HBS 2 (“2”). Clustering was based on a Pearson centered correlation of intensity ratios for each treatment compared to DFO-induced cells (controls) using average-linkage as a distance. Of this DFO-induced set, 92 were inhibited and 30 were induced by HBS 1, whereas 81 were inhibited and 70 induced by HBS 2 (|fold-change|≧1.1, P<0.05). FIG. 20B shows a clustering of expression changes of the 45 transcripts induced or repressed 4-fold or more (P<0.05) by 300 μM DFO or by the treatments under the designated treatment conditions. Clustering parameters were the same as in FIG. 20A. FIG. 20C shows Venn diagrams representing transcripts down- and up-regulated (|fold-change|≧1.1, P<0.05) by HBS 1 and HBS 2. Numbers inside the intersections represent DFO-induced transcripts affected by both treatments.

FIG. 21 shows the plasma concentration versus time curves for HBS 1 and control peptide 3 in BALB/c mice.

FIGS. 22A-C demonstrate that HBS 1 suppresses tumor growth in mouse xenograft models. FIG. 22A is a box-whisker diagram of tumor volumes measured throughout the study with boxes representing the upper and lower quartiles and median and error bars showing maximum and minimum volumes. Tumors from mice treated with HBS 1 were smaller (median volume: 138 mm³) than those of the control mice (median: 293 mm³). FIG. 22B is a graph showing the results of the weight measurements of control- and HBS 1-treated mice throughout the entire duration of the experiments, showing the absence of toxicity-related weight loss. FIG. 22C shows images of mice injected with the tumor-accumulating near-infrared (NIR) contrast agent. Mice from the HBS 1 treated group show significantly lower intensity of the NIR signal as compared to the control group, demonstrating that HBS 1 lowers overall tumor burden in mice.

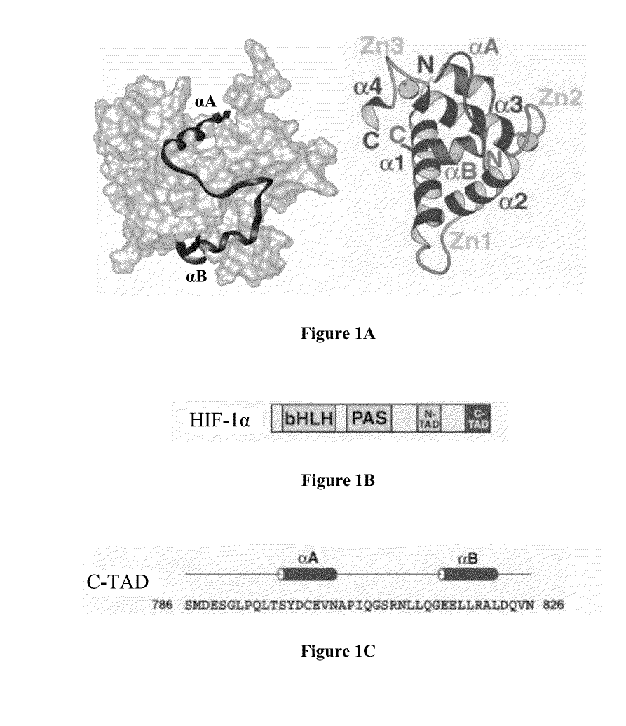

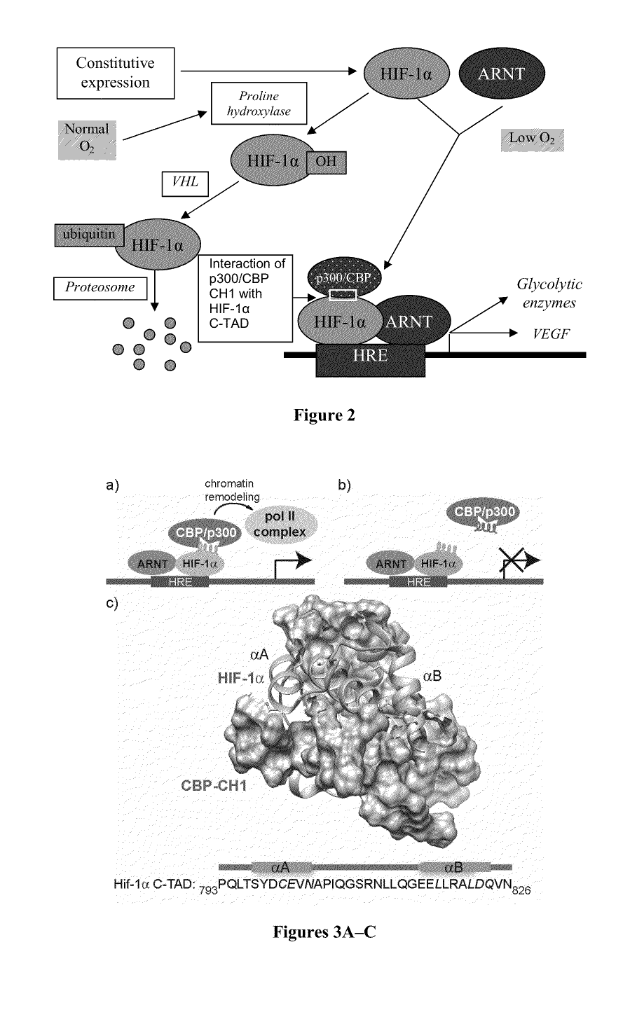

### DETAILED DESCRIPTION OF THE INVENTION

Transcription factors are among the most challenging, but attractive targets, for drug discovery (Rutledge et al., “Molecular Recognition of Protein Surfaces: High Affinity Ligands for the CBPKIX Domain,” J. Am. Chem. Soc. 125(47):14336-47 (2003), which is hereby incorporated by reference in its entirety). High-resolution structures of transcription factors in complex with protein partners offer a foundation for rational drug design strategies. Although many transcription factors exhibit significant intrinsic disorder, their complexes with coactivator proteins often feature discrete protein secondary structures (Rutledge et al., “Molecular Recognition of Protein Surfaces: High Affinity Ligands for the CBPKIX Domain,” J. Am. Chem. Soc. 125(47):14336-47 (2003), which is hereby incorporated by reference in its entirety), such as α-helices, that contribute significantly to binding and may be used as templates for rational drug design (Semenza, “Targeting HIF-1 for Cancer Therapy,” Nat. Rev. Cancer 3(10):721-32 (2003), which is hereby incorporated by reference in its entirety). Described herein is the design of stabilized peptide α-helices that can modulate transcription of hypoxia inducible genes by interfering with interactions of the C-terminal activation domain (“C-TAD”) of hypoxia inducible factor-1α (“HIF-1α”) and the cysteine-histidine rich 1 (“CH1”) domain of the coactivator protein p300 (or the homologous CREB binding protein, CBP) (FIGS. 3A-C) (O'Rourke et al., “Identification of Hypoxically Inducible mRNAs in HeLa Cells Using Differential-Display PCR: Role of Hypoxia-Inducible Factor-1,” Eur. J. Biochem. 241(2):403-10 (1996); Freedman et al., “Structural Basis for Recruitment of CBP/p300 by Hypoxia-Inducible Factor-1 Alpha,” Proc. Nat'l Acad. Sci. U.S.A. 99(8):5367-72 (2002), which are hereby incorporated by reference in their entirety). As shown herein, an optimized mimic of HIF-1αC-TAD, HBS 1, a high affinity ligand of CH1, can downregulate target genes under hypoxic conditions without affecting the endogenous levels of HIF-1α. HBS 1 does not adversely affect cell growth at high concentrations, which suggests that the compound is generally non-toxic to normoxic cells. This constrained α-helix retains significant activity in mouse plasma as compared to its unconstrained peptide analog (peptide 3) highlighting the ability of stabilized helices to evade serum proteases. The genome-wide effects of HIF-1αC-TAD mimic 1 and a negative control (HBS 2) were compared using gene expression profiling. The results show that HBS 1 modulates expression of a select set of genes, many of which are of direct relevance to the predicted pathways. Lastly, the ability of HBS 1 to control tumor progression in a mouse tumor xenograft model was examined. The synthetic helix was found to provide rapid and effective regression of tumor growth. These results support the hypothesis that functional mimics of protein subdomains that mediate interactions between partner proteins offer an attractive strategy for inhibitor design. It is predicted that other such peptidomimetics of the αB helix of HIF-1α would have similar effects.

The present invention relates to a peptidomimetic, wherein the peptidomimetic:

(i) mimics a helix having the formula X₁—X₂—X₂—X₃—X₂—X₂—X₁—X₄—X₅, wherein each X₁ is any negatively charged residue, each X₂ is any hydrophobic residue, X₃ is any positively-charged residue, X₄ is any polar residue, and X₅ is absent or any hydrophobic residue; and
(ii) is selected from the group consisting of:

(a) a compound of Formula I:

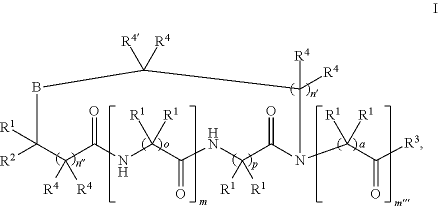

wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

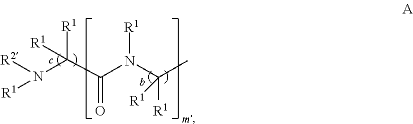

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

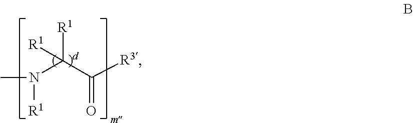

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
a is one or two;
m, n′, and n″ are each independently zero, one, two, three, or four;
m′″ is zero or one;
each o is independently one or two; and
p is one or two;

(b) a compound of Formula II:

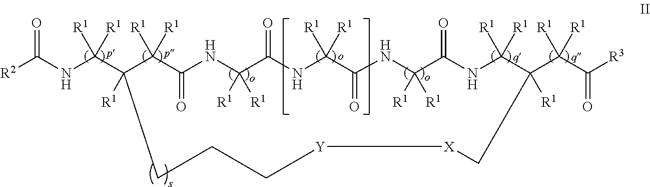

wherein:
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

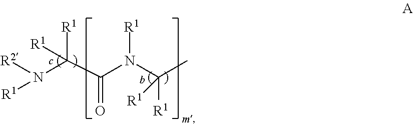

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

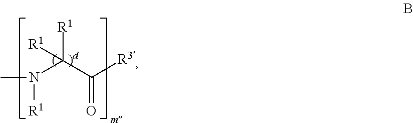

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
n is one or four;
each o is independently one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond; and

(c) a compound of Formula III:

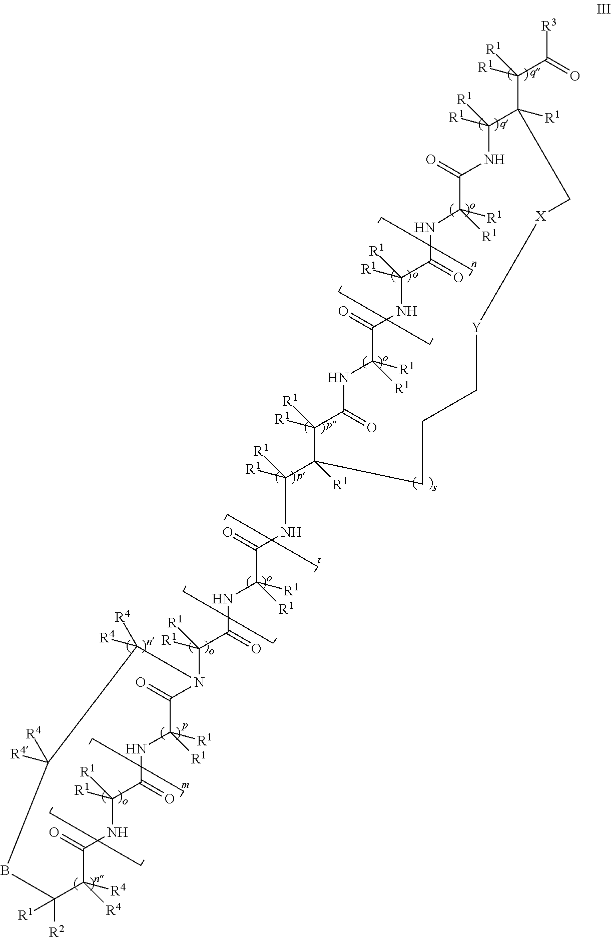

wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A:

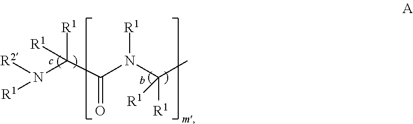

wherein:
   R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m′ is zero or any number;
   each b is independently one or two; and
   c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B:

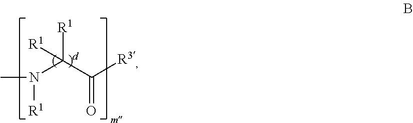

wherein:
   R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
   m″ is zero or any number; and
   each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
m, n′, and n″ are each independently zero, one, two, three, or four;
n is one or four;
each o is independently one or two;
p is one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond.

Amino acid side chains according to this and all aspects of the present invention can be any amino acid side chain from natural or nonnatural amino acids, including from alpha amino acids, beta amino acids, gamma amino acids, L-amino acids, and D-amino acids.

As used herein, the term “alkyl” means an aliphatic hydrocarbon group which may be straight or branched having about 1 to about 6 carbon atoms in the chain. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 6 carbon atoms in the chain. Preferred alkenyl groups have 2 to about 4 carbon atoms in the chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having about 2 to about 6 carbon atoms in the chain. Preferred alkynyl groups have 2 to about 4 carbon atoms in the chain. Exemplary alkynyl groups include ethynyl, propynyl, n-butynyl, 2-butynyl, 3-methylbutynyl, and n-pentynyl.

As used herein, the term “cycloalkyl” refers to a non-aromatic saturated or unsaturated mono- or polycyclic ring system which may contain 3 to 6 carbon atoms, and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

As used herein, the term “heterocyclyl” refers to a stable 3- to 18-membered ring system that consists of carbon atoms and from one to five heteroatoms selected from the group consisting of nitrogen, oxygen, and sulfur. The heterocyclyl may be a monocyclic or a polycyclic ring system, which may include fused, bridged, or spiro ring systems; and the nitrogen, carbon, or sulfur atoms in the heterocyclyl may be optionally oxidized; the nitrogen atom may be optionally quaternized; and the ring may be partially or fully saturated. Representative monocyclic heterocyclyls include piperidine, piperazine, pyrimidine, morpholine, thiomorpholine, pyrrolidine, tetrahydrofuran, pyran, tetrahydropyran, oxetane, and the like. Representative polycyclic heterocyclyls include indole, isoindole, indolizine, quinoline, isoquinoline, purine, carbazole, dibenzofuran, chromene, xanthene, and the like.

As used herein, the term “aryl” refers to an aromatic monocyclic or polycyclic ring system containing from 6 to 19 carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein, “heteroaryl” refers to an aromatic ring radical which consists of carbon atoms and from one to five heteroatoms selected from the group consisting of nitrogen, oxygen, and sulfur. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl. Additional heteroaryls are described in COMPREHENSIVE HETEROCYCLIC CHEMISTRY: THE STRUCTURE, REACTIONS, SYNTHESIS AND USE OF HETEROCYCLIC COMPOUNDS (Katritzky et al. eds., 1984), which is hereby incorporated by reference in its entirety.

The term “arylalkyl” refers to a moiety of the formula —R^{a}R^{b} where R^{a} is an alkyl or cycloalkyl as defined above and R^{b} is an aryl or heteroaryl as defined above.

As used herein, the term “acyl” means a moiety of formula R-carbonyl, where R is an alkyl, cycloalkyl, aryl, or heteroaryl as defined above. Exemplary acyl groups include formyl, acetyl, propanoyl, benzoyl, and propenoyl.

An amino acid according to this and all aspects of the present invention can be any natural or non-natural amino acid.

A “peptide” as used herein is any oligomer of two or more natural or non-natural amino acids, including alpha amino acids, beta amino acids, gamma amino acids, L-amino acids, D-amino acids, and combinations thereof. In preferred embodiments, the peptide is ˜5 to ˜30 (e.g., ˜5 to ˜10, ˜5 to ˜17, ˜10 to ˜17, ˜10 to ˜30, or ˜18 to ˜30) amino acids in length. Typically, the peptide is 10-17 amino acids in length. In a preferred embodiment, the peptide contains a mixture of alpha and beta amino acids in the pattern α3/β1 (this is particularly preferred for α-helix mimetics).

A “tag” as used herein includes any labeling moiety that facilitates the detection, quantitation, separation, and/or purification of the compounds of the present invention. Suitable tags include purification tags, radioactive or fluorescent labels, and enzymatic tags.

Purification tags, such as poly-histidine (His₆₋), a glutathione-S-transferase (GST-), or maltose-binding protein (MBP-), can assist in compound purification or separation but can later be removed, i.e., cleaved from the compound following recovery. Protease-specific cleavage sites can be used to facilitate the removal of the purification tag. The desired product can be purified further to remove the cleaved purification tags.

Other suitable tags include radioactive labels, such as, ¹²⁵I, ¹³¹I, ¹¹¹In, or ⁹⁹TC. Methods of radiolabeling compounds are known in the art and described in U.S. Pat. No. 5,830,431 to Srinivasan et al., which is hereby incorporated by reference in its entirety. Radioactivity is detected and quantified using a scintillation counter or autoradiography. Alternatively, the compound can be conjugated to a fluorescent tag. Suitable fluorescent tags include, without limitation, chelates (europium chelates), fluorescein and its derivatives, rhodamine and its derivatives, dansyl, Lissamine, phycoerythrin, and Texas Red. The fluorescent labels can be conjugated to the compounds using techniques disclosed in CURRENT PROTOCOLS IN IMMUNOLOGY (Coligen et al. eds., 1991), which is hereby incorporated by reference in its entirety. Fluorescence can be detected and quantified using a fluorometer.

Enzymatic tags generally catalyze a chemical alteration of a chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Examples of suitable enzymatic tags include luciferases (e.g., firefly luciferase and bacterial luciferase; see e.g., U.S. Pat. No. 4,737,456 to Weng et al., which is hereby incorporated by reference in its entirety), luciferin, 2,3-dihydrophthalazinediones, malate dehydrogenase, urease, peroxidases (e.g., horseradish peroxidase), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (e.g., uricase and xanthine oxidase), lactoperoxidase, microperoxidase, and the like. Techniques for conjugating enzymes to proteins and peptides are described in O'Sullivan et al., Methods for the Preparation of Enzyme—Antibody Conjugates for Use in Enzyme Immunoassay, in METHODS IN ENZYMOLOGY 147-66 (Langone et al. eds., 1981), which is hereby incorporated by reference in its entirety.

A targeting moiety according to the present invention functions to (i) promote the cellular uptake of the compound, (ii) target the compound to a particular cell or tissue type (e.g., signaling peptide sequence), or (iii) target the compound to a specific sub-cellular localization after cellular uptake (e.g., transport peptide sequence).

To promote the cellular uptake of a compound of the present invention, the targeting moiety may be a cell penetrating peptide (CPP). CPPs translocate across the plasma membrane of eukaryotic cells by a seemingly energy-independent pathway and have been used successfully for intracellular delivery of macromolecules, including antibodies, peptides, proteins, and nucleic acids, with molecular weights several times greater than their own. Several commonly used CPPs, including polyarginines, transportant, protamine, maurocalcine, and M918, are suitable targeting moieties for use in the present invention and are well known in the art (see Stewart et al., “Cell-Penetrating Peptides as Delivery Vehicles for Biology and Medicine,” Organic Biomolecular Chem. 6:2242-55 (2008), which is hereby incorporated by reference in its entirety). Additionally, methods of making CPP are described in U.S. Patent Application Publication No. 20080234183 to Hallbrink et al., which is hereby incorporated by reference in its entirety.

Another suitable targeting moiety useful for enhancing the cellular uptake of a compound is an “importation competent” signal peptide as disclosed by U.S. Pat. No. 6,043,339 to Lin et al., which is hereby incorporated by reference in its entirety. An importation competent signal peptide is generally about 10 to about 50 amino acid residues in length—typically hydrophobic residues—that render the compound capable of penetrating through the cell membrane from outside the cell to the interior of the cell. An exemplary importation competent signal peptide includes the signal peptide from Kaposi fibroblast growth factor (see U.S. Pat. No. 6,043,339 to Lin et al., which is hereby incorporated by reference in its entirety). Other suitable peptide sequences can be selected from the SIGPEP database (see von Heijne G., “SIGPEP: A Sequence Database for Secretory Signal Peptides,” Protein Seq. Data Anal. 1(1):41-42 (1987), which is hereby incorporated by reference in its entirety).

Another suitable targeting moiety is a signal peptide sequence capable of targeting the compounds of the present invention to a particular tissue or cell type. The signaling peptide can include at least a portion of a ligand binding protein. Suitable ligand binding proteins include high-affinity antibody fragments (e.g., Fab, Fab′ and F(ab)₂, single-chain Fv antibody fragments), nanobodies or nanobody fragments, fluorobodies, or aptamers. Other ligand binding proteins include biotin-binding proteins, lipid-binding proteins, periplasmic binding proteins, lectins, serum albumins, enzymes, phosphate and sulfate binding proteins, immunophilins, metallothionein, or various other receptor proteins. For cell specific targeting, the signaling peptide is preferably a ligand binding domain of a cell specific membrane receptor. Thus, when the modified compound is delivered intravenously or otherwise introduced into blood or lymph, the compound will adsorb to the targeted cell, and the targeted cell will internalize the compound. For example, if the target cell is a cancer cell, the compound may be conjugated to an anti-C3B(I) antibody as disclosed by U.S. Pat. No. 6,572,856 to Taylor et al., which is hereby incorporated by reference in its entirety. Alternatively, the compound may be conjugated to an alphafeto protein receptor as disclosed by U.S. Pat. No. 6,514,685 to Moro, which is hereby incorporated by reference in its entirety, or to a monoclonal GAH antibody as disclosed by U.S. Pat. No. 5,837,845 to Hosokawa, which is hereby incorporated by reference in its entirety. For targeting a compound to a cardiac cell, the compound may be conjugated to an antibody recognizing elastin microfibril interfacer (EMILIN2) (Van Hoof et al., “Identification of Cell Surface for Antibody-Based Selection of Human Embryonic Stem Cell-Derived Cardiomyocytes,” J Proteom Res 9:1610-18 (2010), which is hereby incorporated by reference in its entirety), cardiac troponin I, connexin-43, or any cardiac cell-surface membrane receptor that is known in the art. For targeting a compound to a hepatic cell, the signaling peptide may include a ligand domain specific to the hepatocyte-specific asialoglycoprotein receptor. Methods of preparing such chimeric proteins and peptides are described in U.S. Pat. No. 5,817,789 to Heartlein et al., which is hereby incorporated by reference in its entirety.

Another suitable targeting moiety is a transport peptide that directs intracellular compartmentalization of the compound once it is internalized by a target cell or tissue. For transport to the endoplasmic reticulum (ER), for example, the compound can be conjugated to an ER transport peptide sequence. A number of such signal peptides are known in the art, including the signal peptide MMSFVSLLLVGILFYATEAEQLTKCEVFQ (SEQ ID NO: 4). Other suitable ER signal peptides include the N-terminus endoplasmic reticulum targeting sequence of the enzyme 17β-hydroxysteroid dehydrogenase type 11 (Horiguchi et al., “Identification and Characterization of the ER/Lipid Droplet-Targeting Sequence in 17β-hydroxysteroid Dehydrogenase Type 11,” Arch. Biochem. Biophys. 479(2):121-30 (2008), which is hereby incorporated by reference in its entirety), or any of the ER signaling peptides (including the nucleic acid sequences encoding the ER signal peptides) disclosed in U.S. Patent Application Publication No. 20080250515 to Reed et al., which is hereby incorporated by reference in its entirety. Additionally, the compound of the present invention can contain an ER retention signal, such as the retention signal KEDL (SEQ ID NO: 5). Methods of modifying the compounds of the present invention to incorporate transport peptides for localization of the compounds to the ER can be carried out as described in U.S. Patent Application Publication No. 20080250515 to Reed et al., which is hereby incorporated by reference in its entirety.

For transport to the nucleus, the compounds of the present invention can include a nuclear localization transport signal. Suitable nuclear transport peptide sequences are known in the art, including the nuclear transport peptide PPKKKRKV (SEQ ID NO: 6). Other nuclear localization transport signals include, for example, the nuclear localization sequence of acidic fibroblast growth factor and the nuclear localization sequence of the transcription factor NF-KB p50 as disclosed by U.S. Pat. No. 6,043,339 to Lin et al., which is hereby incorporated by reference in its entirety. Other nuclear localization peptide sequences known in the art are also suitable for use in the compounds of the present invention.

Suitable transport peptide sequences for targeting to the mitochondria include MLSLRQSIRFFKPATRTLCSSRYLL (SEQ ID NO: 7). Other suitable transport peptide sequences suitable for selectively targeting the compounds of the present invention to the mitochondria are disclosed in U.S. Patent Application Publication No. 20070161544 to Wipf, which is hereby incorporated by reference in its entirety.

The peptidomimetics of the present invention are designed to mimic a helix having the formula X₁—X₂—X₂—X₃—X₂—X₂—X₁—X₄—X₅, wherein each X₁ is any negatively charged residue, each X₂ is any hydrophobic residue, X₃ is any positively-charged residue, X₄ is any polar residue, and X₅ is absent or any hydrophobic residue. In a preferred embodiment, the peptidomimetic mimics a helix having the formula X₁—X₂-L-X₃—X₂-L-X₁—X₄—X₅. In a preferred embodiment, the peptidomimetic mimics a helix having the formula X₁—X₂-L-X₃—X₂-L-D-X₄—X₅. In a preferred embodiment, the peptidomimetic mimics a helix having the formula X₁—X₂-L-X₃—X₂-L-X₁-Q-X₅. In a preferred embodiment, the peptidomimetic mimics a helix having the formula X₁—X₂-L-X₃—X₂-L-D-Q-X₅ (SEQ ID NO: 8). In a preferred embodiment, the peptidomimetic mimics a helix having the formula XELA*RALDQ (SEQ ID NO: 9), where X is 4-pentenoic acid and A* is N-allylalanine.

As will be apparent to those of ordinary skill in the art, when R² and/or R³ are a moiety of the recited formulae, the overall size of the compounds of Formula I, Formula II, and Formula III can be adjusted by varying the values of m′ and/or m″, which are independently zero or any number. Typically, m′ and m″ are independently from zero to about thirty (e.g., 0 to ˜18, 0 to ˜10, 0 to ˜5, ˜5 to ˜30, ˜5 to ˜18, ˜5 to ˜10, ˜8 to ˜30, ˜8 to ˜18, ˜8 to ˜10, ˜10 to ˜18, or ˜10 to ˜30). In one embodiment of compounds of Formula I, m′ and m″ are independently 4-10. In another embodiment of compounds of Formula I, m′ and m″ are independently 5-6.

As will be apparent to the skilled artisan, compounds of Formula I and Formula III include a diverse range of helical conformation, which depends on the values of m, n′, and n″. These helical conformations include 3₁₀-helices (e.g., m=0 and n′+n″=2), α-helices (e.g., m=1 and n′+n″=2), π-helices (e.g., m=2 and n′+n″=2), and gramicidin helices (e.g., m=4 and n′+n″=2). In a preferred embodiment, the number of atoms in the backbone of the helical macrocycle is 12-15, more preferably 13 or 14.

In at least one embodiment of compounds of Formula I, m′″ is one and a is two.

In at least one embodiment, R² is: a beta amino acid, a moiety of Formula A where m′ is at least one and at least one b is two, a moiety of Formula A where c is two, or a moiety of Formula A where R^{2′} is a beta amino acid. In at least one embodiment, R³ is: a beta amino acid, a moiety of Formula B where m″ is at least one and at least one d is two, or a moiety of Formula B where R^{3′} is a beta amino acid. Combinations of these embodiments are also contemplated.

When R² is a moiety of Formula A, m′ is preferably any number from one to 19. When R³ is a moiety of Formula B, m″ is preferably any number from one to nine.

In preferred embodiments, the compound is a compound of Formula IA, Formula IIA, or Formula IIIA (i.e., a helix cyclized at the N-terminal); Formula IB, Formula IIB, or Formula IIIB (i.e., a helix cyclized mid-peptide); or Formula IC, Formula IIC, or Formula IIIC (i.e., a helix cyclized at the C-terminal):

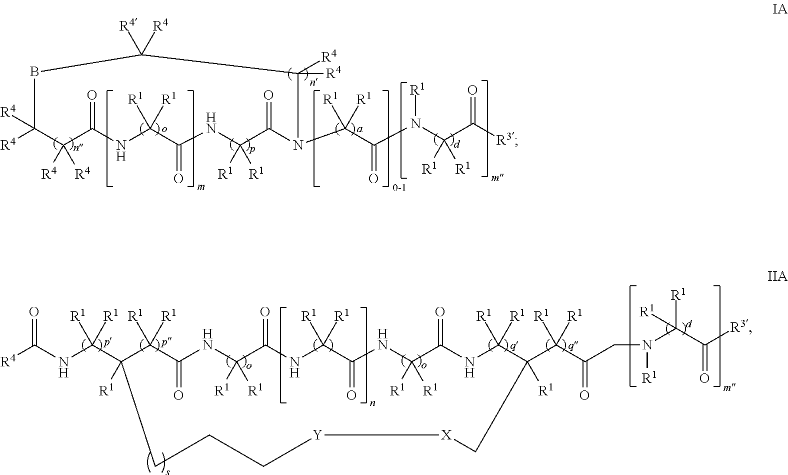

where R⁴ is independently hydrogen, an alkyl, an alkenyl, an alkynyl, a cycloalkyl, a heterocyclyl, an aryl, a heteroaryl, or an arylalkyl;

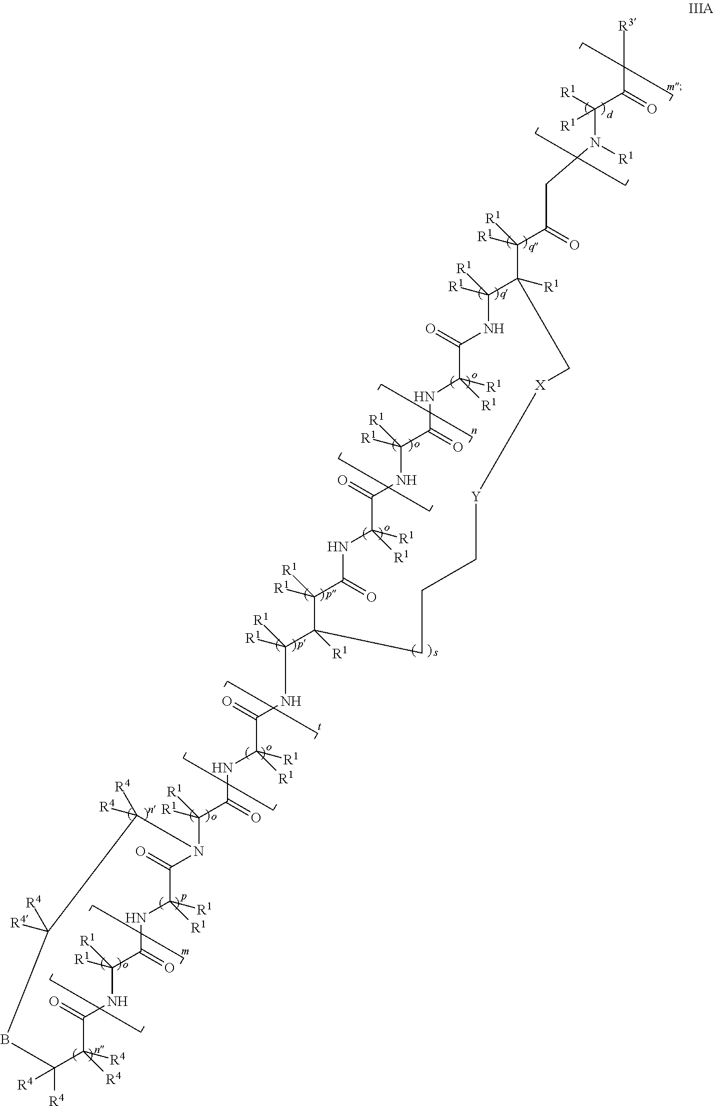

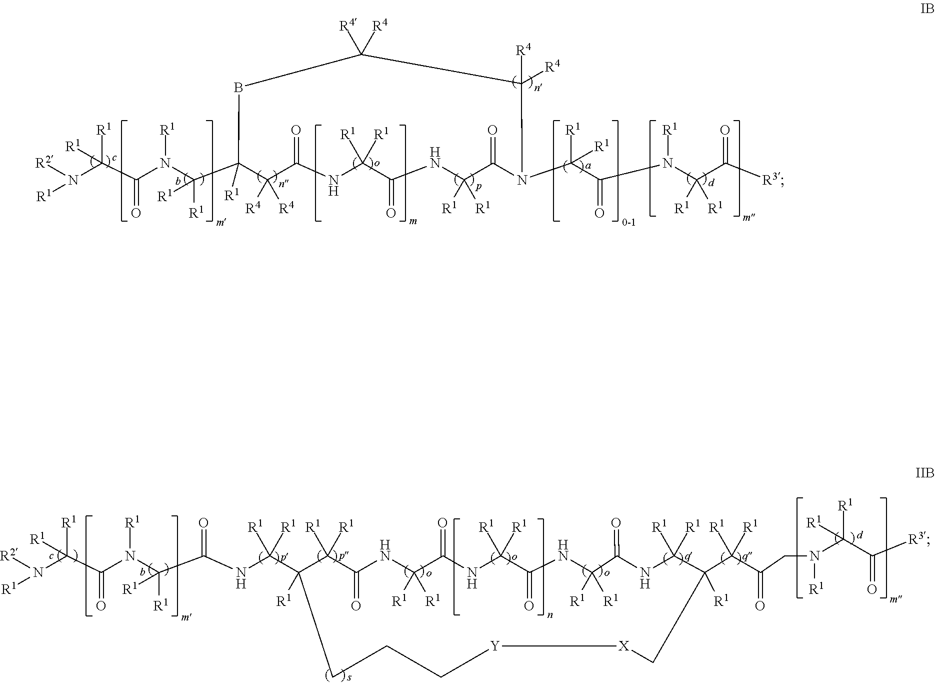

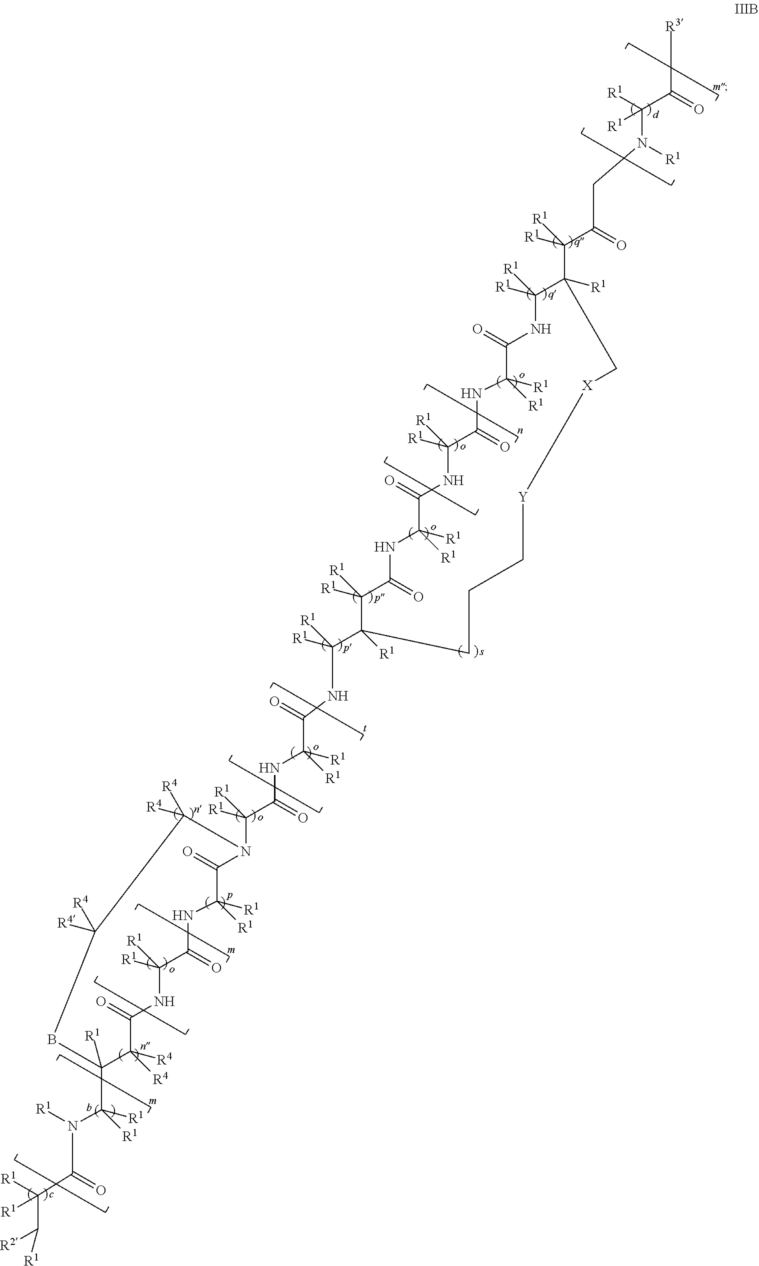

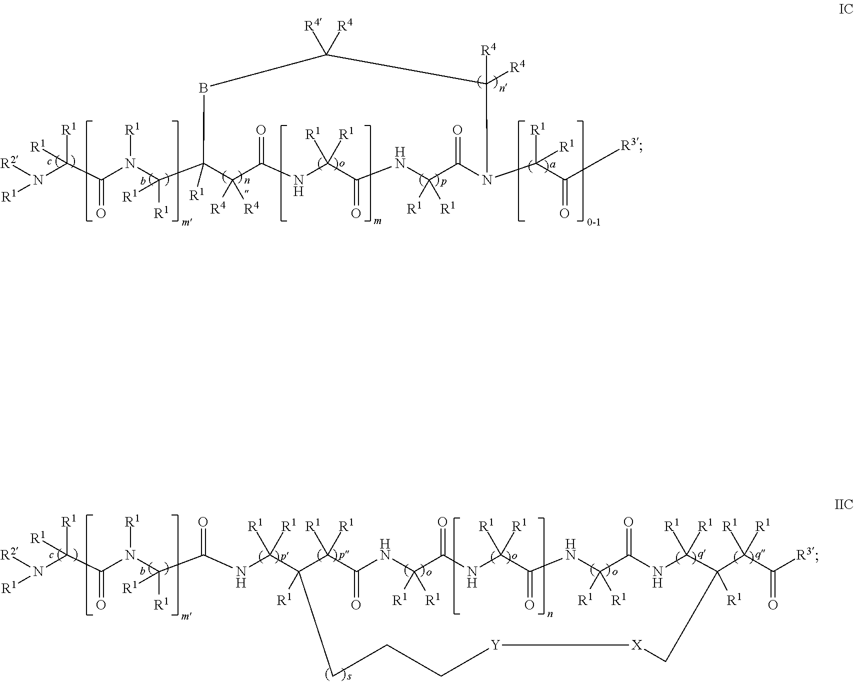

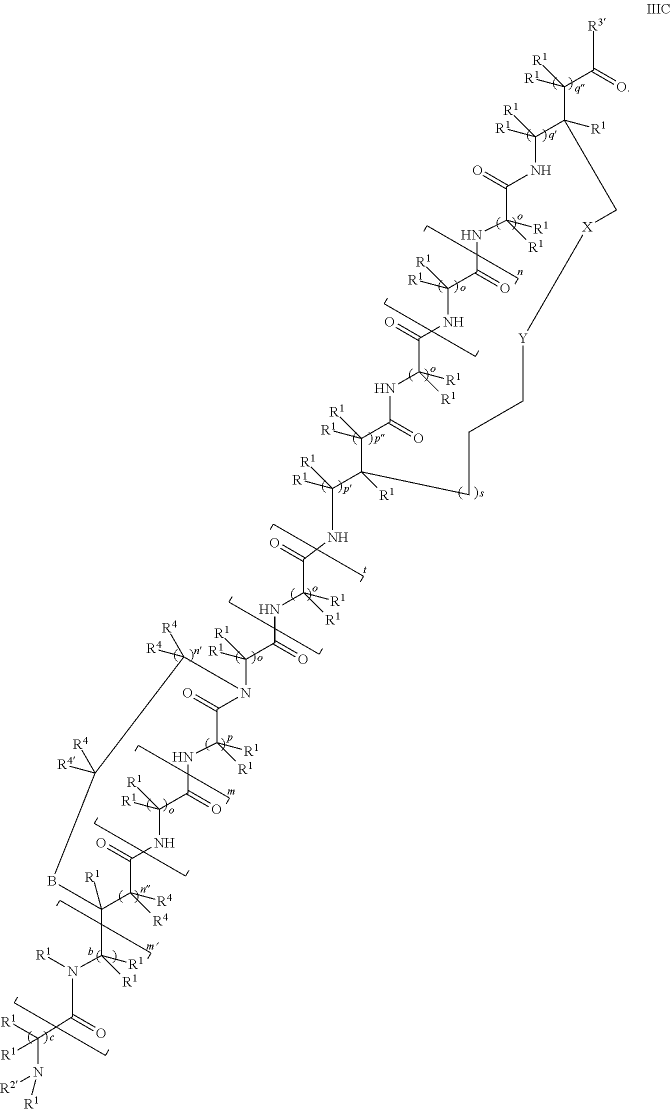

As will be apparent to the skilled artisan, the pattern of β substitution in the attached peptides of the peptidomimetics of Formulae I, II, and III can be controlled by adjusting the values for m′″ and a (when the peptidomimetic is a compound of Formula I), as well as m′, b, and c (when R² is a moiety of Formula A), and m″ and d (when R³ is a moiety of Formula B). Substitution in peptidomimetics of Formulae IA, HA, IIIA, IB, IIB, IIIB, IC, IIC, and IIIC can further be controlled as will be apparent to the skilled artisan. In a preferred embodiment, the attached peptide has the formula α3/β1. Preferred peptidomimetics containing β-amino acid residues include those that mimic a helix having the formula X₁-x₂-X₂—X₃—X₂—X₂—X₁—X₄-x₅, wherein X₅ is absent or any hydrophobic residue and the beta residues are shown in lower-case bold. Preferred embodiments include, without limitation, XeEGRaLDQ (SEQ ID NO: 10), XeLLRaLDQ (SEQ ID NO: 11), XeLARaLDQ (SEQ ID NO: 12), and XeEGRaLDQy (SEQ ID NO: 13).

The peptidomimetics of the present invention may be prepared using methods that are known in the art. By way of example, peptidomimetics of Formula I, which contain a hydrogen bond surrogate, may be prepared using the methods disclosed in, e.g., U.S. patent application Ser. No. 11/128,722, U.S. patent application Ser. No. 13/724,887, and Mahon & Arora, “Design, Synthesis, and Protein-Targeting Properties of Thioether-Linked Hydrogen Bond Surrogate Helices,” Chem. Commun. 48:1416-18 (2012), each of which is hereby incorporated by reference in its entirety. Peptidomimetics of Formula II, which contain a side-chain constraint, may be prepared using the methods disclosed in, e.g., Schafmeister et al., J. Am. Chem. Soc. 122:5891 (2000); Sawada & Gellman, J. Am. Chem. Soc. 133:7336 (2011); Patgiri et al., J. Am. Chem. Soc. 134:11495 (2012); Henchey et al., Curr. Opin. Chem. Biol. 12:692 (2008); Harrison et al., Proc. Nat'l Acad. Sci. U.S.A. 107:11686 (2010); Shepherd et al., J. Am. Chem. Soc. 127:2974 (2005); Phelan et al., J. Am. Chem. Soc. 119:455 (1997); Jackson et al., J. Am. Chem. Soc. 113:9391 (1991); and Blackwell & Grubbs, Angew. Chem. Intl Ed. Engl. 37:3281 (1998), each of which is hereby incorporated by reference in its entirety. Peptidomimetics of Formula III, which contain both a hydrogen bond surrogate and a side-chain constraint, may be prepared using a combination of the above methods.

Another aspect of the present invention relates to pharmaceutical formulations comprising any of the above described peptidomimetics of Formula I, Formula II, or Formula III of the present invention (including the peptidomimetics of Formulae IA, IIA, IIIA, IB, IIB, IIIB, IC, IIC, and IIIC) and a pharmaceutically acceptable carrier. Acceptable pharmaceutical carriers include solutions, suspensions, emulsions, excipients, powders, or stabilizers. The carrier should be suitable for the desired mode of delivery.

In addition, the pharmaceutical formulations of the present invention may further comprise one or more pharmaceutically acceptable diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The peptidomimetics and pharmaceutical formulations of the present invention may be used, inter alia, to inhibit the HIF-1α-p300/CBP interaction.

Another aspect of the present invention relates to a method of modulating transcription of a gene in a cell, wherein transcription of the gene is mediated by interaction of HIF-1α with CBP and/or p300. This method involves contacting the cell with a peptidomimetic of the present invention under conditions effective to modulate transcription of the gene. In a preferred embodiment, the cell is contacted under conditions effective to cause nuclear uptake of the peptide, where the peptide disrupts interaction of HIF-1α and p300/CBP and thereby reduces transcription of the gene.

Modulating according to this aspect of the present invention refers to up-regulating transcription or down-regulating transcription.

Genes whose transcription can be modulated according to this aspect of the present invention include ACADSB, ADM, AK4, ALDOC, ALG1, ANG, ANGPTL4, ANKRD37, ANKZF 1, ARHGAP28, ARID5A, ARNTL, ARRDC3, ASF1A, ASPM, AURKA, B4GALT4, BAMBI, BHLHE40, BHLHE41, BNIP3, BNIP3L, BOLA1, C1orf161, C1orf163, C3orf58, C4orf3, C7orf60, C7orf68, C8orf22, C8orf41, C14orf126, C17orf76, C18orf19, C1QL1, CA12, CA5B, CA9, CASZ1, CCDC80, CCNB1, CCNG2, CDC20, CDC23, CDCP1, CDK18, CDKN1A, CDKN3, CENPA, CENPE, CGGBP1, CHAC2, CNOT8, CPOX, CXCL16, CXCR4, DAPK1, DDX10, DEPDC1, DIS3L, DKFZp451A211, DLGAP5, DUSP5, DUSP5P, DUSP9, E2F5, EDN2, EFNA3, EGLN1, EGLN3, ELOVL6, ENO2, ERO1L, ERRFI1, FAM13A, FAM72A, FAM72B, FAM72C, FAM72D, FAM83D, FAM86B1, FAM86B2, FAM86C, FAM115C, FAM115C, FAM133A, FAM162A, FARSB, FBXO16, FBXO32, FBXO42, FERMT1, FLJ23867, FLJ35024, FLJ44715, FM, FOS, FOXD1, FUT11, FXYD3, FYN, G2E3, GBE1, GDF15, GEMIN5, GFPT2, GOLGA8A, GOLGA8B, GPATCH4, GPR146, GPR155, GPR160, GPRC5A, GPT2, GTF2IRD2, GTF2IRD2B, GYS1, H1F0, H2BFS, HAS2, HERC3, HEY1, HIST1H1C, HIST1H1E, HIST1H2AB, HIST1H2AC, HIST1H2AD, HIST1H2AE, HIST1H2AH, HIST1H2AI, HIST1H2AK, HIST1H2AL, HIST1H2BC, HIST1H2BE, HIST1H2BF, HIST1H2BG, HIST1H2BH, HIST1H2BI, HIST1H2BJ, HIST1H2BK, HIST1H2BM, HIST1H2BN, HIST1H3A, HIST1H3D, HIST1H3F, HIST1H3H, HIST1H4B, HIST1H4H, HIST1H4J, HIST1H4K, HIST2H2AA3, HIST2H2AA4, HIST2H2AB, HIST2H2AC, HIST2H2BA, HIST2H2BE, HIST2H2BF, HIST2H3A, HIST2H3C, HIST2H3D, HIST2H4A, HIST2H4B, HIST3H2A, HIVEP2, HK1, HK2, HMMR, HORMAD1, HOXD10, HPDL, HRH1, HSPA1A, HSPA1B, HYMAI, ID3, IDH2, IER3, IGFBP3, IGSF3, IL1RAP, IL2RG, ING2, INSIG1, INSIG2, IPMK, ITGA5, JUN, KAT2B, KCTD11, KDM3A, KIAA0586, KIAA1244, KIAA1432, KIAA1715, KIF14, KIF20A, KRT17, LOC154761, LOC645332, LOC653113, LOC100507405, LOX, LOXL2, LRP1, LST-3TM12, LTV1, MAFB, MAFK, MAK16, MAP2K1, MAP3K15, METTL7A, MLKL, MOBKL2A, MSTO1, MSTO2P, MUC1, MXI1, NAMPT, NARS2, NAV1, NDRG1, NDUFAF4, NEBL, NFIL3, NLN, NOG, NOL6, NOP2, NOP16, NOTCH3, NRG4, ORAI3, OSMR, OTUD1, P4HA1, P4HA2, PAG1, PAIP2B, PDHA1, PDK1, PDK3, PER1, PER2, PFKFB4, PFKP, PGM2L1, PIAS2, PLA2G4A, PLAGL1, PLIN2, PLK1, PLOD1, PLOD2, PMEPA1, PNO1, POLR1B, PPFIA4, PPL, PPP1R3B, PPP1R3C, PPP2R5B, PPRC1, PRELID2, PRMT3, PTGS2, PTTG1, PYGL, QSOX1, RAB20, RAB40C, RAB8B, RASSF2, RCOR2, RIOK3, RIT1, RLF, RNASE4, RNF 122, RNF24, RNU4-2, RORA, RPSA, RRAGD, RRS1, RUVBL1, SCARNA5, SCARNA6, SCFD2, SEC14L4, SEC61G, SERPINE1, SERPINI1, SERTAD2, SLC2A1, SLC2A3, SLC6A10P, SLC6A6, SLC6A8, SLC7A11, SLC27A2, SLCO1B3, SLCO4A1, SNAPC5, SNORA1, SNORA2A, SNORA6, SNORA13, SNORA42, SNORA60, SNORA62, SNORA74A, SNORA75, SNORD1A, SNORD14E, SNORD53, SNORD94, SNX33, SPAG4, SPICE1, SPINK5, SPRY1, STAMBPL1, STC2, SYT7, TAF9B, TBC1D30, TCP11L2, TET2, TGFB1, TMCO7, TMEM45A, TMEM45B, TMEM184A, TMOD1, TMPRSS3, TNFRSF 10D, TRIM59, TROAP, TSEN2, TSTD2, TTYH3, TWISTNB, UACA, UBASH3B, UFSP2, UPRT, UTP15, UTP20, VEGFA, VLDLR, VTRNA1-1, WDR3, WDR12, WDR35, WDR45L, WDR52, WSB1, XK, YEATS2, ZDBF2, ZNF 160, ZNF292, ZNF395, ZNF654, ZSWIM5, adenylate kinase 3, α_{1B}-adrenergic receptor, aldolase A, ceruloplasmin, c-Met protooncogene, CXCL12/SDF-1, endothelin-1, enolase 1, erythropoietin, glucose transporter 1, glucose transporter 3, glyceraldehyde-3-phosphate dehydrogenase, heme oxygenase 1, IGF binding protein 1, insulin-like growth factor 2, lactate dehydrogenase A, nitric oxide synthase 2, p35^{srg}, phosphoglycerate kinase 1, pyruvate kinase M, transferrin, tranferrin receptor, transforming growth factor β₃, vascular endothelial growth factor, vascular endothelial growth factor receptor FLT-1, and vascular endothelial growth factor receptor KDR/Flk-1. Some uses for inhibiting transcription of some of these genes are shown in Table 1. Preferred genes include those identified in Table 5, infra.

TABLE 1
Example Disorders

| Gene | Treat/prevent |
|---|---|
| adrenomedullin | Pheochromocytoma |
| ceruloplasmin | Lymphoma, acute and chronic |
| | inflammation, |
| | rheumatoid arthritis |
| c-Met protooncogene | Tumor Cells Invasion |
| CXCL12/SDF-1 | Cancer Stem Cells Migration |
| CXCR4 | Cancer Stem Cells Migration |
| endothelin-1 | Abnormal vasoconstriction |
| endothelin-2 | Abnormal vasoconstriction |
| enolase 1 | Hashimoto's encephalopathy, |
| | severe asthma |
| erythropoietin | Abnormal oxygen transport |
| glucose transporter 1 | Aerobic glycolysis (Warburg effect) |
| glucose transporter 3 | Aerobic glycolysis (Warburg effect) |
| heme oxygenase 1 | Abnormal oxygen transport |
| hexokinase 1 | Aerobic glycolysis (Warburg effect) |
| hexokinase 2 | Aerobic glycolysis (Warburg effect) |
| IGF binding protein 1 | Abnormal development and function of |
| | organs (brain, liver) |
| IGF binding protein 3 | Abnormal development and function of |
| | organs (brain, liver) |
| insulin-like growth factor 2 | Abnormal development and function of |
| | organs (brain, liver) |
| lactate dehydrogenase A | Myocardial infarction |
| lysyl oxidase | Tumor Cells Invasion |
| nitric oxide synthase 2 | Abnormal vasomotor tone |
| tranferrin receptor | Abnormal iron uptake/metabolism |
| transferrin | Abnormal iron uptake/metabolism |
| vascular endothelial growth factor | Angiogenesis (tumor, incl. cancer) |
| vascular endothelial growth factor | Angiogenesis (tumor, incl. cancer) |
| receptor FLT-1 | |
| vascular endothelial growth factor | Angiogenesis (tumor, incl. cancer) |
| receptor KDR/Flk-1 | |

Yet another aspect of the present invention relates to a method of treating or preventing in a subject a disorder mediated by interaction of HIF-1α with CBP and/or p300. This method involves administering to the subject a peptidomimetic of the present invention under conditions effective to treat or prevent the disorder.

Disorders that can be treated or prevented include, for example, abnormal vasoconstriction, retinal ischemia (Zhu et al., “Long-Term Tolerance to Retinal Ischemia by Repetitive Hypoxic Preconditioning: Role of HIF-1α and Heme Oxygenase-1,” Invest. Ophthalmol. Vis. Sci. 48:1735-43 (2007); Ding et al., “Retinal Disease in Mice Lacking Hypoxia-Inducible Transcription Factor-2α,” Invest. Ophthalmol. Vis. Sci. 46:1010-16 (2005), each of which is hereby incorporated by reference in its entirety), pulmonary hypertension (Simon et al., “Hypoxia-Induced Signaling in the Cardiovascular System,” Annu. Rev. Physiol. 70:51-71 (2008); Eul et al., “Impact of HIF-1α and HIF-2α on Proliferation and Migration of Human Pulmonary Artery Fibroblasts in Hypoxia,” FASEB J. 20:163-65 (2006), each of which is hereby incorporated by reference in its entirety), intrauterine growth retardation (Caramelo et al., “Respuesta a la Hipoxia. Un Mecanismo Sistémico Basado en el Control de la Expresión Génica [Response to Hypoxia. A Systemic Mechanism Based on the Control of Gene Expression],” Medicina B. Aires 66:155-64 (2006); Tazuke et al., “Hypoxia Stimulates Insulin-Like Growth Factor Binding Protein 1 (IGFBP-1) Gene Expression in HepG2 Cells: A Possible Model for IGFBP-1 Expression in Fetal Hypoxia,” Proc. Nat'l Acad. Sci. USA 95:10188-93 (1998), each of which is hereby incorporated by reference in its entirety), diabetic retinopathy (Ritter et al., “Myeloid Progenitors Differentiate into Microglia and Promote Vascular Repair in a Model of Ischemic Retinopathy,” J. Clin. Invest. 116:3266-76 (2006); Wilkinson-Berka et al., “The Role of Growth Hormone, Insulin-Like Growth Factor and Somatostatin in Diabetic Retinopathy,” Curr. Med. Chem. 13:3307-17 (2006); Vinores et al., “Implication of the Hypoxia Response Element of the Vegf Promoter in Mouse Models of Retinal and Choroidal Neovascularization, but not Retinal Vascular Development,” J. Cell. Physiol. 206:749-58 (2006); Caldwell et al., “Vascular Endothelial Growth Factor and Diabetic Retinopathy: Role of Oxidative Stress,” Curr. Drug Targets 6:511-24 (2005), each of which is hereby incorporated by reference in its entirety), age-Related macular degeneration (Inoue et al., “Expression of Hypoxia-Inducible Factor 1α and 2α in Choroidal Neovascular Membranes Associated with Age-Related Macular Degeneration,” Br. J. Ophthalmol. 91:1720-21 (2007); Zuluaga et al., “Synergies of VEGF Inhibition and Photodynamic Therapy in the Treatment of Age-Related Macular Degeneration,” Invest. Ophthalmol. Vis. Sci. 48:1767-72 (2007); Provis, “Development of the Primate Retinal Vasculature,” Prog. Retin. Eye Res. 20:799-821 (2001), each of which is hereby incorporated by reference in its entirety), diabetic macular edema (Vinores et al., “Implication of the Hypoxia Response Element of the Vegf Promoter in Mouse Models of Retinal and Choroidal Neovascularization, but not Retinal Vascular Development,” J. Cell. Physiol. 206:749-58 (2006); Forooghian & Das, “Anti-Angiogenic Effects of Ribonucleic Acid Interference Targeting Vascular Endothelial Growth Factor and Hypoxia-Inducible Factor-1α,” Am. J. Ophthalmol. 144:761-68 (2007), each of which is hereby incorporated by reference in its entirety), and cancer (Marignol et al., “Hypoxia in Prostate Cancer: A Powerful Shield Against Tumour Destruction?” Cancer Treat. Rev. 34:313-27 (2008); Galanis et al., “Reactive Oxygen Species and HIF-1 Signalling in Cancer,” Cancer Lett. 266:12-20 (2008); Ushio-Fukai & Nakamura, “Reactive Oxygen Species and Angiogenesis: NADPH Oxidase as Target for Cancer Therapy,” Cancer Lett. 266:37-52 (2008); Adamski et al., “The Cellular Adaptations to Hypoxia as Novel Therapeutic Targets in Childhood Cancer,” Cancer Treat. Rev. 34:231-46 (2008); Toffoli & Michiels, “Intermittent Hypoxia Is a Key Regulator of Cancer Cell and Endothelial Cell Interplay in Tumours,” FEBS J. 275:2991-3002 (2008), each of which is hereby incorporated by reference in its entirety).

The subject according to this aspect of the present invention is preferably a human subject.

The compounds of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Another aspect of the present invention relates to a method of reducing or preventing angiogenesis in a tissue. This method involves contacting the tissue with a peptidomimetic of the present invention under conditions effective to reduce or prevent angiogenesis in the tissue.

Preferred tissues according to this aspect of the present invention include tumors.

Yet another aspect of the present invention relates to a method of decreasing survival and/or proliferation of a cell under hypoxic conditions. This method involves contacting the cell with a peptidomimetic of the present invention under conditions effective to decrease survival and/or proliferation of the cell.

Suitable cells according to this and all aspects of the present invention include, without limitation, mammalian cells. Preferably, the cells are human cells. In at least one embodiment, the cells are cancer cells or are contained in the endothelial vasculature of a tissue that contains cancerous cells. Suitable cancer cells include, e.g., sarcoma cells, multiple myeloma cells, prostate cancer cells, melanoma cells, brain cancer cells, ovarian cancer cells, breast cancer cells, renal cancer cells, and eye cancer cells.

In all aspects of the present invention directed to methods involving contacting a cell with one or more peptidomimetics, contacting can be carried out using methods that will be apparent to the skilled artisan, and can be done in vitro or in vivo.

One approach for delivering agents into cells involves the use of liposomes. Basically, this involves providing a liposome which includes agent(s) to be delivered, and then contacting the target cell, tissue, or organ with the liposomes under conditions effective for delivery of the agent into the cell, tissue, or organ.

This liposome delivery system can also be made to accumulate at a target organ, tissue, or cell via active targeting (e.g., by incorporating an antibody or hormone on the surface of the liposomal vehicle). This can be achieved according to known methods.

An alternative approach for delivery of protein- or polypeptide-containing agents (e.g., peptidomimetics of the present invention containing one or more protein or polypeptide side chains) involves the conjugation of the desired agent to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of agents involves preparation of chimeric agents according to U.S. Pat. No. 5,817,789 to Heartlein et al., which is hereby incorporated by reference in its entirety. The chimeric agent can include a ligand domain and the agent (e.g., a peptidomimetic of the invention). The ligand domain is specific for receptors located on a target cell. Thus, when the chimeric agent is delivered intravenously or otherwise introduced into blood or lymph, the chimeric agent will adsorb to the targeted cell, and the targeted cell will internalize the chimeric agent.

Peptidomimetics of the present invention may be delivered directly to the targeted cell/tissue/organ.

Additionally and/or alternatively, the peptidomimetics may be administered to a non-targeted area along with one or more agents that facilitate migration of the peptidomimetics to (and/or uptake by) a targeted tissue, organ, or cell. As will be apparent to one of ordinary skill in the art, the peptidomimetic itself can be modified to facilitate its transport to a target tissue, organ, or cell, including its transport across the blood-brain barrier; and/or to facilitate its uptake by a target cell (e.g., its transport across cell membranes). In a preferred embodiment, the peptide of the invention is modified, and/or delivered with an appropriate vehicle, to facilitate its delivery to the nucleus of the target cell (Wender et al., “The Design, Synthesis, and Evaluation of Molecules That Enable or Enhance Cellular Uptake: Peptoid Molecular Transporters,” Proc. Nat'l Acad. Sci. USA 97:13003-08 (2000); Roberts, “Buyer's Guide to Protein Transduction Reagents,” Scientist 18:42-43 (2004); Joliot & Prochiantz, “Transduction Peptides: From Technology to Physiology,” Nat. Cell Biol. 6:189-96 (2004), each of which is hereby incorporated by reference in its entirety). Some example target cells, tissues, and/or organs for the embodiments described above are shown in Table 2.

TABLE 2
Example Target Cells/Tissues/Organs

| Desired Effect | Example Target(s) |
|---|---|
| Inhibit transcription of: | |
| enolase 1 | Liver, brain, kidney, spleen, adipose, |
| | lung |
| glucose transporter 1 | Tumor, incl. cancer |
| glucose transporter 3 | Tumor, incl. cancer |
| hexokinase 1 | Tumor, incl. cancer |
| hexokinase 2 | Tumor, incl. cancer |
| insulin-like growth factor 2 | Brain, liver |
| IGF binding protein 1 | Brain, liver |
| IGF binding protein 3 | Brain, liver |
| lactate dehydrogenase A | Heart |
| ceruloplasmin | Lymphocytes/lymphatic tissue, |
| | inflamed tissue, rheumatoid arthritic |
| | tissue |
| erythropoietin | Liver, kidney |
| transferrin | Liver |
| adrenomedullin | Pheochromocytoma |
| endothelin-1 | Endothelium |
| nitric oxide synthase 2 | Vessels, cardiovascular cells/tissue |
| vascular endothelial growth factor | Tumor cells/tissue, incl. cancer |
| vascular endothelial growth factor | Tumor cells/tissue, incl. cancer |
| receptor FLT-1 | |
| vascular endothelial growth factor | Tumor cells/tissue, incl. cancer |
| receptor KDR/Flk-1 | |

Treat or prevent:

| | |
|---|---|
| retinal ischemia | Retina (eye) |
| pulmonary hypertension | Lungs |
| intrauterine growth retardation | Uterus |
| diabetic retinopathy | Retina (eye) |
| age-related macular degeneration | Retina (eye) |
| diabetic macular edema | Retina (eye) |
| Reduce or prevent angiogenesis | Tumor cells/tissue, incl. cancer |
| Decrease cell survival and/or | Cancerous cells, cells contained in the |
| proliferation | endothelial vasculature of a tissue that |
| | contains cancerous cells |

In vivo administration can be accomplished either via systemic administration to the subject or via targeted administration to affected tissues, organs, and/or cells, as described above. Typically, the therapeutic agent (i.e., a peptidomimetic of the present invention) will be administered to a patient in a vehicle that delivers the therapeutic agent(s) to the target cell, tissue, or organ. Typically, the therapeutic agent will be administered as a pharmaceutical formulation, such as those described above.

Exemplary routes of administration include, without limitation, orally, topically, transdermally, parenterally, subcutaneously, intravenously, intramuscularly, intraperitoneally, intraventricularly, and intralesionally; by intratracheal inoculation, aspiration, airway instillation, aerosolization, nebulization, intranasal instillation, oral or nasogastric instillation, intraperitoneal injection, intravascular injection, intravenous injection, intra-arterial injection (such as via the pulmonary artery), intramuscular injection, and intrapleural instillation; by application to mucous membranes (such as that of the nose, throat, bronchial tubes, genitals, and/or anus); and by implantation of a sustained release vehicle.

For use as aerosols, a peptidomimetic of the present invention in solution or suspension may be packaged in a pressurized aerosol container together with suitable propellants, for example, hydrocarbon propellants like propane, butane, or isobutane with conventional adjuvants. The peptidomimetics of the present invention also may be administered in a non-pressurized form.

Exemplary delivery devices include, without limitation, nebulizers, atomizers, liposomes (including both active and passive drug delivery techniques) (Wang & Huang, “pH-Sensitive Immunoliposomes Mediate Target-Cell-Specific Delivery and Controlled Expression of a Foreign Gene in Mouse,” Proc. Nat'l Acad. Sci. USA 84:7851-55 (1987); Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; U.S. Pat. No. 5,059,421 to Loughrey et al.; Wolff et al., “The Use of Monoclonal Anti-Thyl IgG1 for the Targeting of Liposomes to AKR-A Cells in Vitro and in Vivo,” Biochim. Biophys. Acta 802:259-73 (1984), each of which is hereby incorporated by reference in its entirety), transdermal patches, implants, implantable or injectable protein depot compositions, and syringes. Other delivery systems which are known to those of skill in the art can also be employed to achieve the desired delivery of the peptidomimetic to the desired organ, tissue, or cells in vivo to effect this aspect of the present invention.

Contacting (including in vivo administration) can be carried out as frequently as required and for a duration that is suitable to provide the desired effect. For example, contacting can be carried out once or multiple times, and in vivo administration can be carried out with a single sustained-release dosage formulation or with multiple (e.g., daily) doses.

The amount to be administered will, of course, vary depending upon the particular conditions and treatment regimen. The amount/dose required to obtain the desired effect may vary depending on the agent, formulation, cell type, culture conditions (for ex vivo embodiments), the duration for which treatment is desired, and, for in vivo embodiments, the individual to whom the agent is administered.

Effective amounts can be determined empirically by those of skill in the art. For example, this may involve assays in which varying amounts of the peptidomimetic of the invention are administered to cells in culture and the concentration effective for obtaining the desired result is calculated. Determination of effective amounts for in vivo administration may also involve in vitro assays in which varying doses of agent are administered to cells in culture and the concentration of agent effective for achieving the desired result is determined in order to calculate the concentration required in vivo. Effective amounts may also be based on in vivo animal studies.

Another aspect of the present invention relates to a method of identifying an agent that potentially inhibits interaction of HIF-1α with CBP and/or p300. This method involves providing a peptidomimetic of the present invention, contacting the peptidomimetic with a test agent, and detecting whether the test agent selectively binds to the peptidomimetic, wherein a test agent that selectively binds to the peptidomimetic is identified as a potential inhibitor of interaction between HIF-1α with CBP and/or p300.

This aspect of the present invention can be carried out in a variety of ways, that will be apparent to the skilled artisan. For example, the affinity of the test agent for the peptidomimetic of the present invention may be measured using isothermal titration calorimetry analysis (Wiseman et al., “Rapid Measurement of Binding Constants and Heats of Binding Using a New Titration calorimeter,” Anal. Biochem. 179:131-37 (1989); Freire et al., “Isothermal Titration calorimetry,” Anal. Chem. 62:A950-A959 (1990); Chervenak & Toone, “Calorimetric Analysis of the Binding of Lectins with Overlapping Carbohydrate-Binding Ligand Specificities,” Biochemistry 34:5685-95 (1995); Aki et al., “Competitive Binding of Drugs to the Multiple Binding Sites on Human Serum Albumin. A calorimetric Study,” J. Thermal Anal. Calorim. 57:361-70 (1999); Graziano et al., “Linkage of Proton Binding to the Thermal Unfolding of Sso7d from the Hyperthermophilic Archaebacterium Sulfolobus solfataricus,” Int' J. Biol. Macromolecules 26:45-53 (1999); Pluschke & Mutz, “Use of Isothermal Titration calorimetry in the Development of Molecularly Defined Vaccines,” J. Thermal Anal. Calorim. 57:377-88 (1999); Corbell et al., “A Comparison of Biological and calorimetric Analyses of Multivalent Glycodendrimer Ligands for Concanavalin A,” Tetrahedron-Asymmetry 11:95-111 (2000), which are hereby incorporated by reference in their entirety). In one embodiment, a test agent is identified as a potential inhibitor of interaction between HIF-1α with CBP and/or p300 if the dissociation constant (K_{d}) for the test agent and the peptidomimetic of the invention is 50 μM or less. In another embodiment, the K_{d} is 200 nM or less. In another embodiment, the K_{d} is 100 nM or less.

Test agents identified as potential inhibitors of HIF-1α-p300/CREB interaction may be subjected to further testing to confirm their ability to inhibit interaction between HIF-1α with CBP and/or p300.

The present invention may be further illustrated by reference to the following examples.

### EXAMPLES

The following Examples are intended to illustrate, but by no means are intended to limit, the scope of the present invention as set forth in the appended claims.

### Example 1

### General Materials and Methods

Commercial grade solvents and reagents were used without further purification. Fmoc amino acids and peptide synthesis reagents were purchased from Novabiochem. Hoveyda-Grubbs (second-generation) catalyst was obtained from Sigma. Molecular biology grade salts and buffers were obtained from Sigma. Cell culture media and reagents were purchased from Invitrogen, unless otherwise stated.

### Peptide Synthesis

Peptides were synthesized on a CEM Liberty series microwave peptide synthesizer and purified by reversed-phase HPLC. The identity and purity of the peptides were confirmed by LCMS (see Table 3 below).

TABLE 3
Mass Spectroscopic Characterization of
HBS Helices and Peptide 3

| | | Calculated | Observed |
|---|---|---|---|
| Compound | Sequence^{a} | [M + H]⁺ | [M + H]⁺ |
| HBS 1 | XELA*RALDQ-NH₂ | 1008.5 | 1008.5 |
| | (SEQ ID NO: 14) | | |
| HBS 2 | XELA*RAADQ-NH₂ | 966.5 | 966.5 |
| | (SEQ ID NO: 15) | | |
| Peptide 3 | AcELARALDQ-NH₂ | 956.5 | 956.5 |
| | (SEQ ID NO: 16) | | |

^{a}X denotes 4-pentenoic acid; A* = N-allylalanine.

### Synthesis of HBS Peptides

HBS helices containing only α-amino acid residues were synthesized as previously described (Patgiri et al., “Solid-Phase Synthesis of Short α-Helices Stabilized by the Hydrogen Bond Surrogate Approach,” Nat. Protoc. 5(10):1857-65 (2010), which is hereby incorporated by reference in its entirety) (see Scheme 1 below).

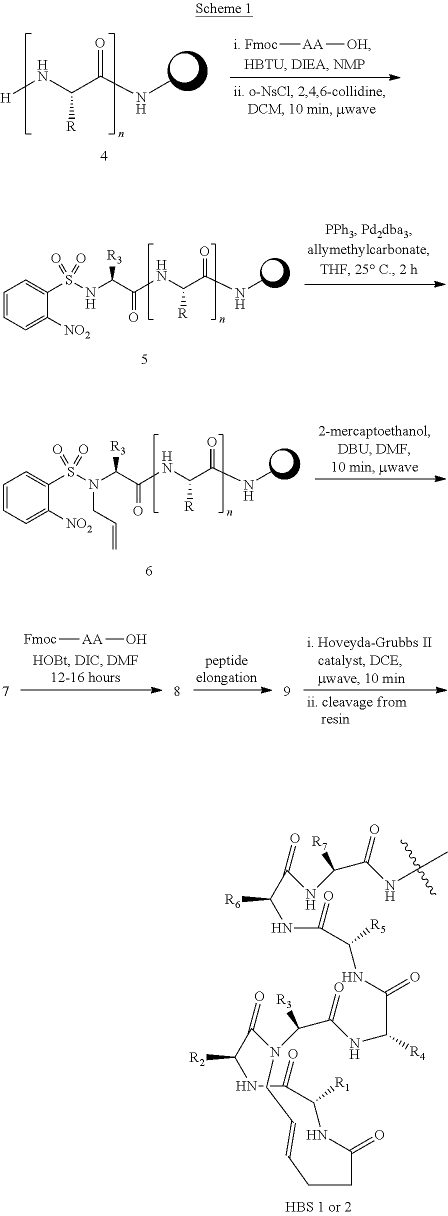

Peptide sequences up to the i+3^{rd} residue of the putative helix (4 in Scheme 1) were synthesized on solid phase on a CEM Liberty Series microwave peptide synthesizer. A solution containing premixed o-nitrobenzesulfonyl chloride (10 eq) and 2,4,6-collidine (10 eq) in DCM was added to Fmoc-deprotected, resin bound 4. Resin was washed sequentially with DCM (×3), DMF (×3), DCM (×3), and diethyl ether. Resin was dried overnight under vacuum. Dried resin, PPh₃, and Pd₂(dba)₃ were flushed with argon for 30 minutes. Upon addition of THF, allymethylcarbonate was added to the reaction vessel containing dissolved reactants and resin. The solution was agitated at room temperature for 3 to 5 hours under argon to afford 5.

Resin was filtered and washed with DCM (×3), DMF (×3), 0.2 M sodium diethylcarbamate trihydrate in NMP, and diethyl ether. The nosyl protecting group was then removed by the addition of 1,8-diazabicyclo[5.4.0]undec-7-ene (DBU, 5eq) and 2-mercaptoethanol (10 eq.) in DMF. Resin was washed with DMF (×3), DCM (×3), and diethyl ether and treated with the desired Fmoc amino acid (20 eq.), DIC (20 eq.), and HOAt (10 eq.) in DMF and was allowed to agitate at room temperature for 12 to 16 hours.

Resin containing 8 was then washed with DMF (×3), DCM (×3), and DMF (×3), and coupled to the desired Fmoc amino acid residue (5 eq.) and 4-pentenoic acid (5 eq.) with HBTU (5 eq.) and DIEA (10 eq.) in DMF.

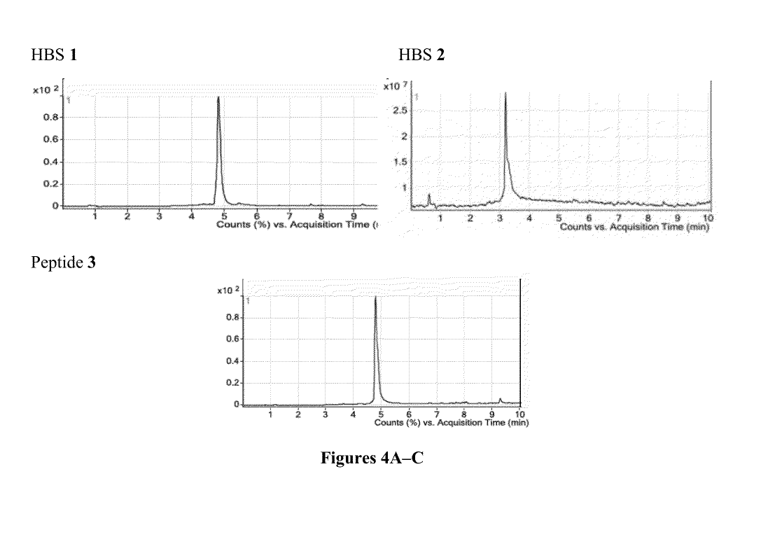

Ring-closing metathesis of bis-olefin 9 was performed with Hoveyda-Grubbs II catalyst (20 mol %) in 1,2-dichloroethane under microwave irradiation at 120° C. for 10 minutes as described in Patgiri et al., “Solid-Phase Synthesis of Short α-Helices Stabilized by the Hydrogen Bond Surrogate Approach,” Nat. Protoc. 5(10):1857-65 (2010); Chapman & Arora, “Optimized Synthesis of Hydrogen-Bond Surrogate Helices: Surprising Effects of Microwave Heating on the Activity of Grubbs Catalysts,” Org. Lett. 8(25):5825-28 (2006); and Patgiri et al., “Solid Phase Synthesis of Hydrogen Bond Surrogate Derived Alpha-Helices: Resolving the Case of a Difficult Amide Coupling,” Org. Biomol. Chem. 8:1773-76 (2010), each of which is hereby incorporated by reference in its entirety. Peptides were cleaved from the resin using TFA:TIS:water (95:2.5:2.5), and purified by reversed-phase HPLC (C₁₈ column) in 0.1% TFA acetonitrile/water gradients and characterized by ESI-MS. The computational alanine scanning mutagenesis energies calculated with Rosetta ver. 3.3. are shown in Table 4 below. Scans were performed on the HIF-1α/CBP complex (PDB codes 1L8C and 1L3E). Peptides were also analyzed by HPLC (see FIGS. 4A-C).

TABLE 4
Computational Alanine Scanning Mutagenesis Energies
HELIX B (817-824): ELLRALDQ (SEQ ID NO: 17)

| Residue | Helix B residue | ΔΔG (kcal/mol) |
|---|---|---|
| Leu | 818 | 1.4 |
| Leu | 819 | 0.5 |
| Arg | 820 | 0.1 |
| Ala | 821 | 0.0 |
| Leu | 82 | 21.9 |
| Asp | 823 | 1.4 |
| Gln | 824 | 0.3 |

An HBS helix containing β-amino acid residues (i.e., XeEG*RaLDQ-NH₂ (SEQ ID NO: 18), bold lower case letters denote β-residues) was synthesized as previously described with the necessary modification (Patgiri et al., “Solid-Phase Synthesis of Short α-Helices Stabilized by the Hydrogen Bond Surrogate Approach,” Nat. Protoc. 5(10):1857-65 (2010); Patgiri et al., “Nucleation Effects in Peptide Foldamers,” J. Am. Chem. Soc. 134(28):11495-502 (2012), each of which is hereby incorporated by reference in its entirety) (see Scheme 2 below).

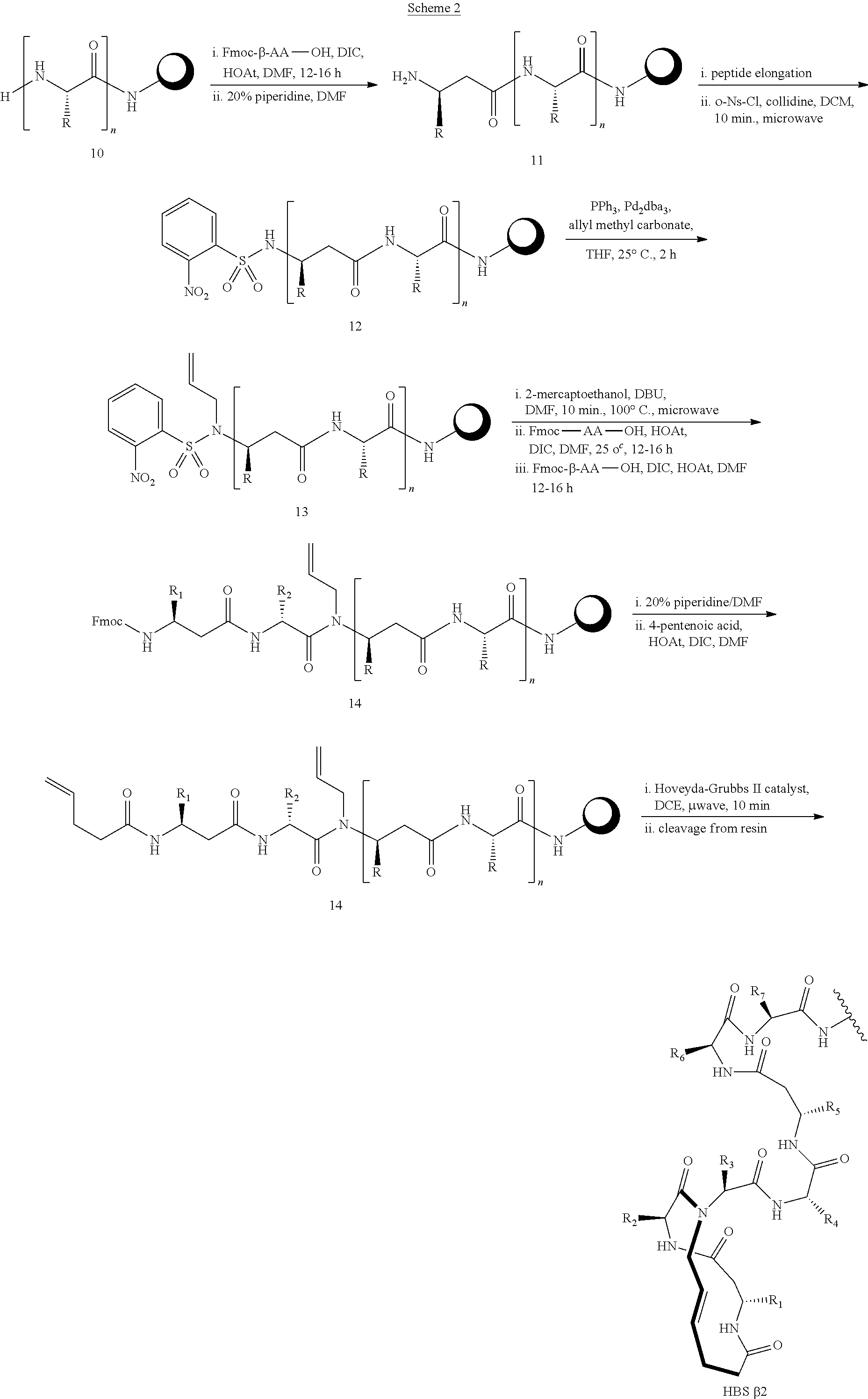

The peptide sequence up to the putative helix 10 in Scheme 2 was synthesized on solid phase via a CEM Liberty Series microwave peptide synthesizer or by hand. A solution containing premixed β-Fmoc amino acid (20 eq.), DIC (20 eq.), and HOAt (10 eq.) in DMF was added to Fmoc-deprotected resin bound 10 at room temperature for 12 to 16 hours. Resin was washed sequentially with DCM (×3), DMF (×3), and MeOH (×3) to afford 11.

After Fmoc-deprotection and two further α-amino acid peptide elongation, a solution containing premixed o-nitrobenzesulfonyl chloride (10 eq) and 2,4,6-collidine (10 eq) in DCM was added to Fmoc-deprotected, resin bound 11. Resin was washed sequentially with DCM (×3), DMF (×3), DCM (×3), and diethyl ether to afford 12.

Resin bound 12 was dried overnight under vacuum, then PPh₃, and Pd₂(dba)₃ were added and flushed with argon for 30 minutes. Upon addition of THF, allymethylcarbonate was added to the reaction vessel containing dissolved reactants and resin. The solution was agitated at room temperature for 3 to 5 hours under argon to afford 13.

Resin was filtered and washed with DCM (×3), DMF (×3), 0.2 M sodium diethylcarbamate trihydrate in NMP, and diethyl ether. The nosyl protecting group was then removed by the addition of 1,8-diazabicyclo[5.4.0]undec-7-ene (DBU, 5eq) and 2-mercaptoethanol (10 eq.) in DMF. Resin was washed with DMF (×3), DCM (×3), and diethyl ether and treated with the desired Fmoc amino acid (20 eq.), DIC (20 eq.), and HOAt (10 eq.) in DMF and was allowed to agitate at room temperature for 12 to 16 hours.

Resin containing 14 was then washed with DMF (×3), DCM (×3), and MeOH (×3), and coupled to the desired β-Fmoc amino acid residue (5 eq.). Use of 4-pentenoic acid (5 eq.) DIC (20 eq.), and HOAt (10 eq.) in DMF afforded 15.

Ring-closing metathesis of bis-olefin 15 was performed with Hoveyda-Grubbs II catalyst (20 mol %) in 1,2-dichloroethane under microwave irradiation at 120° C. for 10 minutes as described in Patgiri et al., “Solid-Phase Synthesis of Short α-Helices Stabilized by the Hydrogen Bond Surrogate Approach,” Nat. Protoc. 5(10):1857-65 (2010); Patgiri et al., “Nucleation Effects in Peptide Foldamers,” J. Am. Chem. Soc. 134(28):11495-502 (2012); Chapman & Arora, “Optimized Synthesis of Hydrogen-Bond Surrogate Helices: Surprising Effects of Microwave Heating on the Activity of Grubbs Catalysts,” Org. Lett. 8(25):5825-28 (2006); and Patgiri et al., “Solid Phase Synthesis of Hydrogen Bond Surrogate Derived Alpha-Helices: Resolving the Case of a Difficult Amide Coupling,” Org. Biomol. Chem. 8:1773-76 (2010), each of which is hereby incorporated by reference in its entirety. Peptides were cleaved from the resin using TFA:TIS:water (95:2.5:2.5), and purified by reversed-phase HPLC (C₁₈ column) in 0.1% TFA acetonitrile/water gradients and characterized by ESI-MS.

Additional mimics containing beta amino acids, including XeLL*RaLDQ-NH₂ (SEQ ID NO: 19), XeLA*RaLDQ-NH₂ (SEQ ID NO: 20), XeEG*RaLDQy-NH₂ (SEQ ID NO: 21), will also be synthesized. The β-residue-containing mimics are expected to be more resistant to degradation than their α-amino acid counterparts.

### Circular Dichroism Studies

CD spectra were recorded on an AVIV 202SF CD spectrometer equipped with a temperature controller using 1 mm length cells and a scan speed of 0.5 nm/min at 298K. The spectra were averaged over 10 scans with the baseline subtracted from analogous conditions as those for the samples. The samples were prepared in 10 mM. KF with the final peptide concentration of 50 μM.

### Plasmids

The DNA sequence of human p300 CH1 domain (amino acid residues 323-423) was designed as an insert and subcloned into a pUC57 plasmid by Genscript, Inc. After transformation of the plasmid in JM109 bacteria (Promega), the gene sequence was subcloned into BamHI and EcoRI restriction sites of pGEX-4T-2 expression vector (Amersham).

Cloning and Expression of ¹⁵N p300-CH1

The pGEX 4T-2-p300 fusion vector was transformed into BL21 (DE3)-competent E. coli (Novagen) in M9 minimal media with ¹⁵NH₄Cl as the main nitrogen source. Protein production was induced with 1 mM IPTG at O.D. 600 of 1 for 16 hours at 15° C. Production of the desired p300-CH1-GST fusion product was verified by SDS-PAGE. Bacteria were harvested and resuspended in the lysis buffer with 20 mM Phosphate buffer (Research Products International, Corp.), 100 μM DTT (Fisher), 100 μM ZnSO₄ (Sigma), 0.5% TritonX 100 (Sigma), 1 mg/mL Pepstatin A (Research Products International, Corp.), 10 mg/mL Leupeptin A (Research Products International, Corp.), 500 μM PMSF (Sigma), and 0.5% glycerol at pH 8.0. Pellets were lysed by sonication and centrifuged at 4° C., 20,000 rpm, for 20 minutes. Fusion protein was collected from the bacterial supernatant and purified by affinity chromatography using glutathione Sepharose 4B beads (Amersham) prepared according to the manufacturer's directions. GST-tag was cleaved by thrombin and protein was eluted from resin. Collected fractions were assayed by SDS-PAGE gel; pooled fractions were treated with protease inhibitor cocktail (Sigma) and against a buffer containing 10 mM Tris, 50 mM NaCl, 2 mM DTT (Fisher), and 3 equivalents ZnSO₄ at pH 8.0 to ensure proper folding (vide supra).

### Tryptophan Fluorescence Binding Assay

Spectra were recorded on a QuantaMaster 40 spectrofluorometer (Photon Technology International) in a 10 mm quartz fluorometer cell at 25° C. with 4 nm excitation and 4 nm emission slit widths from 200 to 400 nm at intervals of 1 nm/s. Samples were excited at 295 nm and fluorescence emission was measured from 200-400 nm and recorded at 335 nm. Peptide stock solutions were prepared in DMSO. Aliquots containing 1 μL DMSO stocks were added to 400 μL of 1 μM p300-CH1 in 50 mM Tris and 100 mM NaCl (pH 8.0). After each addition, the sample was allowed to equilibrate for 5 minutes before UV analysis. Background absorbance and sample dilution effects were corrected by titrating DMSO into p300-CH1 in an analogous manner. Final fluorescence is reported as the absolute value of [(F₁−F₀)/F₁]*100, where F₁ is the final fluorescence upon titration and F₀ is the fluorescence of the blank DMSO titration. EC₅₀ values for each peptide were determined by fitting the experimental data to a sigmoidal dose-response nonlinear regression model on GraphPad Prism 5.0, and the dissociation constants, K_{D}, were obtained from equation (1)

K_{D}=(EC₅₀×(1−F)+P×F²)/F−P (1)
P=Total concentration of protein
F=Fraction of bound peptide=0.5

### Fluorescence Polarization Assay

The relative affinity of peptides for ¹⁵N-labeled p300-CH1 was determined using fluorescence polarization binding assay with fluoresceine-tagged HIF-1αC-TAD₇₈₆₋₈₂₆. The polarization experiments were performed with a DTX 880 Multimode Detector (Beckman) at 25° C., with excitation and emission wavelengths of 485 and 525 nm, respectively. Addition of an increasing concentration (0 nm to 13.5 μM) of p300-CH1 protein to a 15 nM solution of fluorescein labeled HIF peptide in 20 mM Tris pH 8.0, 50 mM NaCl, 2 mM DTT, 3 eq ZnSO₄, and 0.1% pluronic F-68 (Sigma) in 96 well plates afforded the IC₅₀ value, which was fit into equation (2) to calculate the dissociation constant (K_{D}) for the HIF/p300 complex (Roehrl et al., “A General Framework for Development and Data Analysis of Competitive High-Throughput Screens for Small-Molecule Inhibitors of Protein-Protein Interactions by Fluorescence Polarization,” Biochemistry 43(51):16056-66 (2004), which is hereby incorporated by reference in its entirety).

K_{D}=(R_{T}×(1−F_{SB})+L_{ST}×F_{SB}²)/F_{SB}−L_{ST} (2)
R_{T}=Total concentration of p300-CH1 protein
L_{ST}=Total concentration of fluorescent peptide
F_{SB}=Fraction of bound fluorescent peptide

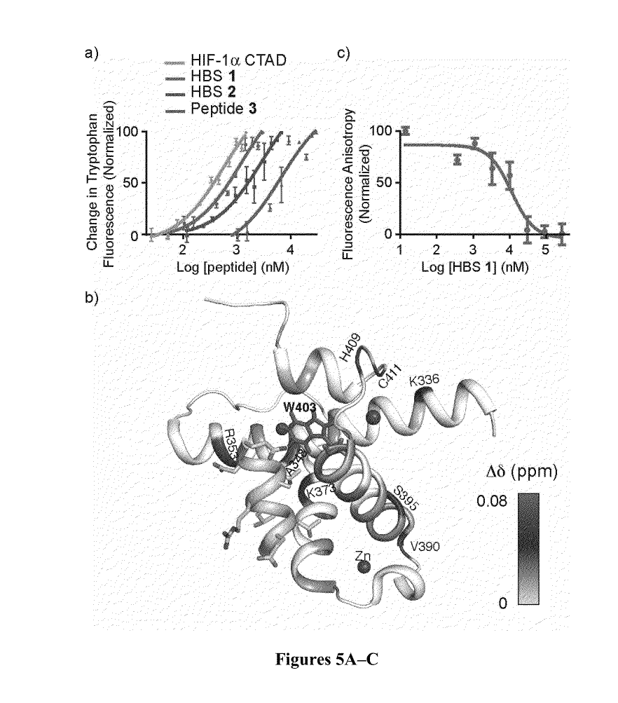

The binding affinity (K_{D}) reported for each peptide is the average of three individual experiments, and was determined by fitting the experimental data to a sigmoidal dose-response nonlinear regression model on GraphPad Prism 5.0. The K_{D} of Flu-HIF C-TAD was determined to be 31±3 nM. For competitive inhibition experiments, a solution of 300 nM p300-CH1 and 15 nM Flu-HIF C-TAD in buffer (20 mM Tris (pH 8.0), 50 mM NaCl, 2 mM DTT, and 150 μM ZnSO₄) and 0.1% pluronic acid was incubated at 25° C. in a 96 well plate. After 30 minutes, appropriate concentrations of the HBS or linear peptides were added to the p300-CH1/Flu-HIF C-TAD solution and the resulting mixtures were incubated at 25° C. for 30 minutes before measuring the degree of dissociation of Flu-HIF C-TAD by polarization. The EC₅₀ was fit into equation (3) to calculate the K, value of HBS 1. The inhibition curve is shown in FIG. 5C.

Kᵢ=K_{D1}*F_{SB}*((L_{T}/L_{ST}*F_{SB2}−(K_{D1}+L_{ST}+R_{T})*F_{SB}+R_{T}))−1/(1−F_{SB})) (3)
K_{D}=K_{D} of fluorescent probe Flu-HIF C-TAD
R_{T}=Total concentration of p300-CH1 protein
L_{ST}=Total concentration of HIF fluorescent peptide
F_{SB}=Fraction of bound HBS 1 (at EC₅₀)
L_{T}=Total concentration of HBS 1 (EC₅₀)

### ¹H-¹⁵N HSQC NMR Spectroscopy

Protein samples were prepared as described above. Uniformly ¹⁵N-labelled p300-CH1 was concentrated to 69 μM in NMR buffer (10 mM Tris pH 8, 50 mM NaCl, 2 mM DTT, and 207 μM ZnSO₄) using a 3 kDa MWCO Amicon Ultra centrifugal filter (Millipore) and supplemented with 5% D₂O. For HSQC titration experiments, data was collected on a 600 MHz Bruker four-channel NMR system at 25° C. and analyzed with the TopSpin software (Bruker). For Zn²⁺ experiments, data were collected on Agilent 600 MHz at 25° C. and analyzed using Sparky3 (Univ. of California).

For the HSQC titration experiments, five and ten molar equivalents of HBS 1 in DMSO were added to ¹⁵N-labelled p300-CH1, and the data were collected as described above. Mean chemical shift difference (Δδ_{NH}) observed for ¹H and ¹⁵N nuclei of various resonances were calculated as described in Williamson, “Using Chemical Shift Perturbation to Characterise Ligand Binding,” Prog. Nucl. Mag. Resonance Spectr. 73(0):1-16 (2013), which is hereby incorporated by reference in its entirety, where a is the range of H ppm shifts divided by the range of NH ppm shifts (α=⅛).

d=\sqrt{1/2[δ_{H}²+(α·δ_{N}²)]}

### Cell Lines and Cell Culture

Human cervical epithelial adenocarcinoma (HeLa) and human renal cell carcinoma (786-0) cell lines were obtained from ATCC. Aggressive human breast carcinoma stably transfected with an HRE luciferase construct (MDA-MB-231-HRE-Luc) was a gift of Dr. Robert Gillies. HeLa cells were grown at 37° C. in a humidified atmosphere with 5% CO₂ in high glucose Dulbecco's Modified Eagle's Medium (DMEM, Sigma) supplemented with 10%, 2%, or 02% of fetal bovine serum (FBS, Irvine Scientific) and 0.5% Pen-Strep (Sigma). MDA-MB-231-HRE-Luc cells were grown in high glucose DMEM supplemented with 10% fetal bovine serum and 0.4 g/L geneticin (RPI). Hypoxia was mimicked with desferrioxamine mesylate (DFO, Sigma) at a concentration of 300 μM or by GasPak EZ pouch (BD Biosciences). Cell growth and morphology were monitored by phase-contrast microscopy.

Isolation of mRNA

HeLa cells (˜70% confluent) were plated in 6-well dishes (BD Falcon) at a density of 1.5×10⁵ cells/mL. After attachment, cells were treated with 1.5 mL of fresh media containing HBS 1, HBS 2, and peptide 1 at concentrations of 10 μM and 50 μM. All samples, including vehicle, contained a final concentration of 0.1% DMSO. After 6 hours, hypoxia was induced with DFO (300 μM) or GasPak EZ pouch and cells were incubated for another 18 and 42 hours, respectively. Cells were lysed and RNA isolated according to the protocol described in Dubey et al., “Suppression of Tumor Growth by Designed Dimeric Epidithiodiketopiperazine Targeting Hypoxia-Inducible Transcription Factor Complex,” J. Am. Chem. Soc. 135(11):4537-49 (2013), which is hereby incorporated by reference in its entirety.

### Analysis of Gene Expression

Real-time qRT-PCR was used to determine the effect of HBS 1, HBS 2, and peptide 1 on VEGF, LOX, and SLC2A1 (GLUT1) genes in the HeLa cell lines, as described in Dubey et al., “Suppression of Tumor Growth by Designed Dimeric Epidithiodiketopiperazine Targeting Hypoxia-Inducible Transcription Factor Complex,” J. Am. Chem. Soc. 135(11):4537-49 (2013), which is hereby incorporated by reference in its entirety. Statistical analyses were performed with data from four independent replicates.

### Cell Viability Assays

HeLa cells were plated in a 96-well plate at a density of 6,000 cells/well and allowed to form a monolayer before adding the compounds. After attachment, the media was replaced by 100 μL of fresh media containing HBS 1, HBS 2, or peptide 1 at a concentration ranging from 1 μM to 100 μM, and 0.1% DMSO as a vehicle. After 24 hours of incubation with compounds, 11 μL of 3-(4,5 -dimethylthiazolyl-2)-2,5-diphenyltetrazolium bromide (MTT, Sigma) at a concentration of 5 mg/mL in PBS was added to each well and incubated at 37° C. and 5% CO₂ for an additional 3 hours. After 3 hours of incubation, the media was removed and purple crystals were dissolved in 100 μL of dimethyl sulfoxide (DMSO). The absorbance was measured at 570 nm with a correction at 690 nm in order to quantify the amount of formed purple formazan. All experiments were performed in quadruplicate.

### Luciferase Assays

MDA-MB-231-HRE-Luc cells were plated in 24-well plates (BD Falcon) at a seeding density of 35,000 cells/mL. Cells were allowed to adhere and form a monolayer before adding the compounds (˜70% confluence). After attachment, cells were treated with 1 mL of fresh media containing HBS 1, HBS 2, or peptide 1 at a concentration of 10 μM and 50 μM. All samples contained a final concentration of 0.1% DMSO; vehicle samples were treated with cell culture media containing 0.1% DMSO. Cells were incubated for 6 hours at 37° C. and 5% CO₂ and then hypoxia was induced by placing the cells into GasPak EZ pouch for another 18 hours. The lysates were isolated by using cell culture lysis reagent (Promega). Prior to collecting cell lysate, halt protease inhibitor cocktail (Thermo Scientific) was added to the cell culture lysis reagent in order to ensure the stability of proteins. Cell lysates were collected into low-adhesion pre-chilled Eppendorf tubes (USA Scientific) and centrifuged at 13,000 rpm for 5 minutes at 4° C. Supernatant was collected into another set of pre-chilled Eppendorf tubes and the pellet was discarded. Luciferase assay reagent (100 μL, Promega) was added to 20 μL of cell lysate and luminescence intensity was measured by Turner TD-20e luminometer. The results were normalized to total protein concentration determined by BCA assay. Briefly, 10 μL of cell lysate was added to 200 μL of BCA reagent. Absorbance was measured at 562 nm using a BioTek Synergy 2 microplate reader and normalized to BSA solutions at a concentration range of 125 μg/mL to 2000 μg/mL as standards.

Determination of Protein Levels with ELISA

Cells were plated in 24-well dishes (BD-Falcon) at a density of 35,000 cells/mL. Cells were allowed to attach overnight (˜70% confluent) before dosing with the compound. After 24 hours, the old media was replaced with fresh media containing 2% FBS, and HBS 1 at concentrations ranging from 1 μM to 10 μM. All samples contained a final concentration of 0.1% DMSO; vehicle samples were treated with cell culture media containing 0.1% DMSO. Cells were incubated tier 6 hours at 37° C. and 5% CO₂ and hypoxia was induced with DFO (300 μM), and cells were incubated for another 18 hours. The supernatant was collected and the levels of VEGF were measured with the Human Quantikine VEGF kit (R&D Systems) in accordance with the manufacturer's protocol. Absorbance was measured at 450 nm on a BioTek Synergy 2 microplate reader. The readings were normalized to a total protein concentration. Every experiment was performed in quadruplicate.

### Western Blot Analysis of HIF-1α Levels

HeLa cells were plated in a 75 cm² culture flask and allowed to reach 70% confluence. Cells were treated with vehicle or HBS 1 at 10 μM concentration in the cell culture media containing 10% FBS. AU samples contained a final concentration of 0.1% DMSO. Cells were incubated for 6 hours and hypoxia was induced with 300 μM. DFO. After incubation for an additional 18 hours, cells were lysed and cytoplasmic and nuclear extracts were collected using a NE-PER kit (Pierce) according to the manufacturer's protocol and blotted as described in Dubey et al., “Suppression of Tumor Growth by Designed Dimeric Epidithiodiketopiperazine Targeting Hypoxia-Inducible Transcription Factor Complex,” J. Am. Chem. Soc. 135(11):4537-49 (2013), which is hereby incorporated by reference in its entirety.

### Plasma Stability and Biodistribution Studies

Plasma stability and biodistribution studies were performed in 10-week-old female BALB/c mice (Charles River) with 3 mice per time point. Briefly, HBS 1 or peptide 3 was dissolved in 70 μL of sterile PBS and administered intravenously at a dose of 1 mg/kg. Then, 1 mL of blood was collected by cardiac puncture at euthanasia at the following time points: 30 min., 1 h., 2 h., 4 h., 6 h., 8 h., 12 h., 16 h., and 24 h. after drug administration. The experiments were performed under an approved IACUC protocol at the University of Southern California.

Samples were prepared by mixing 30 μL of plasma with 20 μL of 50% MeOH and 50% aqueous 1% formic acid. The mixture was vortexed and mixed with an additional 120 μL of 0.5% formic acid in MeOH/ACN (4:6) and 20 μL of 2.0 μg/mL isoproterenol in MeOH/1% aqueous formic acid (1:1) as an internal standard. The mixture was vortexed again for 2 minutes and centrifuged at 13,000 rpm for 4 minutes. Next, 20 μL of the supernatant was transferred to a new tube and mixed with 180 μL of 50% MeOH/ACN (4:6) and 50% aqueous 1% formic acid. Standard curves were prepared by mixing the plasma from three untreated mice with 20 μL of 50% MeOH and 50% aqueous 1% formic acid prepared with HBS 1 or peptide 3 at a concentration range of 0.05-2 μg/mL. The standard curves, as determined by linear regression, displayed good linearity (r²>0.98) over the range tested.

Samples were analyzed by LC/MS/MS using an Agilent 6210 time-of-flight LC/MS system. HPLC separation was achieved using a Prevail 3u C₁₈ 100×2.1 mm column (Grace Davison, Deerfield, Ill., USA). The column temperature was maintained at 20° C. The mobile phase consisted of A (5% acetonitrile and 95% of 0.05% aqueous formic acid) and B (5% of 05% aqueous formic acid and 95% acetonitrile). The following gradient program was used: 0% B (0 min, 0.125 ml/min), 100% B (17 min, 0.125 ml/min). The total run time was 35 minutes. The electrospray ionization source of the mass spectrometer was operated in positive ion mode with the capillary voltage set to 4 kV, and the cone and collision cell voltages optimized to 60 and 170 V. The source temperature was 120° C. and the desolvation temperature was 300° C. A solvent delay/divert program was used from 0 to 4.0 minutes to minimize the mobile phase to flow to the source. Agilent MassHunter Workstation version B.02.01 software was used for data acquisition and processing.

### Gene Expression Profiling

Experiments were carried out with HeLa cells. The media, time course, DFO, and small molecule treatments were the same as for the qRT-PCR assays. Cultured cells contained vehicle, HBS 1, or HBS 2 at a concentration of 50 μM. RNA was isolated as previously described. Sample preparation and microarray analysis was performed at the Genome Technology Center, New York University School of Medicine. Labeled mRNA was hybridized to Affymetrix Genechip Human Gene 1.0 ST microarrays. Four data sets were collected: normoxic cells with vehicle, hypoxic cells with vehicle, hypoxic cells with HBS 1, and hypoxic cells with HBS 2. Gene expression profiles were analyzed using GeneSpring GX 12.5 software (Agilent). Probe level data have been converted to expression values using a robust multi-array average (RMA) preprocessing procedure on the core probe sets and baseline transformation to median of all samples. A low-level filter removed the lowest 20^{th} percentile of all the intensity values and generated a profile plot of filtered entities. Significance analysis was performed by one-way ANOVA test with Benjamini-Hochberg correction and asymptotic P-value computation. Fold change analysis was applied to identify genes with expression ratios above 1.1-fold between treatments and control set (P<0.05). Hierarchical agglomerative clustering was performed using Pearson's centered correlation coefficient and average-linkage as distance and linkage methods. The gene expression profiling data have been deposited in the Gene Expression Omnibus (GEO) database, www.ncbi.nlm.nih.gov/GEO (accession no. GSE48002).

### In Vivo Efficacy Tests of HBS 1 in Mouse Xenograft Tumor Models

CrTac:NCr-Foxn1^{nu} mice (Taconic, Inc.) were used to examine the in vivo efficacy of HBS 1. Mice were housed in an A.L.A.C.C. approved barrier facility under the direct supervision of a professional veterinarian. Mice (n=6) were inoculated with 786-O cells (2×10⁶ cells) into the right flank and allowed to grow tumors for 21 days. The primary endpoint of efficacy (the rate of increase in tumor volume as compared to control) were evaluated when mice were treated with HBS 1 at 13 mg/kg dissolved in sterile PBS given parenterally on days 4, 7, 11, 25, and 28, a total of 5 injections. In parallel, a control group (n=6) received injections of PBS. Tumor sizes were measured on Days 2, 3, 4, 6, 8, 11, 13, 16, 20, 25, 28, and 33. To address the question of whether tumor growth is affected by treatment with HBS, a comparison of the tumor volumes of the control group and the group treated with HBS 1 was made. At a conclusion of the study, mice were injected intraperitoneally with the near-infrared dye IR-783 contrast agent and the tumors were imaged using Xenogen IVIS 200 small animal imager. Euthanasia was performed as recommended by the American Veterinary Panel (AVMA 202229-249, 1993). The organs and tumors were collected for future histopathology studies.

### Example 2

### Design and Synthesis of Stabilized α-Helices

HIF-1α forms a heterodimer with its β subunit, aryl hydrocarbon receptor nuclear translocator (ARNT), to recognize hypoxia response element (HRE) and up-regulate expression of hypoxia-inducible genes, which are important contributors to tumor progression. Pyrrole-imidazole polyamides, which are programmable DNA-binding small molecules, have been shown to regulate transcription of hypoxia-inducible genes by binding to the HRE. Initiation of HIF-mediated transcription also requires complex formation between the CH1 domain of the coactivator protein p300 (or the homologous CREB binding protein, CBP) and the C-TAD₇₈₆₋₈₂₆ of HIF-1α(FIG. 3A). This transcription factor-coactivator interaction represents an alternative target for controlling hypoxia signaling. Structural studies provide a molecular basis for this interaction and identify two short α-helical domains, αA and αB, from HIF-1α as key determinants for its recognition by p300 (FIG. 3C). Both αA and αB subdomains of HIF-1αC-TAD contain residues that contribute significantly to the complex formation, as shown by experimental mutagenesis studies. In earlier work, the αA peptide sequence was stabilized using the hydrogen bond surrogate (HBS) approach, which utilizes a carbon-carbon bond in place of the intramolecular hydrogen bond in α-helices. HBS helices have been shown to disrupt intracellular protein-protein interactions with high affinity and specificity. The αA mimetic was shown to downregulate mRNA levels of VEGF and GLUT1, two genes under the control of HIF-1α, while the linear peptide mimic of αA remained inactive. Importantly, the compound did not display significant toxicity as compared to chetomin, a small molecule known to target the same interaction. As described herein, the ability of αB mimics to inhibit the target interaction and control gene expression in cell culture was explored and its efficacy was tested in murine tumor xenograft models.

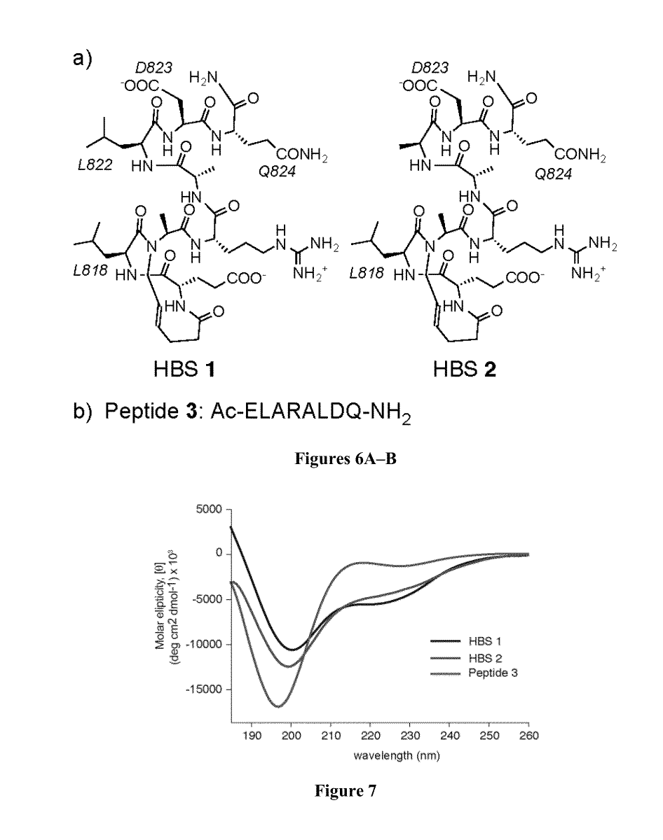

A key premise of rational design is that, unlike high throughput screening efforts, a handful of molecules that fit certain criteria need to be designed de novo. In an ideal scenario, these predictions would lead to both a potent ligand for the target receptor and a compound serving as a negative control, featuring minor alterations and binding the same protein with reduced affinity. Such a result would confirm the fundamental design principles while allowing the specificity of designed compounds to be evaluated. Accordingly, two stabilized helices based on the wild-type sequence were conceived (FIG. 6A), along with the unconstrained control (FIG. 6B).

HBS 1 is a direct mimic of HIF-1α₈₁₇₋₈₂₄ with the exception of Leu819, which was changed to an alanine residue to streamline synthetic effort (coupling of an N-alkyl alanine to the next residue is more efficient than coupling N-alkyl leucine). Computational alanine scanning mutagenesis analysis suggests that Leu819 is not a significant contributor to binding energy as opposed to Leu818, Leu822, Asp823, and Gln824 (see Table 4, supra).

HBS 2 was designed to be a specificity control in which the critical Leu-822 residue is replaced with an alanine; based on computational data, HBS 2 would be expected to bind CH1 with an order of magnitude weaker binding affinity than HBS 1.

Peptide 3 is an unconstrained analog of HBS 1; allowing the effect of helix stabilization on the activity of the compounds to be evaluated. The HBS helices were synthesized, purified, and characterized by HPLC and circular dichroism spectroscopy, as described above. As shown in FIG. 7, The constrained peptides showed characteristic α-helical circular dichroism spectroscopy signatures in aqueous buffers as compared to the unconstrained derivative, which displays no discernible helicity, as expected for a very short peptide.

### Example 3

### Designed Ligands Target p300-CH1 in a Predictive Manner

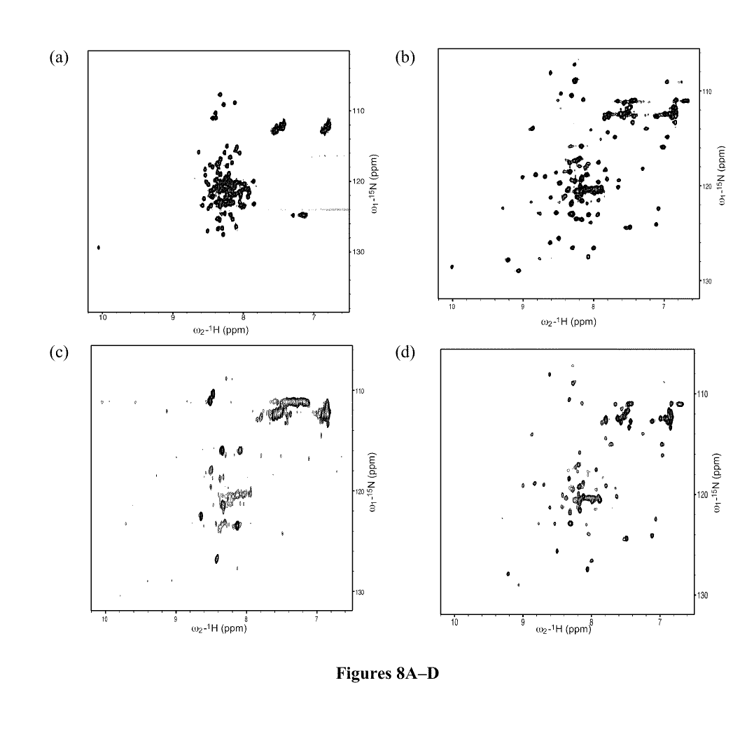

The CH1 domain of p300/CBP is stabilized by three zinc ions. Prior NMR structural studies have shown that the purified protein can rapidly aggregate in a buffer with excess or deficiency in Zn²⁺ (Patgiri et al., “A Hydrogen Bond Surrogate Approach for Stabilization of Short Peptide Sequences in Alpha-Helical Conformation,” Acc. Chem. Res. 41(10):1289-300 (2008), which is hereby incorporated by reference in its entirety). Attempts to evaluate binding of compounds with this protein have repeatedly resulted in protein aggregation and precipitation, even at low micromolar protein concentrations. The difficulty in working with this protein is directly correlated with its expression protocol, and slight changes in the concentrations of Zn²⁺ in the bacterial growth media, supplemented with ZnSO₄, could lead to purified protein samples that bind with different binding affinities (K_{d}˜30 nM-2 μM) to HIF-1αC-TAD₇₈₆₋₈₂₆. To overcome this variability, ¹⁵N labeled protein was prepared and peak dispersion (and protein folding) was monitored by ¹H-¹⁵N HSQC NMR experiments (FIGS. 8A-D). This ¹⁵N-labeled, properly folded protein with the optimal levels of zinc shows a diminished tendency to aggregate and was used for binding assays.

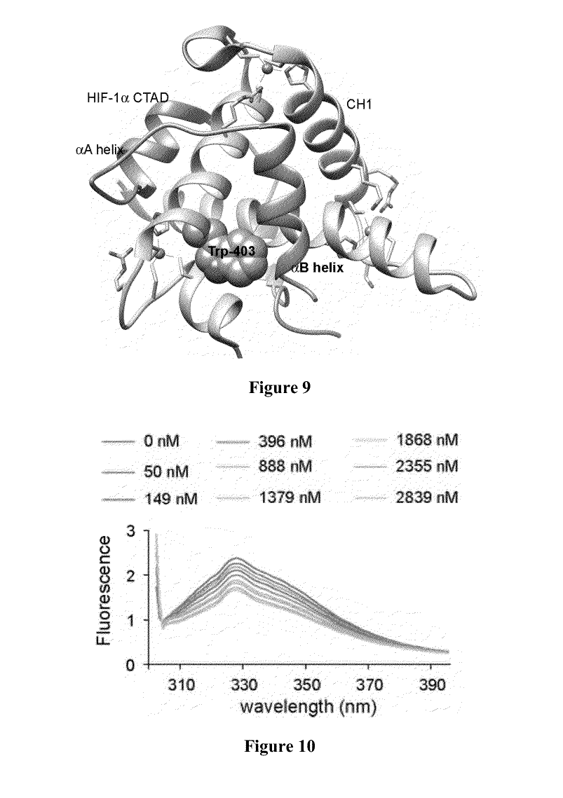

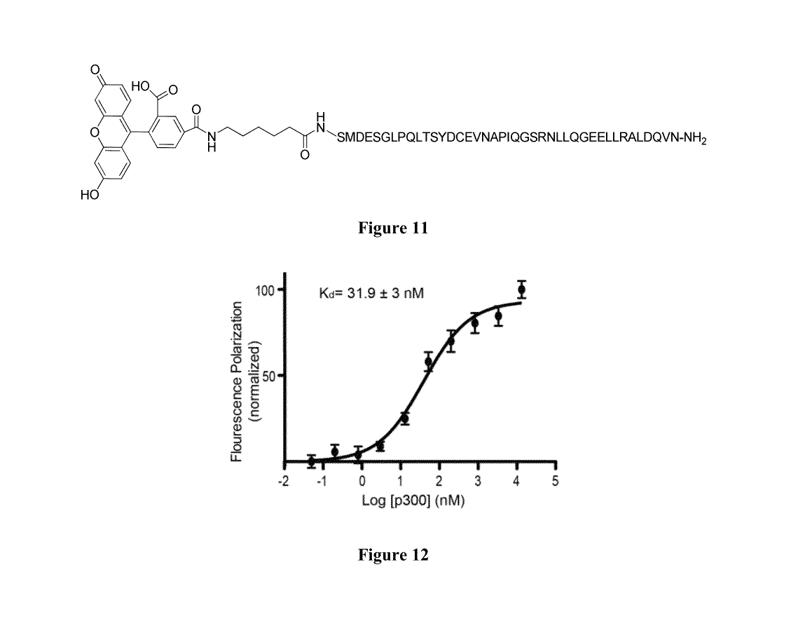

The affinity of peptides for the ¹⁵N-labeled p300 CH1 domain was evaluated using tryptophan fluorescence spectroscopy. The intrinsic fluorescence intensity of Trp403 has been shown to be a sensitive probe for CH1 folding. Significantly, this tryptophan lies in the αB binding pocket of p300/CBP, providing a unique probe for interrogating direct binding of αB mimics (FIG. 9). Using this fluorescence method, HBS 1 was calculated to bind to p300-CH1 with a dissociation constant, K_{d}, of 690±25 nM (FIG. 5A and FIG. 10). For comparison, HIF-1αC-TAD₇₈₆₋₈₂₆ binds p300-CH1 with a K_{d} of 38±0.14 nM under the same conditions. The binding affinity of HIF-1αC-TAD to CH1 in this assay is consistent with that obtained from a fluorescence polarization assay using fluorescein-labeled HIF-1αC-TAD (FIG. 11 and FIG. 12) and those using isothermal titration microcalorimetry. The designed specificity control, HBS 2, targets CH1 with a four-fold weaker binding affinity (K_{d}=2820±140 nM), supporting the computational predictions. Peptide 3 is an unconstrained analog of HBS 1 and binds the CH1 domain with a K_{d} of 6060±320 nM. These result indicate that stabilization of the peptide conformation offers a 9-fold increase in binding affinity.

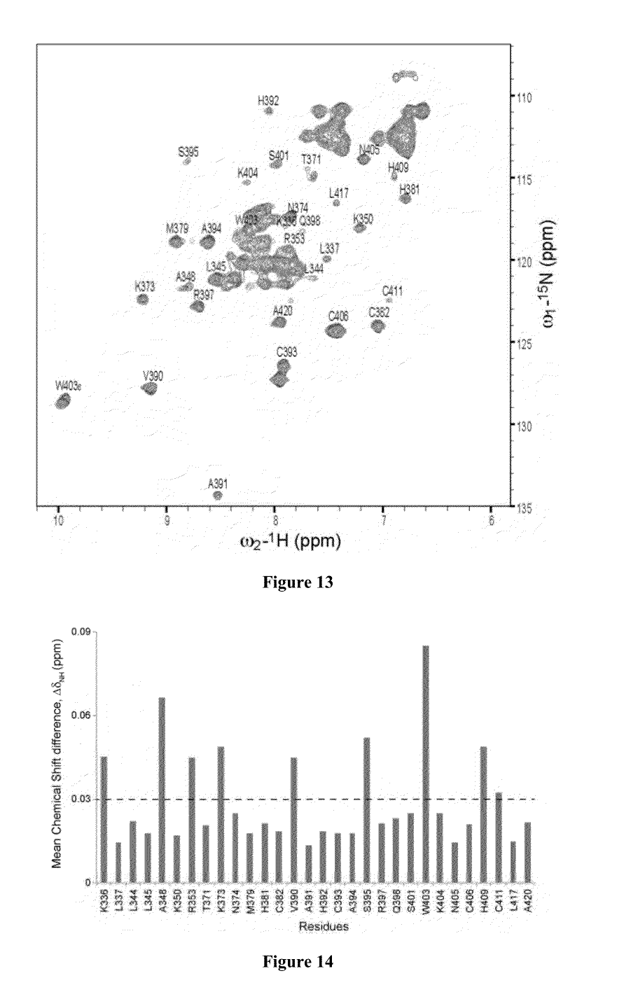

To further characterize the interaction of HBS 1 with the CH1 domain, ¹H-¹⁵N HSQC NMR titration experiments were performed with uniformly ¹⁵N-labeled CH1. Addition of HBS 1 to 69 μM CH1 in CH1:HBS 1 ratios of 1:1, 1:3, 1:5, and 1:10 resulted in a concentration-dependent shift in the resonances of several CH1 residues (FIG. 5B, FIG. 13, and FIG. 14). Specifically, addition of FIBS 1 leads to shifts in the resonances of residues corresponding to the cleft into which the αB helix of HIF binds. This cleft includes Trp403 and chemical shift perturbations observed for this residue support the results of the fluorescence titration experiments. The CH1 domain binds to numerous proteins and has been termed a scaffold for protein folding. Earlier NMR studies have suggested that Zn²⁺-bound CH1 has a relatively rigid structure, although evidence of plasticity in CH1 has also been discussed. The HSQC titration experiment with HBS 1 described herein supports the view that CH1 has a stable conformation that does not reorganize substantially, at least upon binding of small ligands. Titration of HBS 1 to zinc-bound CH1 led to a relatively large shift in the side chain indole NH of W403 as compared to the backbone amide proton of this residue, suggesting that side chain repacking governs binding of these partners.

### Example 4

### HBS 1 Disrupts the HIF-1α/p300-CH1 Complex In Vitro

A fluorescence polarization assay was used to evaluate the ability of HBS 1 to inhibit the binding of fluorescein-labeled HIF-1αC-TAD₇₈₆₋₈₂₆ domain to p300-CH1. Addition of HBS 1 to the preformed protein complex provided a concentration-dependent decrease in fluorescence polarization with an inhibitory constant, Kᵢ, of 3.5±1.2 μM (FIG. 5C). Titration of HBS 2 or peptide 3 did not lead to reproducible inhibition of the complex, as expected from their weaker affinity for the CH1 domain.

### Example 5

### HBS 1 Downregulates Hypoxia-Inducible Gene Expression and VEGF Protein Levels in Hypoxic Cells

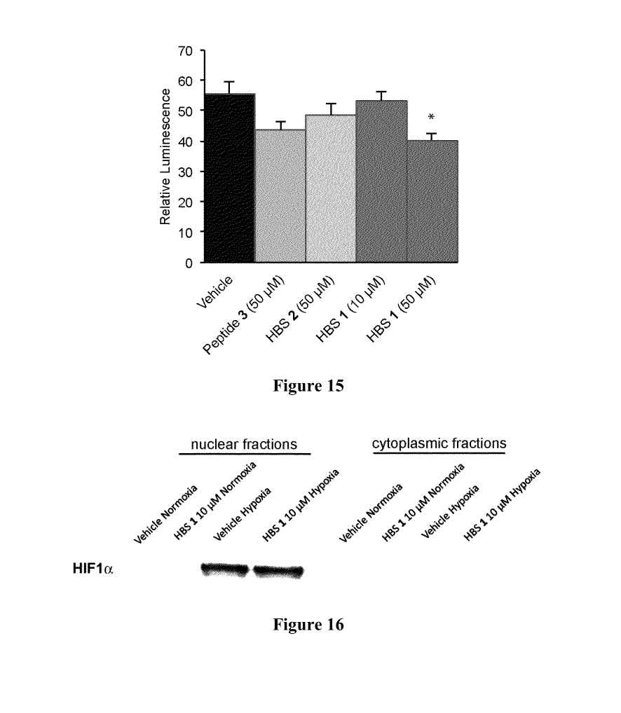

Based on the confirmed ability of HBS 1 to bind purified p300-CH1 and disrupt CH1/HIF-1αC-TAD₇₈₆₋₈₂₆ complex formation, its potential to downregulate the hypoxia-inducible promoter activity was evaluated in a luciferase-based reporter gene system. A construct containing five tandem repeats of the HRE consensus sequence found in the VEGF promoter (TACGTGGG (SEQ ID NO: 22)) cloned upstream of the hCMV minimal promoter was used to drive expression of firefly luciferase. This construct was stably transfected into a triple-negative breast cancer (TNBC) cell, MDA-MB-231, that does not express estrogen or progesterone receptors or exhibit HER-2/Neu amplification. The cells were subsequently treated with the peptides. Hypoxia was mimicked by placing cells into a GasPak EZ pouch. Under these conditions, treatment with HBS 1 at a concentration of 50 μM reduced luciferase expression by 25% (FIG. 15). At the same concentrations, specificity control HBS 2 and unconstrained peptide 3 were found to be less effective. Despite the moderate extent of inhibition of the promoter activity, these results are encouraging, because MDA-MB-231 cells are aggressive and under hypoxia conditions exhibit confluence-dependent resistance to some anticancer drugs. The luciferase reporter assays described herein suggest that treatment with HBS 1 results in a statistically significant down-regulation of HIF-1α-inducible transcription in this cell line.

To exclude the possibility that the observed down-regulation in the expression of hypoxia-inducible genes was due to a change in the levels of HIF-1α protein itself, a western blot analysis of HIF-1α was performed in hypoxic cells treated with HBS 1. HIF-1α protein was not detectable under normoxia but is strongly induced under hypoxia mimetic conditions. As expected, the levels of induced HIF-1α protein were unaffected by treatment with HBS 1 (FIG. 16).

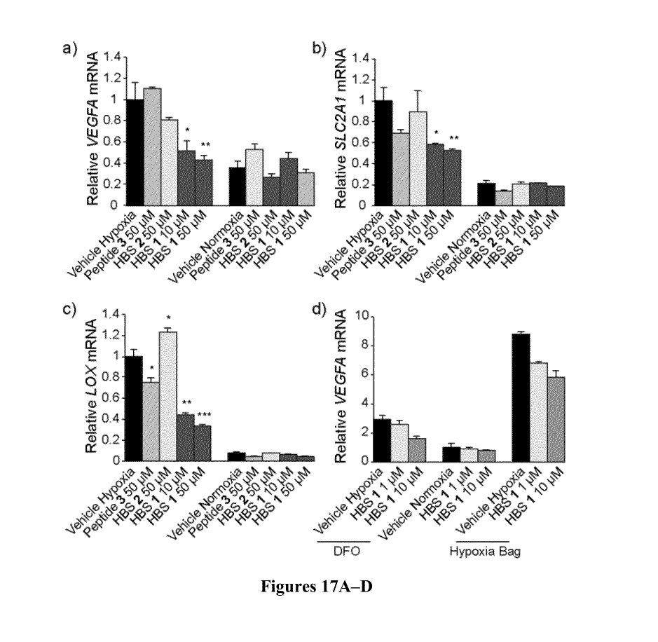

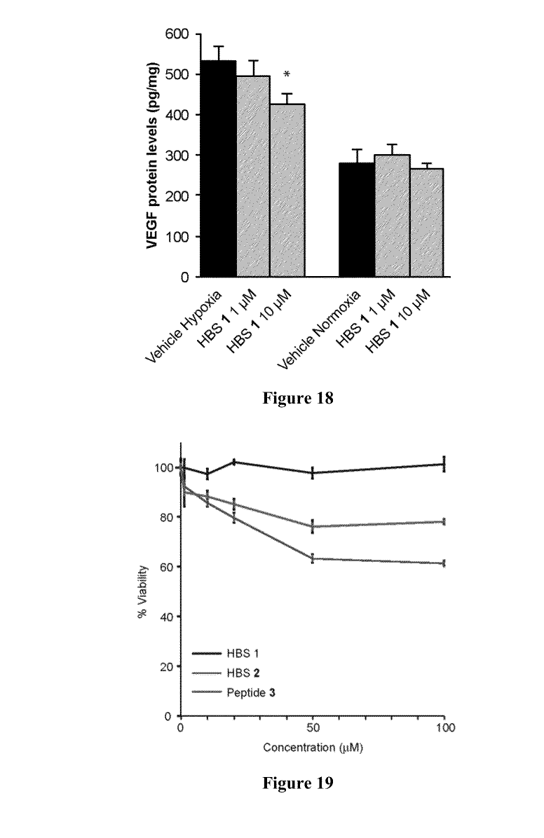

The ability of HBS 1 and HBS 2 to inhibit hypoxia-induced transcription of target genes (VEGFA, SLC2A1/GLUT-1, and LOX) was evaluated employing real-time quantitative RT-PCR (qRT-PCR) assays. The data from the qRT-PCR experiments are presented in FIGS. 17A-D. HBS 1 reduced expression levels of VEGF by 50% at 10 μM and greater than 60% at 50 μM showing marked dose dependence. In contrast, HBS 2 reduced expression levels of this gene by only 10% at 50 μM and peptide 3 was completely ineffective even at 50 μM concentration (FIG. 17A). Next, it was determined whether this inhibition could be observed for other therapeutically relevant hypoxia-inducible genes. The expression of the SLC2A1 (GLUT1) gene, one of the markers of glycolysis in tumors, and LOX, the hypoxia-inducible gene that has been shown to promote metastasis, were examined. In HeLa cells under hypoxia conditions, HBS 1 showed dose-dependent inhibition of SLC2A1 by 50-60%, comparable to that of VEGF gene in the same cell line (FIG. 17B). Similarly, HBS 1 significantly downregulated levels of expression of the LOX gene in a dose-dependent manner (55% and 70%, respectively, FIG. 17C). HBS 2 showed no activity in these assays, while peptide 3 had a reduced activity of 25%. To rule out the possibility that the compounds are only efficacious under DFO mimicked hypoxia, the efficacies of the HBS peptides in downregulating VEGF gene expression were compared under two different hypoxia mimetic conditions: DFO and prolonged incubation in an anaerobic pouch. Under both conditions, HBS 1 showed dose-dependent inhibition of VEGF expression (FIG. 17D). Next, the effect of HBS 1 treatment on the levels of secreted VEGF protein was assessed. An ELISA assay shows that HBS 1 downregulates VEGF protein levels in HeLa cells in a dose-dependent manner (FIG. 18).

HBS 1 is an efficient modulator of contacts between HIF-1α and p300/CBP. Known inhibitors of this interaction typically function allosterically, by inducing unfolding of p300/CBP through abstraction of zinc ions. This could lead to non-specific abstraction of metal ions from other biomolecules (Block et al., “Direct Inhibition of Hypoxia-Inducible Transcription Factor Complex With Designed Dimeric Epidithiodiketopiperazine,” J. Am. Chem. Soc. 131(50):18078-88 (2009), which is hereby incorporated by reference in its entirety). It was predicted that the HIF-1α mimetics should manifest their function in a more specific manner, and should not be generally cytotoxic. Cell viability assays confirm this hypothesis. It was found that HBS 1 is essentially non-cytotoxic within the entire range of tested concentrations (1 to 100 μM) (FIG. 19). Interestingly, HBS 2 shows higher level of cytotoxicity than HBS 1, suggesting that this compound may be interacting with a different set of biomolecular targets as seen from gene expression profiling data (vide infra). Thus, HBS 2 may not just be a straightforward lower affinity analog of HBS 1 as designed.

### Example 6

### Gene Expression Profiling

Proteins p300 and CBP are pleiotropic multi-domain coactivators that directly interact with multiple transcription factors. One potential limitation of the use of coactivator-targeting ligands to control gene expression is that the compounds could lead to inhibition of large numbers of genes that depend on the function of p300 or CBP. Affymetrix Human Gene ST 1.0 arrays containing oligonucleotide sequences representing over 28,000 transcripts were used to evaluate the genome-wide effects of HBS 1 and 2 under hypoxia conditions. Gene expression levels were normalized to DFO-treated cells.

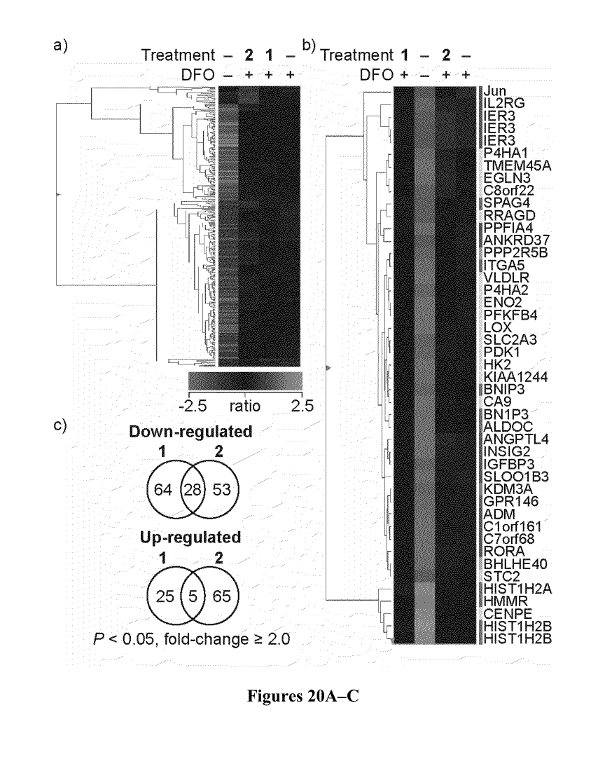

In hypoxic cells, clustering identified over 5,000 genes that changed in expression levels under one of the specified treatments: DFO, DFO+HBS 1, or DFO+HBS 2 (FIGS. 20A-C). Treatment with HBS 1 affected the expression of 122 transcripts by at least 1.1-fold (P<0.05), while at the same threshold, control HBS 2 affected expression of 155 transcripts (FIG. 20A and FIG. 20C) (see Table 5 below). Remarkably, only 33 transcripts were overlapping, indicating that the subtle difference in structure between these two compounds results in a significant difference in genome-wide effects. For comparison, DFO treatment alone affected the expression of 368 transcripts. Clustering analysis was performed to identify similarities in the expression profiles between the different treatments (FIG. 20A). The expression profile of cells treated with HBS 1 resembles the profile of cells treated with DFO under the conditions of the analysis and, as mentioned above, is different from the profile of cells treated with HBS 2 despite the structural similarity between the two compounds. As expected, the expression profile of the normoxic cells is significantly different from the other three profiles. Analysis of transcripts affected by both HBS 1 and HBS 2 shows that only 28 and 5 transcripts are commonly down- and up-regulated, respectively, by at least 1.1-fold (P<0.05). It is not surprising that there is some overlap in genes affected by both compounds given the complexity of cellular signaling pathways involved in the hypoxic response. It was found that DFO induced the expression of 45 transcripts by at least 4-fold (P<0.05) (FIG. 20B). Within this dataset, multiple genes that belong to the hypoxia-inducible pathway were identified. HBS 1 and, to some extent HBS 2, affected almost all genes in this set.

TABLE 5
Genes Affected at Least 2-Fold

| HBS 1 | | HBS 2 | | Control | | |
|---|---|---|---|---|---|---|
| Fold Change^{a} | Regulation^{b} | Fold Change^{c} | Regulation^{d} | Fold Change^{e} | Regulation^{f} | Gene |
| −1.198858 | down | 1.0873405 | up | −2.7937474 | down | ACADSB |
| −1.1193628 | down | −1.0069169 | down | −5.2134614 | down | ADM^{g} |
| −1.0377777 | down | 1.0465231 | up | −2.6769848 | down | AK4 |
| 1.0583212 | up | 1.0463357 | up | −7.2367773 | down | ALDOC |
| −1.0139477 | down | −1.0427985 | down | −2.587384 | down | ANG/RNASE4 |
| −1.2298646 | down | 1.1285037 | up | −10.575636 | down | ANGPTL4^{h} |
| −1.1789749 | down | −1.1638044 | down | −4.183105 | down | ANKRD37/UFSP2 |
| −1.0263045 | down | 1.0068687 | up | −2.3972414 | down | ANKZF1 |
| 1.031184 | up | 1.2810616 | up | −2.1560726 | down | ARHGAP28 |
| −1.0132513 | down | 1.0610821 | up | −2.1491668 | down | ARID5A |
| −1.129277 | down | 1.0992572 | up | −2.0692933 | down | ARNTL |
| −1.1177615 | down | 1.0696565 | up | −3.0109265 | down | ARRDC3 |
| −1.0990012 | down | 1.1408451 | up | 2.5837471 | up | ASF1A |
| 1.0872076 | up | −1.0366824 | down | 2.6614487 | up | ASPM |
| 1.0160675 | up | 1.1073034 | up | 2.3719292 | up | AURKA |
| −1.1789101 | down | −1.4730334 | down | −2.2839973 | down | B4GALT4 |
| −1.0872284 | down | 1.0667108 | up | −2.0636718 | down | BAMBI |
| −1.085233 | down | 1.0502583 | up | −5.271643 | down | BHLHE40 |
| 1.0080966 | up | 1.1611822 | up | −2.6880012 | down | BHLHE41 |
| 1.0014353 | up | 1.0378009 | up | −4.253393 | down | BNIP3 |
| 1.0350374 | up | 1.0081419 | up | −5.3825545 | down | BNIP3 |
| −1.0603999 | down | 1.0625899 | up | −2.9519002 | down | BNIP3L |
| 1.0250388 | up | 1.2118976 | up | 2.8752487 | up | C14orf126 |
| −1.0156919 | down | −1.119514 | down | −2.0949163 | down | C17orf76 |
| −1.122034 | down | 1.2555315 | up | −2.2554584 | down | C18orf19 |
| −1.1636652 | down | −1.0204964 | down | −6.7090216 | down | C1orf161 |
| 1.0112627 | up | 1.055041 | up | 2.4008303 | up | C1orf163 |
| 1.0871853 | up | 1.0450536 | up | −2.477235 | down | C1QL1 |
| −1.0888706 | down | 1.094631 | up | −2.9811425 | down | C3orf58 |
| 1.0302335 | up | −1.0332097 | down | −3.2238207 | down | C4orf3 |
| −1.0864575 | down | −1.0435097 | down | −2.4261425 | down | C7orf60 |
| −1.16587 | down | −1.019874 | down | −5.8869715 | down | C7orf68 |
| −1.0614659 | down | 1.2146437 | up | −4.0432177 | down | C8orf22 |
| −1.1226574 | down | −1.0902045 | down | −2.3521466 | down | CA12^{g} |
| −1.108866 | down | 1.2376684 | up | −2.48113 | down | CA5B |
| −1.0285702 | down | 1.0281031 | up | −13.296545 | down | CA9^{g} |
| −1.0158687 | down | 1.0848932 | up | −2.0076687 | down | CASZ1 |
| −1.0112811 | down | 1.0325615 | up | −2.894002 | down | CASZ1 |
| −1.04192 | down | −1.0768336 | down | −2.0998538 | down | CCDC80 |
| 1.0444043 | up | −1.1706614 | down | 3.457119 | up | CCNB1 |
| −1.0530787 | down | 1.0920707 | up | −2.177964 | down | CCNG2 |
| 1.0208882 | up | 1.2310097 | up | 2.8682868 | up | CDC20 |
| 1.0020553 | up | −1.0429283 | down | −2.8950524 | down | CDCP1 |
| 1.0366052 | up | −1.0903391 | down | −2.0103207 | down | CDK18 |
| −1.0981873 | down | 1.118337 | up | −2.5513883 | down | CDKN1A^{g} |
| 1.0392698 | up | −1.0248924 | down | 3.2755635 | up | CDKN3 |
| −1.0188439 | down | −1.0173886 | down | 2.3782046 | up | CENPA |
| −1.0183533 | down | −1.0308503 | down | 4.255259 | up | CENPE |
| −1.00388 | down | 1.118164 | up | 2.0237246 | up | CHAC2 |
| −1.0851383 | down | 1.1683263 | up | −2.276149 | down | CNOT8 |
| −1.0972031 | down | 1.0588787 | up | −2.2344368 | down | CPOX |
| 1.1451077 | up | 1.2831794 | up | −2.1453977 | down | CXCL16 |
| −1.1843526 | down | 1.1076756 | up | −2.5658453 | down | CXCR4 |
| −1.0300819 | down | 1.2587326 | up | −2.9216492 | down | DAPK1 |
| 1.0221276 | up | 1.0206687 | up | 2.4073172 | up | DDX10 |
| 1.0624138 | up | −1.0969201 | down | 2.4726677 | up | DEPDC1 |
| 1.1574726 | up | 1.0593747 | up | 2.51015 | up | DIS3L |
| −1.0776646 | down | −1.1640482 | down | −2.1677356 | down | DKFZp451A211 |
| −1.0658602 | down | 1.0331173 | up | 2.4103513 | up | DLGAP5 |
| −1.1005502 | down | −1.1672455 | down | −2.8135295 | down | DUSP5 |
| −1.3719523 | down | −1.1782583 | down | −2.2521324 | down | DUSP5P |
| 1.0034794 | up | −1.0582042 | down | −2.2900689 | down | DUSP9 |
| −1.0524148 | down | 1.2744057 | up | 2.864903 | up | E2F5 |
| −1.0750257 | down | −1.0517342 | down | −2.33422 | down | EDN2^{g} |
| −1.0114882 | down | 1.0963054 | up | −3.0769978 | down | EFNA3^{h} |
| −1.0063022 | down | 1.0588189 | up | −3.1732266 | down | EGLN1^{g} |
| −1.0551443 | down | 1.247042 | up | −5.1503882 | down | EGLN3^{g} |
| 1.0174965 | up | 1.1876371 | up | 2.072199 | up | ELOVL6 |
| −1.0733106 | down | −1.06308 | down | −10.603759 | down | ENO2^{g} |
| −1.0835003 | down | 1.0701011 | up | −2.9529157 | down | ERO1L |
| −1.1790224 | down | 1.2047563 | up | −3.7419317 | down | ERRFI1 |
| −1.1053089 | down | −1.0812296 | down | −3.2845836 | down | FAM115C |
| −1.1265318 | down | −1.0945897 | down | −3.6846316 | down | FAM115C/LOC154761 |
| −1.0174642 | down | 1.0516164 | up | 2.899864 | up | FAM133A |
| −1.1241008 | down | −1.0209429 | down | −2.5158167 | down | FAM 13A |
| −1.0018321 | down | −1.0929846 | down | −3.9271286 | down | FAM162A |
| 1.0610536 | up | −1.0276216 | down | 3.337046 | up | FAM72D/FAM72A/FAM72B/FAM72C |
| 1.0634061 | up | −1.0241611 | down | 3.3964236 | up | FAM72D/FAM72A/FAM72B/FAM72C |
| 1.0337092 | up | −1.030498 | down | 3.442782 | up | FAM72D/FAM72A/FAM72B/FAM72C |
| 1.0572628 | up | −1.0239878 | down | 3.331725 | up | FAM72D/FAM72A/FAM72B/FAM72C |
| 1.0138565 | up | −1.1081187 | down | 2.165065 | up | FAM83D |
| 1.043707 | up | −1.0308311 | down | 2.8210485 | up | FAM86B1/ALG1/LOC645332/LOC653113 |
| 1.068682 | up | −1.0393488 | down | 2.2467046 | up | FAM86B1/FAM86B2 |
| 1.0729985 | up | 1.0761985 | up | 2.617571 | up | FAM86B1/FAM86C |
| 1.0354363 | up | −1.0027288 | down | 2.0040247 | up | FAM86C |
| −1.0094354 | down | 1.3029685 | up | 2.168605 | up | FARSB |
| 1.140963 | up | −1.1433238 | down | −2.021754 | down | FBXO32 |
| −1.0524883 | down | 1.0493332 | up | −2.1420243 | down | FBXO42 |
| 1.2110548 | up | 1.0567071 | up | 2.0648973 | up | FERMT1 |
| −1.0951974 | down | −1.3137784 | down | −2.025113 | down | FN1 |
| −1.2603712 | down | −1.1467572 | down | −2.9039383 | down | FOS |
| 1.0239884 | up | −1.0724043 | down | −2.2796876 | down | FOXD1 |
| −1.0264313 | down | 1.0304923 | up | −3.194702 | down | FUT11/FLJ44715 |
| −1.038652 | down | −1.0402472 | down | −2.1154828 | down | FXYD3 |
| −1.0902228 | down | −1.1942844 | down | −2.161142 | down | FYN |
| −1.0004762 | down | 1.0612249 | up | 2.1665447 | up | G2E3 |
| −1.0668463 | down | 1.0567585 | up | −2.9950464 | down | GBE1 |
| −1.2743438 | down | 1.0132078 | up | −2.805562 | down | GDF15 |
| 1.0969177 | up | 1.0458834 | up | 2.522929 | up | GEMIN5 |
| −1.326227 | down | 1.1455405 | up | −2.8021276 | down | GFPT2 |
| −1.2790403 | down | −1.3487692 | down | −3.023291 | down | GOLGA8B/GOLGA8A |
| −1.265511 | down | −1.329899 | down | −2.905124 | down | GOLGA8B/GOLGA8A |
| 1.0313923 | up | −1.1114029 | down | 2.2596123 | up | GPATCH4 |
| −1.1066624 | down | −1.032009 | down | −5.079305 | down | GPR146 |
| −1.1756448 | down | 1.1828246 | up | −3.0940225 | down | GPR155 |
| 1.0572137 | up | −1.0137892 | down | −2.6079721 | down | GPR160 |
| −1.0540816 | down | 1.0293025 | up | −2.0090497 | down | GPRC5A |
| −1.0433288 | down | 1.0330802 | up | −2.6747115 | down | GPT2 |
| −1.089778 | down | 1.1071146 | up | −2.0776129 | down | GTF2IRD2/GTF2IRD2B |
| −1.0697725 | down | 1.1319958 | up | −2.1862717 | down | GTF2IRD2B |
| −1.0347298 | down | −1.0523677 | down | −2.2005339 | down | GYS1 |
| −1.033671 | down | 1.2477574 | up | 3.2375476 | up | H1F0 |
| −1.0744232 | down | −1.2446554 | down | −2.049202 | down | HAS2 |
| −1.1388015 | down | 1.1164491 | up | −3.110763 | down | HERC3 |
| −1.0328522 | down | −1.0706353 | down | −2.247471 | down | HEY1 |
| −1.0139298 | down | −1.1250477 | down | 2.4303255 | up | HIST1H1C |
| −1.0382714 | down | 1.2453547 | up | 2.1910377 | up | HIST1H1E |
| 1.1814293 | up | −1.0565162 | down | 4.548209 | up | HIST1H2AB |
| 1.028249 | up | 1.0202644 | up | 2.5113926 | up | HIST1H2AC |
| 1.0760688 | up | 1.0016093 | up | 2.7375612 | up | HIST1H2AE |
| 1.0772592 | up | 1.0379401 | up | 2.6594944 | up | HIST1H2AH |
| 1.088042 | up | −1.0238472 | down | 3.1786556 | up | HIST1H2AI |
| 1.0760545 | up | 1.0216632 | up | 3.3433473 | up | HIST1H2AI/HIST1H3H |
| 1.0729878 | up | 1.01679 | up | 3.328223 | up | HIST1H2AK/HIST1H2BN |
| 1.0669847 | up | 1.097534 | up | 2.094609 | up | HIST1H2AL |
| 1.1164097 | up | 1.0291708 | up | 2.655007 | up | HIST1H2BC |
| 1.0628002 | up | 1.1290944 | up | 3.553227 | up | HIST1H2BF |
| 1.0641918 | up | −1.038236 | down | 4.369798 | up | HIST1H2BG |
| 1.1094822 | up | 1.0056278 | up | 3.8479862 | up | HIST1H2BH |
| 1.0249968 | up | −1.0275244 | down | 3.34124 | up | HIST1H2BI |
| −1.011945 | down | −1.0242423 | down | 2.3438275 | up | HIST1H2BJ |
| −1.0310844 | down | 1.5378659 | up | 2.0198417 | up | HIST1H2BK |
| 1.0454845 | up | 1.0881157 | up | 2.6407917 | up | HIST1H2BK/HIST1H2BE/H2BFS |
| 1.1658144 | up | 1.0298574 | up | 12.502561 | up | HIST1H2BM |
| 1.067194 | up | 1.0149908 | up | 2.9552643 | up | HIST1H3A |
| 1.015362 | up | 1.0197564 | up | 2.8443613 | up | HIST1H3D/HIST1H2AD |
| 1.1092883 | up | −1.688728 | down | 3.1881328 | up | HIST1H3F |
| 1.0364317 | up | 1.0101742 | up | 2.6978357 | up | HIST1H3H |
| 1.1638831 | up | −1.3748453 | down | 3.6814818 | up | HIST1H4B |
| 1.0328214 | up | −1.316773 | down | 2.1798692 | up | HIST1H4H |
| 1.0930722 | up | −1.166608 | down | 3.6174786 | up | HIST1H4J/HIST1H4K |
| 1.1102779 | up | −1.1824476 | down | 3.8873343 | up | HIST1H4K/HIST1H4J |
| 1.0088866 | up | 1.0189656 | up | 2.1680818 | up | HIST2H2AA3/HIST2H2AA4/HIST2H2AC |
| 1.0085102 | up | 1.0195583 | up | 2.1686635 | up | HIST2H2AA3/HIST2H2AA4/HIST2H2AC |
| 1.2118632 | up | 1.1212372 | up | 2.66082 | up | HIST2H2AB |
| 1.0203393 | up | −1.1051214 | down | 2.6113982 | up | HIST2H2AC/BOLA1 |
| 1.0405347 | up | 1.0846759 | up | 2.0019317 | up | HIST2H2BA/HIST2H2BF |
| 1.032434 | up | 1.0164887 | up | 2.8465867 | up | HIST2H2BE |
| 1.1210366 | up | −1.0613894 | down | 2.0390704 | up | HIST2H3D/HIST2H3A/HIST2H3C |
| −1.194571 | down | 1.121211 | up | 2.1261342 | up | HIST2H4A/HIST2H4B |
| −1.1939466 | down | 1.1216862 | up | 2.1261613 | up | HIST2H4A/HIST2H4B |
| 1.0092357 | up | 1.0444485 | up | 2.094921 | up | HIST3H2A |
| −1.0752294 | down | 1.0338196 | up | −3.9728498 | down | HIVEP2 |
| −1.1303259 | down | 1.1321235 | up | −2.133175 | down | HK1^{g} |
| −1.0533404 | down | −1.0264349 | down | −5.7125254 | down | HK2^{g} |
| 1.1010088 | up | 1.1335866 | up | 4.111633 | up | HMMR |
| −1.0138937 | down | 1.2460188 | up | 2.145269 | up | HORMAD1 |
| −1.0750805 | down | −1.0380528 | down | −2.1026213 | down | HOXD10 |
| 1.1196996 | up | 1.1180418 | up | 2.7761664 | up | HPDL |
| −1.1325891 | down | 1.0055424 | up | −2.4589784 | down | HRH1 |
| −1.1192311 | down | 1.0154862 | up | 2.0284126 | up | HSPA1A/HSPA1B |
| −1.1208296 | down | 1.0085618 | up | 2.0253496 | up | HSPA1A/HSPA1B |
| −1.1177293 | down | −1.0437095 | down | 2.0822313 | up | HSPA1B/HSPA1A |
| −1.1122378 | down | −1.0498594 | down | 2.0941586 | up | HSPA1B/HSPA1A |
| −1.112368 | down | −1.0498853 | down | 2.0940685 | up | HSPA1B/HSPA1A |
| 1.2337483 | up | −1.5396951 | down | 2.4555218 | up | ID3 |
| 1.1887107 | up | −1.1141586 | down | −2.5604243 | down | IDH2 |
| −1.3824807 | down | 1.1527516 | up | −4.497776 | down | IER3 |
| −1.3749976 | down | 1.1767937 | up | −4.707593 | down | IER3 |
| −1.3823622 | down | 1.153014 | up | −4.500477 | down | IER3 |
| −1.1800597 | down | 1.0211127 | up | −4.437638 | down | IGFBP3 |
| −1.121893 | down | −1.1431843 | down | −2.0187001 | down | IGSF3 |
| −1.0634735 | down | −1.4005892 | down | −2.1043446 | down | IL1RAP |
| −1.3237284 | down | 1.0446229 | up | −5.254703 | down | IL2RG |
| −1.053483 | down | −1.0516862 | down | −2.0464828 | down | ING2 |
| −1.0689135 | down | 1.3293185 | up | −2.1875443 | down | INSIG1 |
| −1.2234808 | down | 1.0493531 | up | −7.2295227 | down | INSIG2 |
| −1.0548623 | down | 1.0914948 | up | −2.2711992 | down | IPMK |
| −1.104967 | down | −1.1261146 | down | −5.4967833 | down | ITGA5 |
| −1.2723838 | down | −1.4516369 | down | −4.7319503 | down | JUN |
| −1.0761135 | down | −1.0448471 | down | −2.08533 | down | KAT2B |
| −1.069219 | down | −1.1738038 | down | −3.964814 | down | KCTD11 |
| −1.0983505 | down | −1.0338085 | down | −4.5585265 | down | KDM3A |
| 1.0428305 | up | 1.0739981 | up | 2.0221412 | up | KIAA0586 |
| −1.0484446 | down | −1.000957 | down | −5.1322355 | down | KIAA1244 |
| −1.0863793 | down | 1.0911593 | up | −2.0486183 | down | KIAA1432 |
| −1.0647811 | down | 1.1031417 | up | −2.3516953 | down | KIAA1715 |
| −1.1583545 | down | −1.0508852 | down | 2.1151373 | up | KIF14 |
| −1.00198 | down | −1.0037445 | down | 3.4379961 | up | KIF20A/CDC23 |
| 1.1104406 | up | −1.6264613 | down | −2.1513863 | down | KRT17 |
| 1.1725253 | up | −1.8421245 | down | −2.5044134 | down | KRT17 |
| −1.0665327 | down | 1.024981 | up | −7.6152425 | down | LOX^{g} |
| −1.0239133 | down | −1.0096284 | down | −3.1381226 | down | LOXL2 |
| −1.0881166 | down | −1.1607536 | down | −2.4658139 | down | LRP1 |
| −1.0412775 | down | 1.0400462 | up | 2.0927668 | up | LTV1 |
| −1.1852337 | down | −1.4142182 | down | −2.1554024 | down | MAFB |
| −1.0526593 | down | −1.1299944 | down | −2.1507175 | down | MAFK/TMEM184A |
| 1.0472041 | up | −1.0342416 | down | 2.0437317 | up | MAK16/C8orf41 |
| −1.0581415 | down | 1.0291281 | up | −2.0293536 | down | MAP2K1/SNAPC5 |
| −1.0998803 | down | −1.0539637 | down | −2.8863204 | down | MAP3K15/PDHA1 |
| −1.1232924 | down | 1.6498638 | up | 2.2023304 | up | METTL7A |
| 1.0905137 | up | 1.0289348 | up | 2.175937 | up | MLKL |
| −1.0716023 | down | 1.0461466 | up | −2.0215554 | down | MOBKL2A |
| 1.1000898 | up | −1.0295926 | down | 2.5916712 | up | MSTO1/MSTO2P |
| 1.1853988 | up | −1.0830567 | down | 2.580263 | up | MSTO2P/MSTO1 |
| −1.0930141 | down | 1.0225625 | up | −3.293971 | down | MUC1 |
| −1.014464 | down | −1.0215149 | down | −2.346065 | down | MXI1 |
| −1.072508 | down | 1.1463394 | up | −2.0467393 | down | NAMPT |
| −1.0733447 | down | 1.1515353 | up | −2.0046158 | down | NAMPT |
| −1.0171611 | down | 1.1263885 | up | 2.024874 | up | NARS2 |
| −1.0521922 | down | −1.0211507 | down | −2.179991 | down | NAV1 |
| −1.0180564 | down | 1.0198303 | up | −3.32679 | down | NDRG1^{g} |
| 1.0553051 | up | 1.1875671 | up | 2.8213596 | up | NDUFAF4 |
| 1.0536773 | up | −1.2928615 | down | −2.193419 | down | NEBL |
| −1.0623983 | down | −1.1545544 | down | −3.262253 | down | NFIL3 |
| 1.0572549 | up | 1.0486796 | up | 2.1284277 | up | NLN |
| −1.3564715 | down | 1.412258 | up | −2.0202577 | down | NOG |
| 1.0569912 | up | −1.0584995 | down | 2.6115415 | up | NOL6 |
| 1.0345374 | up | −1.1083401 | down | 2.9392853 | up | NOP16 |
| −1.0246124 | down | −1.0321362 | down | 2.013658 | up | NOP2 |
| −1.142908 | down | −1.1094075 | down | −2.1712103 | down | NOTCH3 |
| −1.0493118 | down | 1.0916766 | up | −2.094681 | down | NRG4 |
| 1.0453973 | up | 1.0413948 | up | −2.9922984 | down | ORAI3 |
| −1.2377601 | down | −1.0565416 | down | −3.1627083 | down | OSMR |
| −1.3154866 | down | −1.2208521 | down | −2.8162463 | down | OTUD1 |
| −1.0067146 | down | 1.1961997 | up | −4.525767 | down | P4HA1 |
| −1.0518332 | down | −1.0594456 | down | −4.467532 | down | P4HA2 |
| −1.0599507 | down | −1.1152831 | down | −2.7483146 | down | PAG1 |
| −1.0068985 | down | −1.0078048 | down | −2.005217 | down | PAIP2B |
| −1.0382358 | down | 1.0521878 | up | −4.557052 | down | PDK1 |
| 1.0191542 | up | 1.1393752 | up | −3.8439698 | down | PDK3 |
| −1.085743 | down | 1.1143924 | up | −2.1830468 | down | PER1 |
| −1.0596828 | down | −1.0105202 | down | −2.0410588 | down | PER2 |
| −1.072292 | down | −1.0640502 | down | −9.719393 | down | PFKF84^{g} |
| −1.0348089 | down | 1.0235314 | up | −3.3854454 | down | PFKP |
| −1.1552294 | down | 1.008007 | up | −2.0849102 | down | PGM2L1 |
| −1.0775337 | down | 1.1154193 | up | −2.0587978 | down | PIAS2 |
| −1.0306145 | down | 1.111947 | up | 2.663589 | up | PLA2G4A |
| −1.1131518 | down | −1.1262151 | down | −3.160247 | down | PLAGL1/HYMAI |
| −1.1653019 | down | 1.0859076 | up | −2.9282918 | down | PLIN2 |
| −1.0407479 | down | 1.018729 | up | 3.1841922 | up | PLK1 |
| −1.0310947 | down | −1.0200943 | down | −2.051884 | down | PLOD1 |
| −1.0564666 | down | 1.0334756 | up | −2.6392682 | down | PLOD2 |
| −1.0536163 | down | −1.0217751 | down | −2.9487426 | down | PMEPA1 |
| −1.0364561 | down | 1.1446192 | up | 2.142106 | up | PNO1 |
| 1.0623838 | up | 1.1337379 | up | 2.3235378 | up | POLR18 |
| −1.1132654 | down | −1.2193453 | down | −5.72456 | down | PPFIA4/LOC100507405 |
| −1.0076138 | down | −1.0337875 | down | −2.3916671 | down | PPL |
| −1.0528351 | down | −1.0763819 | down | −2.0917118 | down | PPP1R3B |
| 1.0514251 | up | −1.0709876 | down | −2.185088 | down | PPP1R3C |
| −1.0886943 | down | −1.0989375 | down | −4.635631 | down | PPP2R5B |
| 1.0562048 | up | 1.0604632 | up | 2.006964 | up | PPRC1 |
| −1.1171283 | down | −1.0515006 | down | −2.7193942 | down | PRELID2 |
| 1.0199716 | up | −1.006938 | down | 2.2714365 | up | PRMT3 |
| −1.493969 | down | 1.176382 | up | −3.8572378 | down | PTGS2^{h} |
| 1.0979699 | up | −1.2585038 | down | 2.1313524 | up | PTTG1 |
| −1.084285 | down | −1.1066813 | down | −2.243677 | down | PYGL |
| 1.0216292 | up | 1.0164917 | up | −2.9913483 | down | QSOX1/FLJ23867 |
| −1.0165472 | down | 1.0808368 | up | −2.4004233 | down | RAB20 |
| −1.0168173 | down | −1.0137872 | down | −2.0478325 | down | RAB40C |
| −1.091034 | down | 1.0204805 | up | −2.3124177 | down | RAB8B |
| 1.0475962 | up | 1.1967145 | up | −3.5479445 | down | RASSF2 |
| −1.0217447 | down | 1.0673434 | up | −3.8055146 | down | RCOR2 |
| −1.0315185 | down | −1.0285182 | down | −2.5645835 | down | RIOK3 |
| −1.057626 | down | 1.006583 | up | −2.01166 | down | RIT1 |
| −1.1474804 | down | 1.0842069 | up | −2.3212476 | down | RLF |
| 1.0111393 | up | −1.0896454 | down | −3.0329592 | down | RNF122 |
| −1.0728997 | down | 1.109433 | up | −2.6993163 | down | RNF24 |
| 1.0031627 | up | −2.735376 | down | −1.2172973 | down | RNU4-2 |
| −1.1661395 | down | 1.0366671 | up | −6.9026365 | down | RORA |
| −1.0086578 | down | −1.1610154 | down | −4.0498514 | down | RRAGD |
| 1.080574 | up | 1.0721519 | up | 2.8641217 | up | RRS1 |
| 1.0885082 | up | −1.0089406 | down | 2.097932 | up | RUVBL1 |
| −1.1617795 | down | −2.3124695 | down | −1.2749236 | down | SCARNA5 |
| −1.153035 | down | −2.4092975 | down | −1.2401854 | down | SCARNA6 |
| 1.1310145 | up | 1.0209829 | up | 2.0414371 | up | SCFD2 |
| −1.0548553 | down | 1.0334789 | up | −2.9133189 | down | SEC14L4 |
| −1.0628065 | down | −1.0827417 | down | −2.5861993 | down | SEC61G |
| −1.112961 | down | −1.0562894 | down | −2.7914515 | down | SERPINE1^{g} |
| −1.163552 | down | −1.2216003 | down | −2.6976469 | down | SERPINI1 |
| −1.0688736 | down | 1.0619785 | up | −2.3279095 | down | SERTAD2 |
| 1.0343328 | up | 1.1705835 | up | 2.0703044 | up | SLC27A2 |
| −1.1072197 | down | 1.1236045 | up | −2.8005152 | down | SLC2A1^{g} |
| −1.0290065 | down | 1.053982 | up | −4.183235 | down | SLC2A3 |
| −1.248505 | down | 1.2203912 | up | −2.266289 | down | SLC6A6 |
| −1.0604588 | down | 1.0595843 | up | −2.7195609 | down | SLC6A8/SLC6A10P |
| −1.0662978 | down | 1.0684845 | up | −2.736111 | down | SLC6A8/SLC6A10P |
| −1.0853571 | down | 1.2627056 | up | 2.0000556 | up | SLC7A11 |
| −1.2457515 | down | −1.0702848 | down | −5.947674 | down | SLCO1B3/LST-3TM12 |
| −1.2625779 | down | 1.252622 | up | −2.0586894 | down | SLCO4A1 |
| −1.1349522 | down | −2.5911605 | down | −1.056101 | down | SNORA1 |
| 1.0827361 | up | −1.1856244 | down | 2.0193295 | up | SNORA13 |
| −1.1392936 | down | −2.5419006 | down | −1.22043 | down | SNORA2A |
| −1.1776297 | down | −2.339463 | down | −1.0959209 | down | SNORA42 |
| −1.0272101 | down | −2.0400264 | down | 1.1371485 | up | SNORA6 |
| −1.5449073 | down | −2.4887464 | down | −1.3515595 | down | SNORA60 |
| 1.0574348 | up | −2.9677162 | down | 1.0178032 | up | SNORA62/RPSA |
| 1.0441421 | up | −2.8423023 | down | 1.2338772 | up | SNORA74A |
| −1.2188872 | down | −3.439569 | down | 1.0985091 | up | SNORA75 |
| 1.1857955 | up | −2.4932704 | down | 1.1226008 | up | SNORD14E |
| −1.1500319 | down | −1.2012677 | down | 2.0295184 | up | SNORD1A |
| 1.1119288 | up | −2.5965233 | down | −1.0552726 | down | SNORD53 |
| 1.0002517 | up | −2.2845662 | down | −1.0211544 | down | SNORD94 |
| −1.0808043 | down | −1.0452999 | down | −2.7743483 | down | SNX33 |
| 1.0183113 | up | −1.0911404 | down | −4.066029 | down | SPAG4 |
| −1.1739895 | down | −1.00757 | down | −2.1362014 | down | SPICE1 |
| 1.0472541 | up | −1.1564113 | down | 3.817899 | up | SPINK5 |
| 1.0318233 | up | −1.0508822 | down | −2.477626 | down | SPRY1 |
| −1.1322684 | down | −1.0328805 | down | −2.0766609 | down | STAMBPL1 |
| −1.0746213 | down | 1.0388275 | up | −4.0140605 | down | STC2 |
| −1.081674 | down | 1.0557284 | up | −2.0054183 | down | SYT7 |
| 1.1072824 | up | −1.0533509 | down | 2.320572 | up | TAF9B |
| 1.1074346 | up | −1.0522659 | down | 2.320647 | up | TAF9B |
| 1.0181974 | up | 1.1829114 | up | 2.2189808 | up | TBC1D30 |
| −1.1135601 | down | 1.2469167 | up | −3.272321 | down | TCP11L2 |
| −1.0710702 | down | 1.0781746 | up | −2.2363102 | down | TET2 |
| −1.2701089 | down | −1.03623 | down | −2.419262 | down | TGFB1^{g} |
| 1.0019436 | up | −1.1744674 | down | 2.2158334 | up | TMCO7 |
| −1.0518074 | down | 1.2325256 | up | −7.3965917 | down | TMEM45A |
| −1.1878997 | down | 1.1371874 | up | −2.9050286 | down | TMEM45B |
| −1.1920087 | down | 1.2894329 | up | −2.3936403 | down | TMOD1/TSTD2 |
| 1.0234529 | up | −1.0668982 | down | −2.1390693 | down | TMPRSS3 |
| −1.0931611 | down | 1.0688102 | up | −2.3202991 | down | TNFRSF10D |
| 1.0020133 | up | −1.1098602 | down | 2.4919987 | up | TRIM59 |
| −1.0013391 | down | −1.1124156 | down | 2.0286796 | up | TROAP |
| 1.0721477 | up | 1.1957371 | up | 2.2586193 | up | TSEN2 |
| −1.0218694 | down | 1.0339891 | up | −3.1548517 | down | TTYH3 |
| −1.0063764 | down | −1.0644561 | down | 2.0020242 | up | TWISTNB |
| −1.1116943 | down | −1.1339258 | down | −2.000525 | down | UACA |
| −1.1660498 | down | −1.0305872 | down | −2.3649898 | down | UBASH3B |
| −1.0881262 | down | 1.0408965 | up | −2.4548569 | down | UPRT |
| −1.0898017 | down | 1.0274819 | up | 2.0241222 | up | UTP15 |
| −1.038064 | down | 1.0192441 | up | 2.0523014 | up | UTP20 |
| −1.0912127 | down | 1.030388 | up | −2.157118 | down | VEGFA^{g} |
| −1.0415335 | down | −1.0828103 | down | −6.05876 | down | VLDLR |
| −1.0228918 | down | 1.0196353 | up | −2.2292318 | down | VLDLR/FLJ35024 |
| −1.1679007 | down | −2.2512941 | down | 1.8234171 | up | VTRNA1-1 |
| 1.0775124 | up | −1.0424249 | down | 2.056909 | up | WDR12 |
| 1.183166 | up | 1.0591956 | up | 2.5667205 | up | WDR3 |
| −1.0831884 | down | −1.1266437 | down | 2.3755276 | up | WDR35 |
| −1.0992746 | down | 1.0805327 | up | −2.1783705 | down | WDR45L |
| −1.2116722 | down | 1.0208603 | up | −2.6231897 | down | WDR52 |
| −1.2681911 | down | −1.0793633 | down | −2.2754548 | down | WDR52 |
| −1.1009644 | down | 1.0549855 | up | −3.7621908 | down | WSB1 |
| 1.0491763 | up | 1.0669556 | up | 2.080929 | up | XK |
| −1.0340406 | down | 1.0571353 | up | −2.1956234 | down | YEATS2 |
| −1.1708703 | down | −1.0094752 | down | −2.1953986 | down | ZDBF2 |
| −1.0179691 | down | −1.0534668 | down | −2.1707454 | down | ZNF160 |
| −1.196344 | down | 1.0196155 | up | −3.2295642 | down | ZNF292 |
| 1.0262027 | up | −1.0581598 | down | −2.5864198 | down | ZNF395/FBXO16 |
| −1.1907156 | down | 1.0249686 | up | −3.4930103 | down | ZNF654/CGGBP1 |
| −1.0689389 | down | 1.0449277 | up | −2.3140717 | down | ZSWIM5 |
| 1.1991837 | up | −1.0838475 | down | 2.5001824 | up | |
| 1.1989849 | up | −1.0842861 | down | 2.501547 | up | |
| −1.0060402 | down | −3.5317602 | down | 1.2592025 | up | |
| −1.0061597 | down | −2.4166691 | down | 1.3725677 | up | |
| 1.0993127 | up | −2.1863458 | down | 1.1684631 | up | |
| −1.2011855 | down | 1.0950639 | up | −2.8855257 | down | |
| −1.1395597 | down | −2.5428736 | down | 1.2404431 | up | |
| 1.0368 | up | −2.245498 | down | −1.1451715 | down | |
| 1.0456542 | up | −2.5189538 | down | 1.2004068 | up | |
| 1.0580661 | up | −1.1550292 | down | 2.0199893 | up | |
| −1.306305 | down | −1.7012253 | down | −2.5854821 | down | |
| 1.0125278 | up | −2.0730047 | down | 1.3469207 | up | |
| −1.0938246 | down | −1.0463859 | down | −2.0193262 | down | |
| −1.0510107 | down | −1.1346707 | down | 2.074057 | up | |
| 1.1218168 | up | 1.1611335 | up | 2.0924993 | up | |
| −1.0498437 | down | −2.583383 | down | 1.0472459 | up | |

^{a}[HBS1] vs [Induced]
^{b}[HBS1] vs [Induced]
^{c}[HBS2] vs [Induced]
^{d}[HBS2] vs [Induced]
^{e}[Vehicle] vs [Induced]
^{f}[Vehicle] vs [Induced]
^{g}Hypoxia inducible
^{h}Pro-angiogenic

### Example 7

### Antitumor Activity of HBS 1 in Mouse Xenograft Models

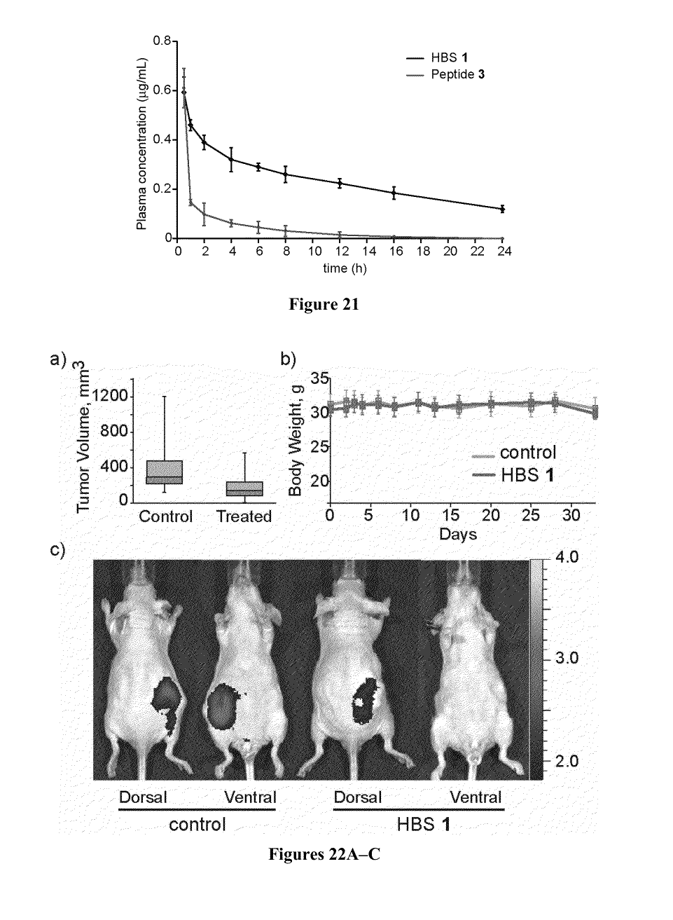

A mouse xenograft tumor model was used to assess the in vivo efficacy of HBS 1. The relative plasma stabilities of HBS 1 and linear peptide 3 in mice were first measured. In this experiment, female BALB/c mice were injected with either HBS 1 or peptide 3 at a dose of 1 mg/kg and sacrificed at various time points. Blood was collected and the plasma concentration profiles for HBS 1 and peptide 3 were determined, as shown in FIG. 21. While both compounds exhibited a bi-exponential pattern of decay, HBS 1 was retained in plasma at much higher concentrations as compared to peptide 3 during the same time intervals, suggesting that the internally constrained structure of HBS 1 favorably impacts its serum stability. This observation is consistent with the fact that proteases largely bind and cleave peptides in extended conformations. The plasma stability of HBS 1 is also consistent with the published stability of hydrocarbon-bridged helices.

The CrTac:NCr-Foxn1^{nu} mouse (Taconic, Inc.) was used for efficacy studies. 786-0 renal cell carcinoma of the clear cell type (RCC) cell line was selected, because of its high HIF levels due to a mutation in the VHL gene. Measurable tumors (˜100 mm³) grew in as little as 2-3 weeks after the inoculation of 2×10⁶ cells into the flank of the mice. Mice were then separated into the two experimental groups and one group was treated with HBS 1, whereas the second group was not treated (control). 13 mg/kg was estimated to be an acceptable dose, based on the concentration of HBS 1 required for >50% VEGF and LOX mRNA downregulation in cell culture and plasma concentrations of the compounds (vide supra). Tumor sizes were measured in accordance with literature recommendations. Throughout the course of the treatment and at the experiment endpoint, mice treated with HBS 1 had smaller tumors with median tumor volume reduction of 53% as compared to the mice from the control group (FIG. 22A). Both control group and mice treated with HBS 1 under this regimen showed no signs of distress or weight loss (FIG. 22B). To rule out the possibility that treatment resulted in a reduction of the size of the main tumor but concurrently resulted in an elevated rate of metastasis, as reported with some anti-VEGF therapeutics, the animals were injected with IR-783, a near-infrared contrast agent that targets tumors, circulating tumor cells, and metastases, and imaged from both sides using a small animal imager. The images show no detectable NIR signal within the lymph nodes, brain, or other organs and a significantly reduced signal from the main tumor (FIG. 22C).

### Discussion of Examples 1-7

Synthetic inhibitors that block the transactivation domains of transcription factors from contacting their cognate coactivators and programmable small molecules that sequence-specifically inhibit DNA-transcription factor interactions provide powerful strategies for regulating gene expression. This can be especially attractive in targeting cellular pathways that promote oncogenic transformation and typically involve a large number of signaling proteins that ultimately converge on a much smaller set of oncogenic transcription factors. Given the fact that both CBP and p300 regulate multiple signaling pathways, they provide an intriguing opportunity for an effective modulation of the expression of genes involved in cancer progression and metastasis (Vo & Goodman, “CREB-Binding Protein and p300 in Transcriptional Regulation,” J. Biol. Chem. 276(17):13505-08 (2001), which is hereby incorporated by reference in its entirety). The design strategy described herein involves judicious mimicry of transcription factor fragments that contact p300/CBP to rationally develop artificial regulators of transcription.

The present results indicate that synthetic helices that mimic protein subdomains bind their p300/CBP target with high affinity and disrupt the HIF-1αC-TAD-p300/CBP complex in vitro. Importantly, the designed compounds bound the target protein in a predictable manner; the single residue mutant HBS 2 shows an expected weaker affinity for CH1 as compared to HBS 1. The CH1 binding site for HBS 1 was confirmed by NMR HSQC titration experiments. As anticipated based on fluorescence experiments, HBS 1 causes a concentration dependent chemical perturbation shift for the side chain ε-NH of Trp-403. This result supports the design principle that a locked helix can occupy the binding clefts of individual protein α-helices. The in vitro assays showed significant reduction in promoter activity and effective downregulation of the expression of HIF-1α inducible genes responsible for promoting angiogenesis, invasion, and glycolysis. In addition, the HBS 1-mediated transcriptional blockade of VEGF correlates with decreased levels of its secreted protein product, suggesting that compensatory cellular stress response mechanisms such as internal ribosome entry sites (IRES) or mechanisms enhancing protein translation do not affect the observed downregulation in expression. Therefore, reducing the cellular mRNA levels of HIF-1α target genes with HBS 1 could be an effective means of attenuating hypoxia-inducible signaling in tumors.

Comparative analysis of the genome-wide effects of HBS 1 and HBS 2 provided additional insights into the ability of the compounds to disrupt transcriptional activity of hypoxia-inducible genes. Despite the similarity in structures, these compounds have a very different impact on the level of expression of hypoxia-inducible genes and show distinct genome-wide effects. Treatment with HBS 1 affects 122 genes (less than 0.5% of the entire transcriptome) at a fixed 1.1-fold threshold, with 92 hypoxia-inducible genes being downregulated. Despite the fact that HBS 2 has a similar genome-wide impact at the same threshold, it does not affect a majority of hypoxia-inducible genes. Because many biological responses are threshold-based, the observed decrease in transcriptional activity of primary hypoxia-inducible genes could have pronounced downstream effects on the levels of protein products of hypoxia-inducible transcription.

To assess the in vivo potential of HBS 1, murine tumor xenografts derived from the renal cell carcinoma of the clear cell type (RCC) were treated with the compound. After five injections of HBS 1, the median tumor volume was reduced by 53% in the treated group. Importantly, the HBS 1 treatment did not cause measurable changes in animal body weight or other signs of toxicity in tumor-bearing animals, nor increase the metastasis rate.

Taken together, the results reported herein support the hypothesis that designed protein domain mimetics can provide valuable tools for probing the mechanisms of transcription. Because the p300/CBP pleiotropic coactivator system interacts with diverse transcription factors, it represents an excellent model system to assess the specificity of designed synthetic ligands in gene regulation. The strategy described herein provides a foundation for the development of novel genomic tools and transcription-based therapies.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A peptidomimetic, wherein the peptidomimetic:
(i) mimics a helix having the formula X₁—X₂—X₂—X₃—X₂—X₂—X₁—X₄—X₅, wherein each X₁ is any negatively charged residue, each X₂ is any hydrophobic residue, X₃ is any positively-charged residue, X₄ is any polar residue, and X₅ is absent or any hydrophobic residue; and
(ii) is selected from the group consisting of:
(a) a compound of Formula I: 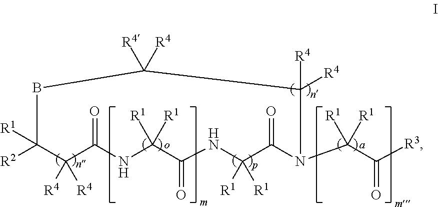
wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A: 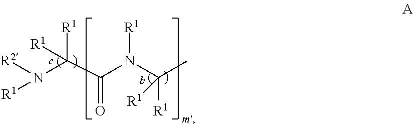
wherein:
R^{2′}is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m′ is zero or any number;
each b is independently one or two; and
c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B: 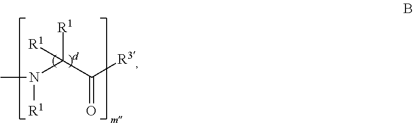
wherein:
R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m″ is zero or any number; and
each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
a is one or two;
m, n′, and n″ are each independently zero, one, two, three, or four;
m′″ is zero or one;
each o is independently one or two; and
p is one or two;
(b) a compound of Formula II: 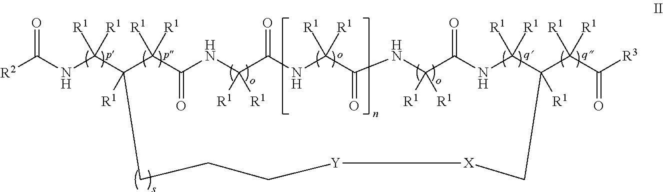
wherein:
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A: 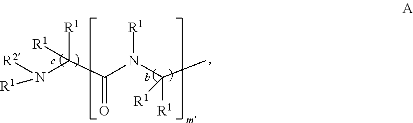
wherein:
R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m′ is zero or any number;
each b is independently one or two; and
c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B: 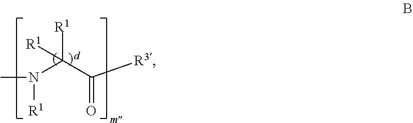
wherein:
R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m″ is zero or any number; and
each d is independently one or two;
n is one or four;
each o is independently one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond; and
(c) a compound of Formula III: 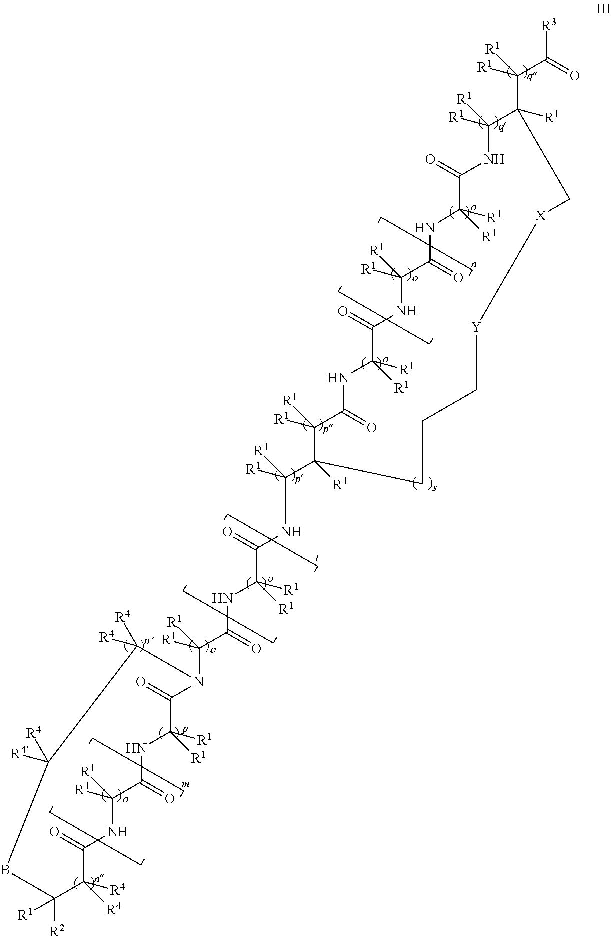
wherein:
B is C(R¹)₂, O, S, or NR¹;
each R¹ is independently hydrogen, an amino acid side chain, an alkyl, an alkenyl, an alkynyl, a cycloalkyl, a heterocyclyl, an aryl, a heteroaryl, or an arylalkyl;
R² is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula A: 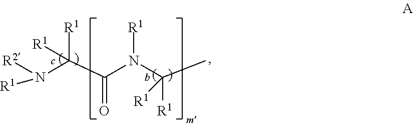
wherein:
R^{2′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —(CH₂)₀₋₁N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m′ is zero or any number;
each b is independently one or two; and
c is one or two;
R³ is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag; or a moiety of Formula B: 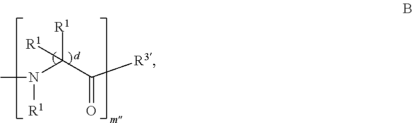
wherein:
R^{3′} is hydrogen; an alkyl; an alkenyl; an alkynyl; a cycloalkyl; a heterocyclyl; an aryl; a heteroaryl; an arylalkyl; an alpha amino acid; a beta amino acid; a peptide; a targeting moiety; a tag; —OR⁵ wherein R⁵ is hydrogen, an alkyl, an alkenyl, an alkynyl, a cycloalkyl, a heterocyclyl, an aryl, a heteroaryl, an arylalkyl, an acyl, a peptide, a targeting moiety, or a tag; or —N(R⁵)₂ wherein each R⁵ is independently hydrogen, an alkyl, an alkenyl, an alkynyl, a cycloalkyl, a heterocyclyl, an aryl, a heteroaryl, an arylalkyl, an acyl, a peptide, a targeting moiety, or a tag;
m″ is zero or any number; and
each d is independently one or two;
each R⁴ is independently hydrogen, an alkyl, an alkenyl, an alkynyl, a cycloalkyl, a heterocyclyl, an aryl, a heteroaryl, or an arylalkyl;
R^{4′} is hydrogen, an alkyl, an alkenyl, an alkynyl, a cycloalkyl, a heterocyclyl, an aryl, a heteroaryl, an arylalkyl, or a double bond between C(R^{4′}, R⁴) and B;
m, n′, and n″ are each independently zero, one, two, three, or four;
n is one or four;
each o is independently one or two;
p is one or two;
one of p′ and p″ is zero and the other is zero or one;
one of q′ and q″ is zero and the other is zero or one;
s is one, two, three, four, or five; and
Y—X is a hydrocarbon, an amide bond, an alkane, an alkene, an alkyne, a triazole, or a disulfide bond.

2. The peptidomimetic according to claim 1, wherein the peptidomimetic mimics a helix having the formula selected from the group consisting of X₁—X₂—X₂—X₃—X₂—X₂—X₁—X₄—X₅, X₁-x₂-X₂—X₃—X₂—X₂—X₁—X₄-x₅, X₁—X₂-L-X₃—X₂-L-X₁—X₄—X₅, X₁—X₂-L-X₃—X₂-L-D-X₄—X₅, X₁—X₂-L-X₃—X₂-L-X₁-Q-X₅, X₁—X₂-L-X₃—X₂-L-D-Q-X₅, and XELA*RALDQ, wherein residues in lower case bold are beta residues, X is 4-pentenoic acid, and A* is N-allylalanine.

3. The peptidomimetic according to claim 1, wherein the peptidomimetic is a compound of Formula I.

4. The peptidomimetic according to claim 3, wherein B is C(R¹)₂.

5. The peptidomimetic according to claim 3, wherein B is O.

6. The peptidomimetic according to claim 3, wherein B is S.

7. The peptidomimetic according to claim 3, wherein B is NR¹.

8. The peptidomimetic according to claim 3, wherein there are 9 to 12 atoms in the macrocycle portion of the compound.

9. The peptidomimetic according to claim 8, wherein there are 11 atoms in the macrocycle portion of the compound.

10. The peptidomimetic according to claim 3, wherein there are 12 to 15 atoms in the macrocycle portion of the compound.

11. The peptidomimetic according to claim 10, wherein there are 14 atoms in the macrocycle portion of the compound.

12. The peptidomimetic according to claim 3, wherein there are 15 to 18 atoms in the macrocycle portion of the compound.

13. The peptidomimetic according to claim 12, wherein there are 17 atoms in the macrocycle portion of the compound.

14. The peptidomimetic according to claim 3, wherein there are 20 to 24 atoms in the macrocycle portion of the compound.

15. The peptidomimetic according to claim 14, wherein there are 22 atoms in the macrocycle portion of the compound.

16. The peptidomimetic according to claim 3, wherein the peptidomimetic is a compound of Formula IA:

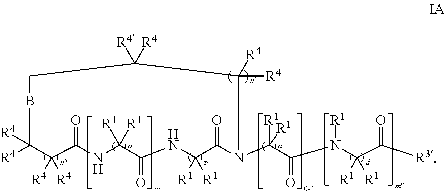

17. The peptidomimetic according to claim 3, wherein the peptidomimetic is a compound of Formula IB:

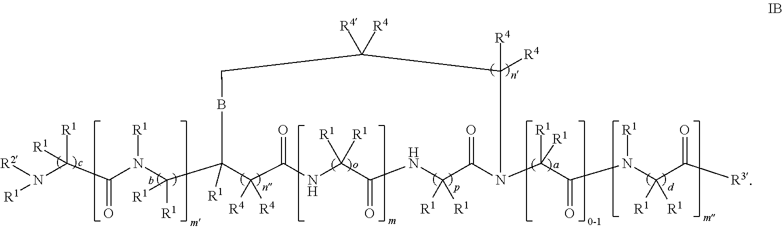

18. The peptidomimetic according to claim 3, wherein the peptidomimetic is a compound of Formula IC:

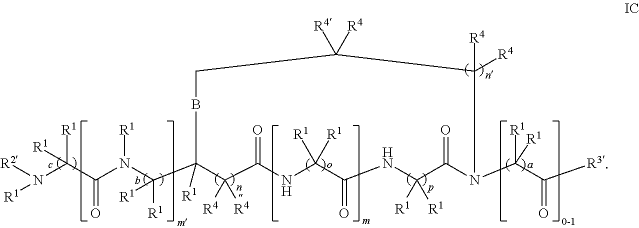

19. The peptidomimetic according to claim 1, wherein the peptidomimetic is a compound of Formula II.

20. The peptidomimetic according to claim 19, wherein the peptidomimetic is a compound of Formula IIA: 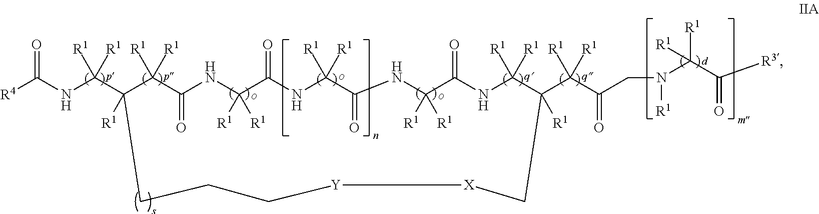
wherein R⁴ is independently hydrogen, an alkyl, an alkenyl, an alkynyl, a cycloalkyl, a heterocyclyl, an aryl, a heteroaryl, or an arylalkyl.

21. The peptidomimetic according to claim 19, wherein the peptidomimetic is a compound of Formula IIB:

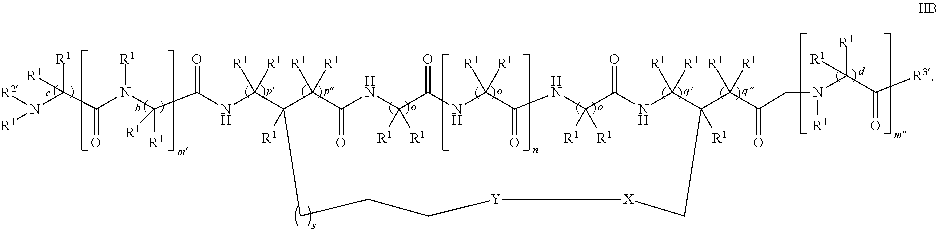

22. The peptidomimetic according to claim 19, wherein the peptidomimetic is a compound of Formula IIC:

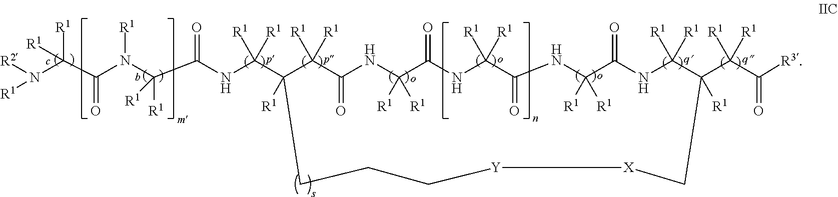

23. The peptidomimetic according to claim 1, wherein the peptidomimetic is a compound of Formula III.

24. The peptidomimetic according to claim 23, wherein the peptidomimetic is a compound of Formula IIIA:

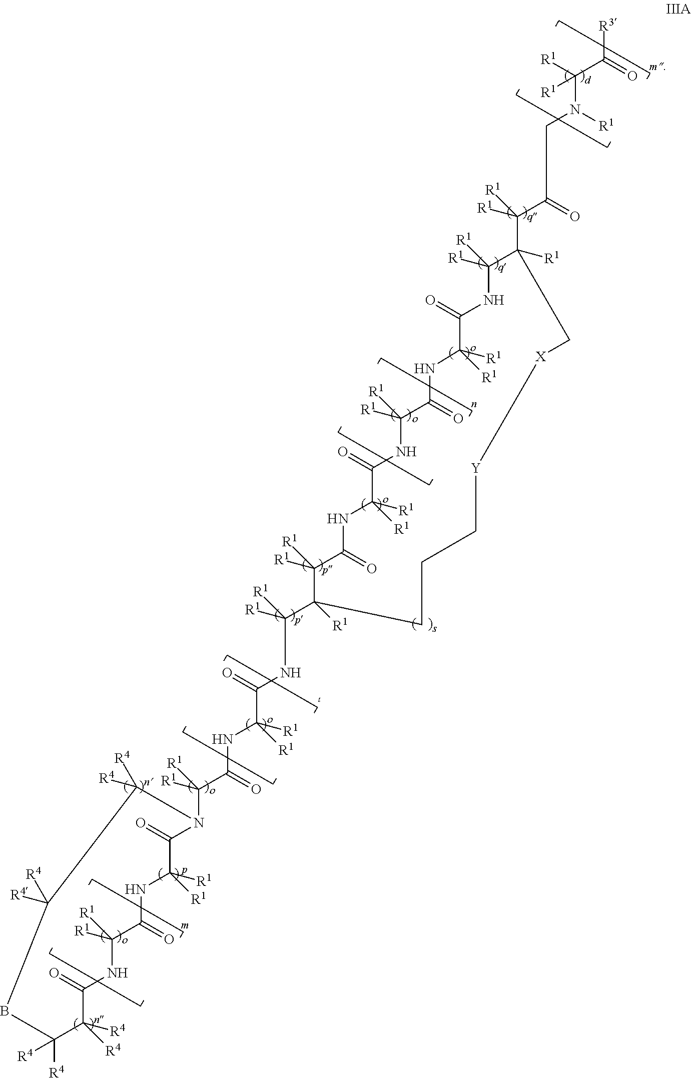

25. The peptidomimetic according to claim 23, wherein the peptidomimetic is a compound of Formula IIIB:

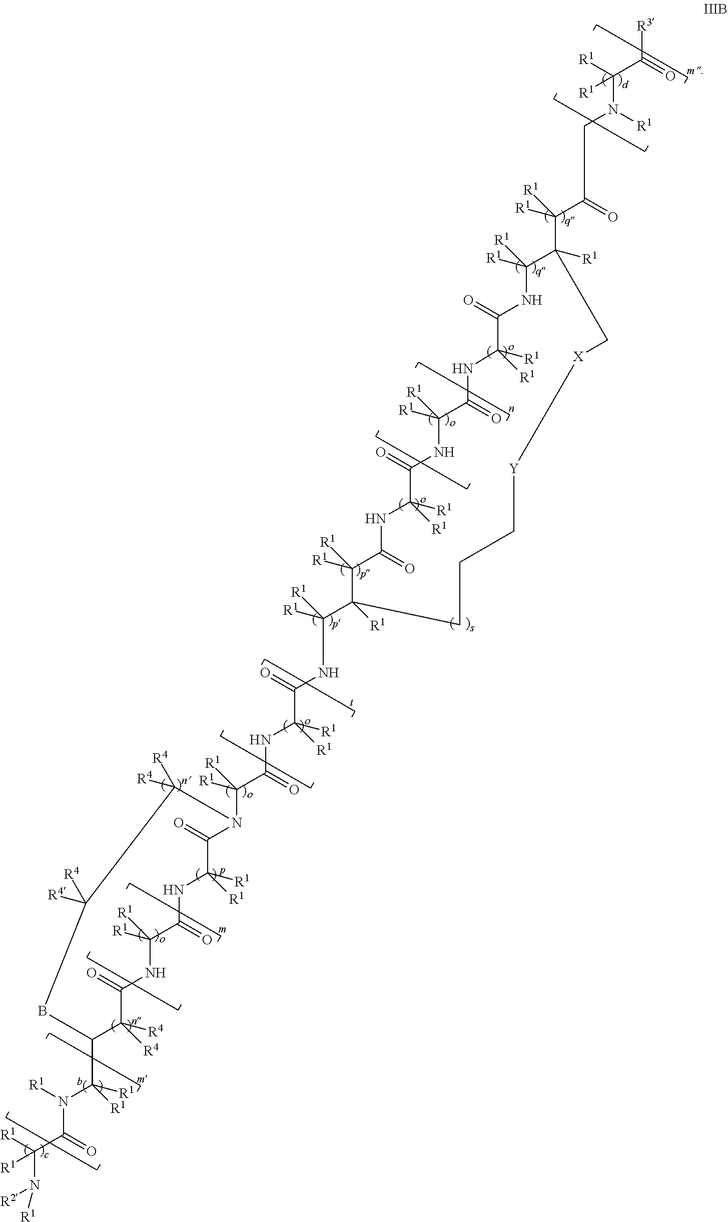

26. The peptidomimetic according to claim 23, wherein the peptidomimetic is a compound of Formula IIIC:

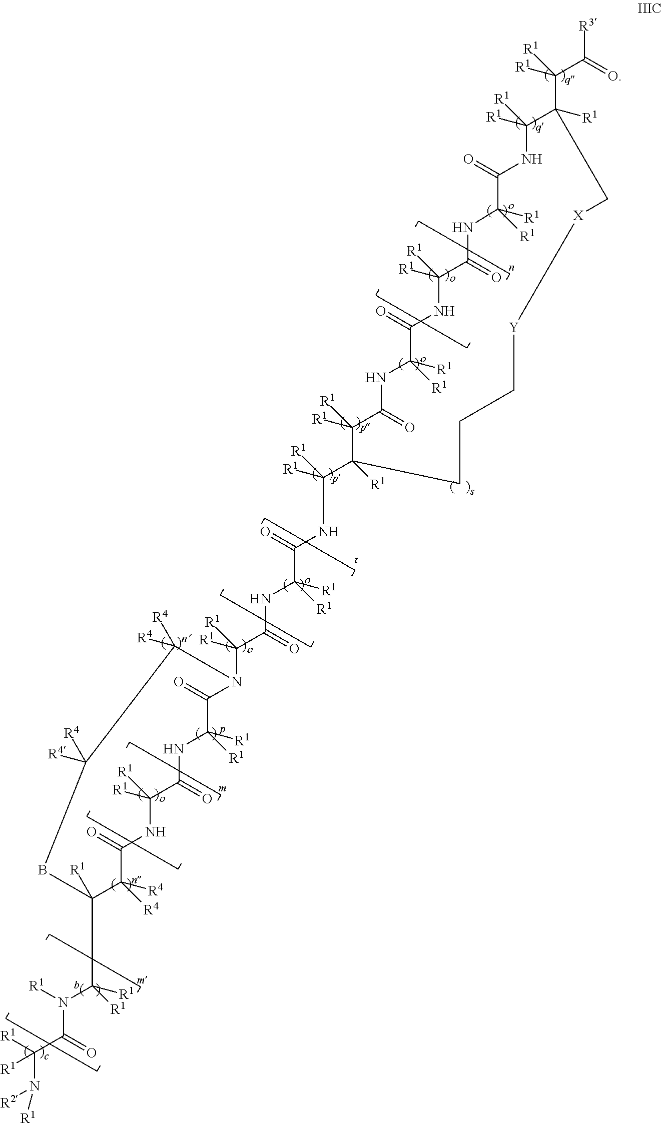

27. A pharmaceutical composition comprising a peptidomimetic according to claim 1 and a pharmaceutically acceptable vehicle.

28. The pharmaceutical composition according to claim 27, wherein the peptidomimetic is a compound of Formula I.

29. The pharmaceutical composition according to claim 28, wherein the peptidomimetic is a compound of Formula IA.

30. The pharmaceutical composition according to claim 28, wherein the peptidomimetic is a compound of Formula IB.

31. The pharmaceutical composition according to claim 28, wherein the peptidomimetic is a compound of Formula IC.

32. The pharmaceutical composition according to claim 27, wherein the peptidomimetic is a compound of Formula II.

33. The pharmaceutical composition according to claim 32, wherein the peptidomimetic is a compound of Formula IIA.

34. The pharmaceutical composition according to claim 32, wherein the peptidomimetic is a compound of Formula IIB.

35. The pharmaceutical composition according to claim 32, wherein the peptidomimetic is a compound of Formula IIC.

36. The pharmaceutical composition according to claim 27, wherein the peptidomimetic is a compound of Formula III.

37. The pharmaceutical composition according to claim 36, wherein the peptidomimetic is a compound of Formula IIIA.

38. The pharmaceutical composition according to claim 36, wherein the peptidomimetic is a compound of Formula IIIB.

39. The pharmaceutical composition according to claim 36, wherein the peptidomimetic is a compound of Formula IIIC.

40. A method of modulating transcription of a gene in a cell, wherein transcription of the gene is mediated by interaction of Hypoxia-Inducible Factor 1α with CREB-binding protein and/or p300, said method comprising:
contacting the cell with a peptidomimetic according to claim 1 under conditions effective to modulate transcription of the gene.

41. The method according to claim 40, wherein transcription is up-regulated.

42. The method according to claim 40, wherein transcription is down-regulated.

43. The method according to claim 40, wherein the gene is selected from the group of ACADSB, ADM, AK4, ALDOC, ALG1, ANG, ANGPTL4, ANKRD37, ANKZF 1, ARHGAP28, ARID5A, ARNTL, ARRDC3, ASF1A, ASPM, AURKA, B4GALT4, BAMBI, BHLHE40, BHLHE41, BNIP3, BNIP3L, BOLA1, C1orf161, C1orf163, C3orf58, C4orf3, C7orf60, C7orf68, C8orf22, C8orf41, C14orf126, C17orf76, C18orf19, C1QL1, CA12, CA5B, CA9, CASZ1, CCDC80, CCNB1, CCNG2, CDC20, CDC23, CDCP1, CDK18, CDKN1A, CDKN3, CENPA, CENPE, CGGBP1, CHAC2, CNOT8, CPOX, CXCL16, CXCR4, DAPK1, DDX10, DEPDC1, DIS3L, DKFZp451A211, DLGAP5, DUSP5, DUSP5P, DUSP9, E2F5, EDN2, EFNA3, EGLN1, EGLN3, ELOVL6, ENO2, ERO1L, ERRFI1, FAM13A, FAM72A, FAM72B, FAM72C, FAM72D, FAM83D, FAM86B1, FAM86B2, FAM86C, FAM115C, FAM115C, FAM133A, FAM162A, FARSB, FBXO16, FBXO32, FBXO42, FERMT1, FLJ23867, FLJ35024, FLJ44715, FM, FOS, FOXD1, FUT11, FXYD3, FYN, G2E3, GBE1, GDF15, GEMIN5, GFPT2, GOLGA8A, GOLGA8B, GPATCH4, GPR146, GPR155, GPR160, GPRC5A, GPT2, GTF2IRD2, GTF2IRD2B, GYS1, H1F0, H2BFS, HAS2, HERC3, HEY1, HIST1H1C, HIST1H1E, HIST1H2AB, HIST1H2AC, HIST1H2AD, HIST1H2AE, HIST1H2AH, HIST1H2AI, HIST1H2AK, HIST1H2AL, HIST1H2BC, HIST1H2BE, HIST1H2BF, HIST1H2BG, HIST1H2BH, HIST1H2BI, HIST1H2BJ, HIST1H2BK, HIST1H2BM, HIST1H2BN, HIST1H3A, HIST1H3D, HIST1H3F, HIST1H3H, HIST1H4B, HIST1H4H, HIST1H4J, HIST1H4K, HIST2H2AA3, HIST2H2AA4, HIST2H2AB, HIST2H2AC, HIST2H2BA, HIST2H2BE, HIST2H2BF, HIST2H3A, HIST2H3C, HIST2H3D, HIST2H4A, HIST2H4B, HIST3H2A, HIVEP2, HK1, HK2, HMMR, HORMAD1, HOXD10, HPDL, HRH1, HSPA1A, HSPA1B, HYMAI, ID3, IDH2, IER3, IGFBP3, IGSF3, IL1RAP, IL2RG, ING2, INSIG1, INSIG2, IPMK, ITGA5, JUN, KAT2B, KCTD11, KDM3A, KIAA0586, KIAA1244, KIAA1432, KIAA1715, KIF14, KIF20A, KRT17, LOC154761, LOC645332, LOC653113, LOC100507405, LOX, LOXL2, LRP1, LST-3TM12, LTV1, MAFB, MAFK, MAK16, MAP2K1, MAP3K15, METTL7A, MLKL, MOBKL2A, MSTO1, MSTO2P, MUC1, MXI1, NAMPT, NARS2, NAV1, NDRG1, NDUFAF4, NEBL, NFIL3, NLN, NOG, NOL6, NOP2, NOP16, NOTCH3, NRG4, ORAI3, OSMR, OTUD1, P4HA1, P4HA2, PAG1, PAIP2B, PDHA1, PDK1, PDK3, PER1, PER2, PFKFB4, PFKP, PGM2L1, PIAS2, PLA2G4A, PLAGL1, PLIN2, PLK1, PLOD1, PLOD2, PMEPA1, PNO1, POLR1B, PPFIA4, PPL, PPP1R3B, PPP1R3C, PPP2R5B, PPRC1, PRELID2, PRMT3, PTGS2, PTTG1, PYGL, QSOX1, RAB20, RAB40C, RAB8B, RASSF2, RCOR2, RIOK3, RIT1, RLF, RNASE4, RNF122, RNF24, RNU4-2, RORA, RPSA, RRAGD, RRS1, RUVBL1, SCARNA5, SCARNA6, SCFD2, SEC14L4, SEC61G, SERPINE1, SERPINI1, SERTAD2, SLC2A1, SLC2A3, SLC6A10P, SLC6A6, SLC6A8, SLC7A11, SLC27A2, SLCO1B3, SLCO4A1, SNAPC5, SNORA1, SNORA2A, SNORA6, SNORA13, SNORA42, SNORA60, SNORA62, SNORA74A, SNORA75, SNORD1A, SNORD14E, SNORD53, SNORD94, SNX33, SPAG4, SPICE1, SPINK5, SPRY1, STAMBPL1, STC2, SYT7, TAF9B, TBC1D30, TCP11L2, TET2, TGFB1, TMCO7, TMEM45A, TMEM45B, TMEM184A, TMOD1, TMPRSS3, TNFRSF10D, TRIM59, TROAP, TSEN2, TSTD2, TTYH3, TWISTNB, UACA, UBASH3B, UFSP2, UPRT, UTP15, UTP20, VEGFA, VLDLR, VTRNA1-1, WDR3, WDR12, WDR35, WDR45L, WDR52, WSB1, XK, YEATS2, ZDBF2, ZNF160, ZNF292, ZNF395, ZNF654, ZSWIM5, adenylate kinase 3, α_{1B}-adrenergic receptor, aldolase A, ceruloplasmin, c-Met protooncogene, CXCL12/SDF-1, endothelin-1, enolase 1, erythropoietin, glucose transporter 1, glucose transporter 3, glyceraldehyde-3-phosphate dehydrogenase, heme oxygenase 1, IGF binding protein 1, insulin-like growth factor 2, lactate dehydrogenase A, nitric oxide synthase 2, p35^{srg}, phosphoglycerate kinase 1, pyruvate kinase M, transferrin, tranferrin receptor, transforming growth factor β₃, vascular endothelial growth factor, vascular endothelial growth factor receptor FLT-1, and vascular endothelial growth factor receptor KDR/Flk-1.

44. A method of treating or preventing in a subject a disorder mediated by interaction of Hypoxia-Inducible Factor 1α with CREB-binding protein and/or p300, said method comprising:
administering to the subject a peptidomimetic according to claim 1 under conditions effective to treat or prevent the disorder.

45. The method according to claim 44, wherein the disorder is selected from the group of abnormal vasoconstriction, retinal ischemia, pulmonary hypertension, intrauterine growth retardation, diabetic retinopathy, age-related macular degeneration, diabetic macular edema, and cancer.

46. A method of reducing or preventing angiogenesis in a tissue, said method comprising:
contacting the tissue with a peptidomimetic according to claim 1 under conditions effective to reduce or prevent angiogenesis in the tissue.

47. The method according to claim 46, wherein the method is carried out in vivo.

48. The method according to claim 46, wherein the tissue is a tumor.

49. A method of decreasing survival and/or proliferation of a cell under hypoxic conditions, said method comprising:
contacting the cell with a peptidomimetic according to claim 1 under conditions effective to decrease survival and/or proliferation of the cell.

50. The method according to claim 49, wherein the cell is cancerous or is contained in the endothelial vasculature of a tissue that contains cancerous cells.

51. A method of identifying a potential ligand of CREB-binding protein and/or p300, said method comprising:
providing a peptidomimetic according to claim 1,
contacting the peptidomimetic with a test agent, and
detecting whether the test agent selectively binds to the peptidomimetic, wherein a test agent that selectively binds to the peptidomimetic is identified as a potential ligand of CREB-binding protein and/or p300.


SEQUENCE LISTING (11 sequences)

SEQ ID NO: 1 - protein, 41 aa, Homo sapiens
SMDESGLPQLTSYDCEVNAPIQGSRNLLQGEELLRALDQVN

SEQ ID NO: 2 - protein, 34 aa, Homo sapiens
PQLTSYDCEVNAPIQGSRNLLQGEELLRALDQVN

SEQ ID NO: 3 - protein, 8 aa, Artificial
  Unconstrained peptide
ELARALDQ

SEQ ID NO: 4 - protein, 29 aa, Artificial
  Signal peptide
MMSFVSLLLVGILFYATEAEQLTKCEVFQ

SEQ ID NO: 5 - protein, 4 aa, Artificial
  Endoplasmic reticulum retention signal
KEDL

SEQ ID NO: 6 - protein, 8 aa, Artificial
  Nuclear transport peptide
PPKKKRKV

SEQ ID NO: 7 - protein, 25 aa, Artificial
  Mitochondrial transport peptide
MLSLRQSIRFFKPATRTLCSSRYLL

SEQ ID NO: 8 - protein, 9 aa, Artificial
  Helix
XXLXXLDQX

SEQ ID NO: 9 - protein, 8 aa, Artificial
  Unconstrained peptide
ELARALDQ

SEQ ID NO: 10 - protein, 8 aa, Homo sapiens
ELLRALDQ

SEQ ID NO: 11 - DNA, 8 bp, Artificial
  VEGF promoter HRE consensus sequence
tacgtggg